SEQUENCE LISTING (55 sequences)

SEQ ID NO: 1 - DNA, 1233 bp, Artificial
  recombinant fusion protein construct
atggccgacgaggcaccacaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacactagtacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcgaggactag

SEQ ID NO: 2 - protein, 410 aa, Artificial
  recombinant fusion protein
MADEAPQGSRSLTPCTCGSSDLYLVTRHADVIPVRRRGDSRGSLLSPRPISYLKGSSGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYTSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVED

SEQ ID NO: 3 - DNA, 1914 bp, Artificial
  recombinant fusion protein construct
atggccgacgaggcaccagcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctccgctgggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgtag

SEQ ID NO: 4 - protein, 637 aa, Artificial
  recombinant fusion protein
MADEAPAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSAGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVT

SEQ ID NO: 5 - DNA, 1491 bp, Artificial
  recombinant fusion protein construct
atggccgacgaggcaccataccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatggaaacccacgtcaccgggggaagtgccggccgcaccacggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtag

SEQ ID NO: 6 - protein, 496 aa, Artificial
  recombinant fusion protein
MADEAPYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWE

SEQ ID NO: 7 - DNA, 483 bp, Artificial
  recombinant fusion protein construct
atggccgacgaggcaccatctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttctgggcgaagcacatgtggaatttcatcagtgggatacaatacttggcgggcctgtcaactagtcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctag

SEQ ID NO: 8 - protein, 160 aa, Artificial
  recombinant fusion protein
MADEAPSQHLPYIEQGMMLAEQFKQKALGLLQTASRHAEVITPAVQTNWQKLEVFWAKHMWNFISGIQYLAGLSTSPGALVVGVVCAAILRRHVGPGEGAVQWMNRLIAFASRGNHVSPTHYVPESDAAARVTAILSSLTVTQLLRRLHQWISSECTTPC

SEQ ID NO: 9 - protein, 6 aa, Artificial
  synthetic peptide
MADEAP

SEQ ID NO: 10 - protein, 9 aa, Artificial
  synthetic peptide
GGGHHHHHH

SEQ ID NO: 11 - DNA, 2256 bp, Artificial
  recombinant fusion protein construct
atggccgacgaggcaccaagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaagtgccggccgcaccacggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttcctcctgcttgcagacgcgcgcgtctgctcctgcttgtggatgatgttactcatatcccaagcggaggcgtag

SEQ ID NO: 12 - protein, 751 aa, Artificial
  recombinant fusion protein
MADEAPSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 13 - DNA, 1908 bp, Artificial
  recombinant fusion protein construct
atggccgacgaggcaccaagtacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatggaaacccacgtcaccgggggaagtgccggccgcaccacggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtag

SEQ ID NO: 14 - protein, 635 aa, Artificial
  recombinant fusion protein
MADEAPSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWE

SEQ ID NO: 15 - DNA, 2538 bp, Artificial
  recombinant fusion protein construct
atggccgacgaggcaccagcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctccgctgggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggattgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttctgggcgaagcacatgtggaatttcatcagtgggatacaatacttggcgggcctgtcaactagtcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctag

SEQ ID NO: 16 - protein, 845 aa, Artificial
  recombinant fusion protein
MADEAPAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSAGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRHAEVITPAVQTNWQKLEVFWAKHMWNFISGIQYLAGLSTSPGALVVGVVCAAILRRHVGPGEGAVQWMNRLIAFASRGNHVSPTHYVPESDAAARVTAILSSLTVTQLLRRLHQWISSECTTPC

SEQ ID NO: 17 - DNA, 2982 bp, Artificial
  recombinant fusion protein construct
atggccgacgaggcaccatccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacataggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggccagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacccgcagaaattctgcggaagtctcggagattcgcccgggccctgcccgtttgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatatag

SEQ ID NO: 18 - protein, 993 aa, Artificial
  recombinant fusion protein
MADEAPSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPI

SEQ ID NO: 19 - DNA, 9599 bp, Hepatitis C virus
gccagccccctgatgggggcgacactccaccatgaatcactcccctgtgaggaactattgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaaatgccggccgcaccacggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttgaccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggacgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggcctacgccttctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgttttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgcaggcgcccatcacggcgtacacccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattggcaccctgcacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaggcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccattcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggaccggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttctatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggcgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacttgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgaacctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcaccccaataacggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgt

SEQ ID NO: 20 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGNAGRTTAGLVGLLTPGAKQNIQLINTNGSWHINSTALNCNESLNTGWLAGLFYQHKFNSSGCPERLTSCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAAYAFYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLQAPITAYTQQTRGLLGCIITSLTGRDKNQVEGEVQIVSTATQTFLATCINGVCWTVYHGAGTRTIASPKGPVIQMYTNVDQDLVGWPAPQGSRSLAPCTCGSSDLYLVTRHADVIPVRRRGDSRGSLLSPRPISYLKGSSGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRHAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAANLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPITAAGRLDLSGWFTAGYSGGDIYHSVSHARPRWFWFCLLLLAAGVGIYLLPNR

SEQ ID NO: 21 - DNA, 20 bp, Artificial
  primer
tatgtcagcgcccacaattc

SEQ ID NO: 22 - DNA, 20 bp, Artificial
  primer
ggctcagggtcaatcacaga

SEQ ID NO: 23 - DNA, 20 bp, Artificial
  primer
ggaaggagcagttgcgctgc

SEQ ID NO: 24 - protein, 212 aa, Hepatitis B virus
MQLFHLCLIISCTCPTFQASKLCLGWLWGMDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRCRGRSPRRRTPSPRRRRSQSPRRRRSKSRESQC

SEQ ID NO: 25 - protein, 845 aa, Hepatitis B virus
MPLSYQHFRKLLLLDDGTEAGPLEEELPRLADADLNRRVAEDLNLGNLNVSIPWTHKVGNFTGLYSSTVPIFNPEWQTPSFPKIHLQEDIINRCQQFVGPLTVNEKRRLKLIMPARFYPTHTKYLPLDKGIKPYYPDQVVNHYFQTRHYLHTLWKAGILYKRETTRSASFCGSPYSWEQELQHGRLVIKTSQRHGDESFCSQPSGILSRSSVGPCIRSQLKQSRLGLQPHQGPLASSQPGRSGSIWARAHPSTRRYFGVEPSGSGHIDHSVNNSSSCLHQSAVRKAAYSHLSTSKRQSSSGHAVEFHCLPPSSAGSQSQGSVFSCWWLQFRNSKPCSEYCLSHLVNLREDWGPCDEHGEHHIRIPRTPARVTGGVFLVDKNPHNTAESRLVVDFSQFSRGSTRVSWPKFAVPNLQSLTNLLSSNLSWLSLDVSAAFYHIPLHPAAMPHLLIGSSGLSRYVARLSSNSRINNNQYGTMQNLHDSCSRQLYVSLMLLYKTYGWKLHLYSHPIVLGFRKIPMGVGLSPFLLAQFTSAICSVVRRAFPHCLAFSYMDDVVLGAKSVQHRESLYTAVTNFLLSLGIHLNPNKTKRWGYSLNFMGYIIGSWGTLPQDHIVQKIKHCFRKLPVNRPIDWKVCQRIVGLLGFAAPFTQCGYPALMPLYACIQAKQAFTFSPTYKAFLSKQYMNLYPVARQRPGLCQVFADATPTGWGLAIGHQRMRGTFVAPLPIHTAELLAACFARSRSGAKLIGTDNSVVLSRKYTSFPWLLGCTANWILRGTSFVYVPSALNPADDPSRGRLGLSRPLLRLPFQPTTGRTSLYAVSPSVPSHLPVRVHFASPLHVAWRPP

SEQ ID NO: 26 - protein, 400 aa, Hepatitis B virus
MGGWSSKPRKGMGTNLSVPNPLGFFPDHQLDPAFGANSNNPDWDFNPIKDHWPTANQVGVGAFGPGLTPPHGGILGGSPQAQGILTTVSTIPPPASTNRQSGRQPTPISPPLRDSHPQAMQWNSTAFHQALQDPRVRGLYFPAGGSSSGTVNPAPNIASHISSISARTGDPVANMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 27 - protein, 154 aa, Hepatitis B virus
MAARLYCQLDSSRDVLCLRPVGAESRGRPLSGPLGTLSSPSPSAVPADHGAHLSLRGLPVCAFSSAGPCALRFTSARCMETTVNAHQILPKVLHKRTLGLPAMSTTDLEAYFKDCVFKDWEELGEEIRLKVFVLGGCRHKLVCAPAPCNFFTSA

SEQ ID NO: 28 - protein, 212 aa, Hepatitis B virus
MQLFHLCLIISCSCPTVQASKLCLGWLWGMDIDPYKEFGATVELLSFLPSDFFPSVRDLLDTAAALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 29 - protein, 843 aa, Hepatitis B virus
MPLSYQHFRKLLLLDDEAGPLEEELPRLADEGLNHRVAEDLNLGNPNVSIPWTHKVGNFTGLYSSTVPVFNPEWQTPSFPDIHLQEDIVDRCKQFVGPLTVNENRRLKLIMPARFFPNVTKYLPLDKGIKPYYPEHVVNHYFQTRHYLHTLWKAGILYKRESTHSASFCGSPYSWEQDLQHGRLVFQTSKRHGDKSFCPQSPGILPRSSVGPCIQSQLRKSRLGPQPTQGQLAGRPQGGSGSIRARVHPSPWGTVGVEPSGSGHTHICASSSSSCLHQSAVRKAAYSLISTSKGHSSSGHAVELHHFPPNSSRSQSQGSVLSCWWLQFRNSKPCSEYCLYHIVNLIEDWGPCAEHGEHRIRTPRTPARVTGGVFLVDKNPHNTAASRLVVDFSQFSRGNTRVSWPKFAVPNLQSLTNLLSSNLSWLSLDVSAAFYHLPLHPAAMPHLLVGSSGLSRYVARLSSNSRIINHQHGTMQDLHNSCSRNLYVSLMLLYKTYGWKLHLYSHPIILGFRKIPMGVGLSPFLLAQFTSAICSVVRRAFPHCLAFSYMDDVVLGAKSVQHLESLYAAVTNFLLSLGIHLNPHKTKRWGYSLNFMGYVIGSWGTLPQEHIVQKIKLCFRKIPVNRPIDWKVCQRIVGLLGFAAPFTQCGYPALMPLYACIQAKQAFSFSPTYKAFLSKQYLTLYPVARQRPGLCQVFADATPTGWGLAIGHQRMRGTFVSPLPIHTAELLAACFARSRSGAKLIGTDNSVVLSRKYTSFPWLLGCAANWILRGTSFVYVPSALNPADDPSRGRLGLYRPLLRLPYRPTTGRTSLYPDSPSVPSHLPDRVHFASPLHVAWKPP

SEQ ID NO: 30 - protein, 400 aa, Hepatitis B virus
MGGWSSKPRKGMGTNLSVPNPLGFFPDHQLDPAFKANSDNPDWDLNPHKDNWPDSNKVGVGAFGPGFTPPHGGLLGWSPQAQGMITTVPAAPPPASTNRQLGRQPTPLSPPLRDSHPQAMQWNSTTFHKTLQDPRVRALYFPAGGSSSGTVNPVQNTASSISSILSKTGDPVPNMENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCRTCTTPAQGTSMFPSCCCTKPTDGNCTCIPIPSSWAFAKYLWEWASVRFSWLSLLAPFVQWFVGLSPTVWLSVIWMMWFWGPSLYNILSPFMPLLPLFFCLWVYI

SEQ ID NO: 31 - protein, 155 aa, Hepatitis B virus
MAARLCCQLDPARDVLCLRPVGAESRGRPLPGPLGALPPASPSAVPTDHGAHLSLPGLPVCAFSSAGPCALRFTSARRMETTVNAHRNLPKVLHKRTLGLSAMSTTDLEAYFKDCVFNEWEELGEEIRLKVLFVLGGCRHKLVCSPAPCNFFTSA

SEQ ID NO: 32 - protein, 212 aa, Hepatitis B virus
MQLFPLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 33 - protein, 843 aa, Hepatitis B virus
MPLSYQHFRKLLLLDDEAGPLEEELPRLADEGLNRRVAEDLNLGNLNVSIPWTHKVGNFTGLYSSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRSVFQTSKRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSTRSQSEGPIFSCWWLQFRNSKPCSDYCLTHIVNLLEDWGPCTEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVQKLKQCFRKLPVNRPIDWKVCQRIVGLLGFAAPFTQCGYPALMPLYACIQAKQAFTFSPTYKAFLCKQYLNLYPVARQRSGLCQVFADATPTGWGLAIGHQRMRGTFVAPLPIHTAELLAACFARSRSGAKLIGTDNSVVLSRKYTSFPWLLGCTANWILRGTSFVYVPSALNPADDPSRGRLGLYRPLLRLPFRPTTGRTSLYADSPSVPSHLPDRVHFASPLHVAWKPP

SEQ ID NO: 34 - protein, 400 aa, Hepatitis B virus
MGGWSSKPRKGMGTNLSVPNPLGFFPDHQLDPAFGANSNNPDWDFNPNKDHWPEANQVGVGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILNPFLPLLPIFFCLWVYI

SEQ ID NO: 35 - protein, 154 aa, Hepatitis B virus
MAARVCCKLDPARDVLCLRPVGAESSGRPVSGPFGTLPSPSSSAVPADHGAHLSLRGLPVCAFSSAGPCALRFTSARRMETTVNAHQVLPKVLHKRTLGLSAMSTTDLEAYFKDCVFKDWEELGEEIRLKVFVLGGCRHKLVCSPAPCNFFTSA

SEQ ID NO: 36 - protein, 212 aa, Hepatitis B virus
MQLFHLCLIISCSCPTVQASKLCLGWLWDMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 37 - protein, 832 aa, Hepatitis B virus
MPLSYQHFRRLLLLDPDAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLEKGIKPYYPEHLVNHYFQTRHYLHTLWKAGILYKRETTRSASFCGSPYSWEQELQHGAESFHQQSSGILSRPPVGSSLQSKHRKSRLGLQSQQGHLARRQQGRSWSIRAGIHPTARRPFGVEPSGSGHTANLANKSASCLYQSSVRKAAYSSVSTFEKHSSSSNAVELHNLPPNPARSQSERPVFPCWWLQFRNSKPCSDYCLSHIVNLLEDWGPCAEHGEHHIRIPRTPARVTGGVFLVDKNPHNTVESRLVVDFSQFSRGNHRVSWPKFAVPNLQSLTNLLSSNLSWLSLDVSAAFYHLPLHPAAMPHLLVGSSGLSRYVARLSSNSRIFNHQHGTMQNLHDSCSRNLYVSLLLLYQTFGRKLHLYSHPIILGFRKIPMGVGLSPFLLAQFTSAICSVVRRAFPHCLAFSYMDDVVLGAKSVQHLESLFTAVTNFLLSLGIHLNPNKTKRWGYSLHFMGYVIGSYGSLPQDHIRQKIKECFRKLPVNRPIDWKVCQRIVGLLGFAAPFTQCGYPALKPLYACIQSKQAFTFSPTYKAFLCKQYLNLYPVARQRPGLCQVFADATPTGWGLVMGHQRMRGTFLDPLPIHTAELLAACFARSRSGANILGTDNSVVLSRKYTSFPWLLGCAANWILRGTSFVYVPSALNPADDPSRGRLGLSRPLLRLPFRPTTGRTSLYADSPSVPSHLPDRVHFASPLHVAWRPP

SEQ ID NO: 38 - protein, 389 aa, Hepatitis B virus
MGQNLSTSNPLGFFPDHQLDPAFRANTANPDWDFNPNKDTWPDANKVGAGAFGLGFTPPHGGLLGWSPQAQGILQTLPANPPPASTNRQSGRQPTPLSPPLRNTHPQAMQWNSTTFHQTLQDPRVRGLYFPAGGSSSGTVNPVPTTVSHTSSIFSRIGDPALNMENITSGLLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNILSPFLPLLPIFFCLWVYI

SEQ ID NO: 39 - protein, 154 aa, Hepatitis B virus
MAARLCCQLDPARDVLCLRPVSAESCGRPVSGPFGTLSSPSPSAVSTDHGAHLSLRGLPVCAFSSAGPCALRFTSARRMETTVNAHQFLPKVLHKRTLGLSVMSTTDLEAYFKDCLFKDWEELGEETRLMIFVLGGCRHKLVCAPAPCNFFTSA

SEQ ID NO: 40 - protein, 212 aa, Hepatitis B virus
MQLFHLCLIISCSCPTVQASKLCLGWLWGMDIDPYKEFGATVELLSFLPSDFFPSVRDLLDTASALYRDALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPENTVVRRRGRSPRRRTPSPCRRRSQSPRRRRSQSPASQC

SEQ ID NO: 41 - protein, 842 aa, Hepatitis B virus
MPLSYQHFRRILLLDEEAGPLEEELPRLADEDLNRRVAEDLNLQLPNVSIPWTHKVGNFTGLYSSTVPVFNPNWKTPSFPDIHLHQDIINKCEQLVGPLTVNEKRRLNLVMPARFFPISTKYLPLDKGIKPYYPDNVVNHYFQTRHYLHTLWKAGILYKRETTRSASFCGSPYSWEQELHHGAFLDGPSRMGEESFHHQSSGIFSRPPVGSSIQSKHQKSRLGPQSQQRPLDRSQQGRSGSIRAGVHSPTRRPFGVEPSGSRHAKNIASRSASCLHQSAVRKAAYPNHSTFERHSSSGHAVEFHNIPPSSAGSQSKRPVFSCWWLQFRNSEPCSDYCLTHLVNLLQDWGPCTEHGKHHIRIPRTPARVTGGVFLVDKNPHNTAESRLVVDFSQFSRGSSRVSWPKFAVPNLQSLTNLLSSNLSWLSLDVSAAFYHLPLHPAAMPHLLVGSSGLSRYVARLSSNSRIINHQYGTLPNLHDSCSRNLYISLMLLFKTFGRKLHLYSHPIIMGFRKIPMGVGLSPFLLAQFTSAICSVVRRAFPHCLAFSYMDDVVLGAKSVQHLESLYTAVTNFLLSLGIHLNPNKTKRWGYSLNFMGYVIGSWGSLPQEHIRLKIKDCFRKLPVNRPIDWKVCQRIVGLLGFAAPFTQCGYPALMPLYACTQSKQAFTFSPTYKAFLCKQYMNLYPVARQRPGLCQVFADATPTGWGLAIGHQRMRGTFVAPLPIHTAELLAACFARSRSGAKLIGTDNSVVLSRKYTSFPWLLGCAANWILRGTSFVYVPSALNPADDPSRGRLGISRPLLRLPFQPTTGRTSLYAVSPSVPSHLPDRVHFASPLHVAWRPP

SEQ ID NO: 42 - protein, 399 aa, Hepatitis B virus
MGLSWTVPLEWGKNHSTTNPLGFFPDHQLDPAFRANTRNPDWDHNPNKDHWTEANKVGVGAFGPGFTPPHGGLLGWSPQAQGMLKTLPANPPPASTNRQSGRQPTPITPPLRDTHPQAMQWNSTTFHQALQDPRVRGLYLPAGGSSSGTVNPVPTTASLISSIFSRIGDPAPNMEGITSGFLGPLLVLQAGFFLLTKILTIPQSLDSWWTSLNFLGGAPVCLGQNSQSPISNHSPTSCPPICPGYRWMCLRRFIIFLFILLLCLIFLLVLLDYQGMLPVCPLIPGSSTTSTGPCRTCTTLAQGTSMFPSCCCLKPSDGNCTCIPIPSSWAFGKFLWEWASARFSWLSLLVPFVQWFAGLSPTVWLSVIWMMWYWGPSLYNILSPFIPLLPIFFCLWVYI

SEQ ID NO: 43 - protein, 154 aa, Hepatitis B virus
MAARMCCQLDPARDVLCLRPVSAESCGRPVSGSLGDLSSPSPSAVPADHGAHLSLRGLPVCAFSSAGPCALRFTSARRMETTVNAHQILPKVLHKRTLGLSAMSTTDLEAYFKDCLFKDWEELGEEIRLKVFVLGGCRHKLVCAPAPCNFFTSA

SEQ ID NO: 44 - protein, 212 aa, Hepatitis B virus
MQLFHLCLIIFCSCPTVQASKLCLGWLWGMDIDPYKEFGASVELLSFLPSDFFPSVRDLLDTASALYRDALESPEHCTPNHTALRQAILCWGELMTLASWVGNNLEDPAARDLVVNYVNTNMGLKIRQLLWFHISCLTFGRETVLEYLVSFGVWIRTPPAYRPPNAPILSTLPETTVVRRRGRSPRRRTPSPRRRRSQSPRRRRSQSPASKC

SEQ ID NO: 45 - protein, 843 aa, Hepatitis B virus
MPLSYPHFRKLLLLDDEAGPLEEELPRLADEGLNRRVAEDLNLQLPNVSIPWTHKVGNFTGLYSSTVPTFNPDWLTPSFPDIHLHQDLIHKCEQFVGPLTKNELRRLKLVMPSRFFPKVTKYFPMEKGIKPYYPDNVVNHYFKTRHYLHTLWKAGILYKRESTRSASFCGSPYSWEQELQHGSTSINDSKGHGTESLCTQSSGILSRPSAGSSIQGKFQQSRLGLQQKQGQLANGKQGRSGRIRSWVHTPTRWPVGVESTGTGCAYNIASRSASCFHQSAVREKTNPSLSTSKRHSSTGHAVELHSVPPGSVRSEGKGSVFSCWWLQFRDTEPCSDYCLSHIINLLEDWGPCYEHGQHHIRTPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSTSRIHDHQHGTMQNLHNSCSRNLYVSLLLLFQTFGRKLHLYSHPIILGFRKIPMGVGLSPFLLAQFTSAICSVVRRAFPHCLAFSYMDDLVLGAKSVQHLESLYTAVTNFLLSVGIHLNTSKTKRWGYTLNFMGYVIGSWGSLPQDHIVQKLKDCFRKLPVNRPIDWKVCQRIVGLLGFAAPFTQCGYPALMPLYACITAKQAFVFSPTYKAFLCQQYMNLYPVARQRPGLCQVFADATPTGWGLAIGHQRMRGTFVAPLPIHTAELLAACFARSRSGAKLIGTDNSVVLSRKYTSFPWLLGCAANWILRGTSFVYVPSALNPADDPSRGRLGLYRPLLRLPFQPTTGRTSLYAASPSVPSHLPDRVHFASPLHVAWRPP

SEQ ID NO: 46 - protein, 400 aa, Hepatitis B virus
MGAPLSTTRRGMGQNLSVPNPLGFFPDHQLDPLFKANSSSPDWDFNKNKDTWPMANKVGVGAYGPGFTPPHGGLLGWSPQAQGVLTTLPADPPPASTNRRSGRKPTPVSPPLRDTHPQAMQWNSTQFHQALLDPRVRALSPPAGGSSSETQNPAPTIASLTSSIFSKTGGPAMNMDNITSGLLGPLLVLQAVCFLLTKILTIPQSLDSWWTSLNFLGGLPGCPGQNSQSPTSNHLPTSCPPTCPGYRWMCLRRFIIFLFILLLCLIFLLVLLDYQGMLPVCPLIPGSTTTSTGPCKTCTTLAQGTSMFPSCCCSKPSDGNCTCIPIPSSWALGKYLWEWASARFSWLSLLVQFVQWCVGLSPTVWLLIIWMIWYWGPNLCSILSPFIPLLPIFCYLWVSI

SEQ ID NO: 47 - protein, 154 aa, Hepatitis B virus
MAARLCCQLDPTRDVLCLRPVGAESRGRSLSGSLGAVPPPSPSAVPANDGSHLSLRGLPVCSFSSAGPCALRFTSARRMETTVNAPRSLPTVLHKRTLGLSGRSMTWIEDYIKDCVFKDWEELGEEIRLKVFVLGGCRHKLVCSPAPCNFFTSA

SEQ ID NO: 48 - protein, 194 aa, Hepatitis B virus
MDRTTLPYGLFGLDIDPYKEFGATVELLSFLPSDFFPSVRDLLDTASALYRESLESSDHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRGRSPRRRTPSPRRRRSQSPRRRRSASPASQ

SEQ ID NO: 49 - protein, 842 aa, Hepatitis B virus
MPLSYQHFRRLLLLDEEAGPLEEELPRLADEDLNRRVAEDLHLQLPNDSIPWTHKVGNFTGLYSSTIPVFNPDWQTPSFPNIHLHQDIITKCEQFVGPLTVNEKRRLKLVMPARFFPNSTKYLPLDKGIKPYYPENVVNHYFQTRHYLHTLWKTGILYKRETSRSASFCGSPYTWEQDLQHGAFLDGPSRVGKEPFHQQSSRIPSRSPVGPSIQSKYQQSRLGLQSQKGPLARGQQGRSWSLWTRVHPSTRRPFGVEPSVSGHTNNFASRSASCLHQSSVREAAYSHLSTTKRQSSSGHAVELYSIPPSSTKSQSQGPVSSCWWLQFRDSEPCSDYCLSHLVNLLQDWGPCTEHGEHHIRIPRTPARVTGGVFLVDKNPHNTAESRLVVDFSQFSRGSARVSWPKFAVPNLQSLTNLLSSNLSWLSLDVSAAFYHIPLHPAAMPHLLVGSSGLSRYVARLSSDSRILDHQYGTLQNLHDSCSRQLYVSLMLLYKTFGRKLHLYSHPIILGFRKIPMGLGLSPFLMAQFTSAICSVVRRAFPHCLAFSYVDDVVLGAKSVQHLESLYTAVTNFLLSLGIHLNPNKTKRWGYSLNFMGYVIGSWGTLPQEHITQKIKQCFRKLPVNRPIDWKVCQRITGLLGFAAPFTQCGYPALMPLYACIQAKQAFTFSPTYKAFLCKQYMNLYPVARQRPGLCQVFADATPTGWGLAIGHQRMRGTFVAPLPIHTAELLAACFARSRSGAKLIGTDNSVVLSRKYTSFPWLLGCAANWILRGTSFVYVPSALNPADDPSRGRLGLYRPLLRLPFLPTTGRTSLYAVSPSVPSHLPDRVHFASPLHVTWKPP

SEQ ID NO: 50 - protein, 399 aa, Hepatitis B virus
MGLSWTVPLEWGKNLSTSNPLGFLPDHQLDPAFRANTNNPDWDFNPKKDPWPEANKVGVGAYGPGFTPPHGGLLGWSPPSQGTLTTLPADPPPASTNRQSGRQPTPISPPLRDSHPQAMQWNSTAFHQALQNPKVRGLYLPAGGSSSGIVNPVPTIASHISSIFSRIGDPAPNMENITSGFLGPLLVLQAGFFLLTRILTIPQSLDSWWTSLNFLGGVPVCPGLNSQSPTSNHSPISCPPTCPGYRWMCLRRFIIFLFILLLCLIFLLVLLDYQGMLPVCPLIPGSSTTSTGPCKTCTTPAQGNSMYPSCCCTKPSDGNCTCIPIPSSWAFAKYLWDWASVRFSWLSLLVPFVQWFVGLSPTVWLSAMWMMWYWGPNLYNILSPFIPLLPIFFCLWVYI

SEQ ID NO: 51 - protein, 154 aa, Hepatitis B virus
MAARLCRQLDPSRDVLCLRPVSAESSGRPLPGPFGALSPPSPSAVPADHGAHLSLRGLPVCSFSSAGPCALRFTSARYMETAMNTSHHLPRQLYKRTLGLFVMSTTGVEKYFKDCVFAEWEELGNESRLMTFVLGGCRHKLVCAPAPCNFFTSA

SEQ ID NO: 52 - protein, 212 aa, Hepatitis B virus
MSLFHLCLIIFCSCPTVQASKLCLGWLWGMDIDPYKEFGASAELLSFLPSDFFPSVRDLLDTASALYREALESPEHCTPNHTALRQAILCWGELMTLASWVGNNLQDPAARDLVVNYVNTNMGLKIRQLLWFHISCLTFGRETVLEYLVSFGVWIRTPPAYRPPNAPILSTLPETTVVRQRGRAPRRRTPSPRRRRSQSPRRRRSQSPASQC

SEQ ID NO: 53 - protein, 843 aa, Hepatitis B virus
MPLSYQHFRRLLLLDNEAGPLEEELPRLADEDLNLRVAEDLNLQLPNVSIPWTHKVGNFTGLYSSTIPVFNPDWLTPSFPDIHLHQDLIQKCEQFVGPLTTNERRRLKLIMPARFYPKVTKYFPLDKGIKPYYPENVVNHYFKTRHYLHTLWKAGILYKRESTHSASFCGSPYSWEQELQHGSTSLNGEKGHGTESFCAQSSGILSRPPVGSTIQSKFQQSRLGLQHKQGQLANGKQGRSGRLRSRVHTPTRWPSGVEPSGTGHSNNLATRSTSCFHQSEVREKANPSLSTSKGHTSTGHAVELNTVPPSTVGSESKGAVSSCWWLQFRNTEPCSDYCLSHIINLLEDWGPCYEHGEHHIRTPRTPSRVTGGVFLVDKNPHNTKESRLVVDFSQFSRGTTRVSWPKFAVPNLQSLTNLLSSNLSWLSLDVSAAFYHLPLHPAAMPHLLVGSSGLSRYVARVSSTSRIYNHQHGTLQNLHHSCSRNLYVSLLLLYQTFGRKLHLYSHPIILGFRKIPMGVGLSPFLLAQFTSAICSVVRRAFPHCLAFSYMDDLVLGAKSVQHLESLYTAVTNFLLSVGIHLNTAKTKWWGYSLHFMGYVIGSWGTLPQEHIVHKIKDCFRKLPVNRPIDWKVCQRIVGLLGLAAPFTQCGYPALMPLYACITAKQAFVFSPTYKAFLCKQYMNLYPVARQRPGLCQVFADAPPTGWGLAIGHQRMRGTFVAPLPIHTAELLAACFARSRSGADIIGTDNSVVLSRKYTSFPWLLGCAANWILRGTSFVYVPSALNPADDPSRGRLGLCRPLLRLPFRPTTGRTSLYADSPPVPSHLPARVHFASPLHVAWRPP

SEQ ID NO: 54 - protein, 400 aa, Hepatitis B virus
MGAPLSTARRGMGQNLSVPNPLGFFPDHQLDPLFRANSSSPDWDFNTNKDNWPMANKVGVGGFGPGFTPPHGGLLGWSPQAQGILTTSPPDPPPASTNRRSGRKPTPVSPPLRDTHPQAMQWNSTQFHQALLDPRVRGLYLPAGGSSSETQNPAPTIASLTSSIFSKTGDPAMNMENITSGLLGPLLVLQAVCFLLTKILTIPKSLDSWWTSLNFLGVPPGCPGQNSQSPISNHLPTSCPPTCPGYRWMCLRRFIIFLFILLLCLIFLLVLLDYQGMLPVCPLLPGSTTTSTGPCKTCTTLAQGTSMFPSCCCTKPSDGNCTCIPIPSSWAFGKYLWEWASARFSWLSLLVQFVQWCVGLSPTVWLLVIWMIWYWGPNLCSILSPFIPLLPIFCYLWASI

SEQ ID NO: 55 - protein, 154 aa, Hepatitis B virus
MAARMCCQLDPARDVLCLRPVGAESCGRPLSWSLGALPPSSPPAVPADDGSHLSLRGLPACAFSSAGPCALRFTSARRMETTVNAPWNLPTTLHKRTLGLSPRSTTWIEEYIKDCVFKDWEESGEELRLKVFVLGGCRHKLVCSPAPCNFFTSA

(19) United States
(12) Patent Application Publication
(10) Pub. No.: US 2013/0121964 A1
(43) Pub. Date: May 16, 2013

(54) Pharmacogenomic and Response-Guided Treatment of Infectious Disease Using Yeast-Based Immunotherapy

(71) Applicants: David Apelian, Boonton Township, NJ (US); Alex Franzusoff, Nahant, MA (US)

(72) Inventors: David Apelian, Boonton Township, NJ (US); Alex Franzusoff, Nahant, MA (US)

(73) Assignee: GLOBEIMMUNE, INC., Louisville, CO (US)

(21) Appl. No.: 13/635,254

(22) PCT Filed: Mar. 14, 2011
(86) PCT No.: PCT/US11/28359

§ 371 Date: Jan. 28, 2013

Related U.S. Application Data
(60) Provisional application No. 61/313,774, filed on Mar. 14, 2010.
(60) Provisional application No. 61/313,776, filed on Mar. 14, 2010.
(60) Provisional application No. 61/313,775, filed on Mar. 14, 2010.
(60) Provisional application No. 61/370,899, filed on Aug. 5, 2010.
(60) Provisional application No. 61/407,859, filed on Oct. 28, 2010.

(51) Int. Cl. A61K 39/12; A61K 31/7056; A61K 38/21

(52) U.S. Cl. CPC A61K 39/12; A61K 38/21; A61K 38/212; A61K 31/7056
USPC 424/85.7; 424/204.1; 424/85.4; 424/186.1

(57) ABSTRACT

Disclosed are improved methods for treating an infectious disease with yeast-based immunotherapy, including viral disease, such as disease resulting from hepatitis virus infection, using a pharmacogenomic and response-guided approach based on IL28B genotype of the individual.

### CROSS-REFERENCE TO RELATED APPLICATIONS

This application claims the benefit of priority under 35 U.S.C. §119(e) to each of the following provisional applications, each of which is incorporated herein by reference in its entirety: U.S. Provisional Application No. 61/313,774, filed Mar. 14, 2010; U.S. Provisional Application No. 61/313,775, filed Mar. 14, 2010; U.S. Provisional Application No. 61/313,776, filed Mar. 14, 2010; U.S. Provisional Application No. 61/370,899, filed Aug. 5, 2010; and U.S. Provisional Application No. 61/407,859, filed Oct. 28, 2010.

### REFERENCE TO A SEQUENCE LISTING

This application contains a Sequence Listing submitted electronically as a text file by EFS-Web. The text file, named “3923-29-PCT_ST25”, has a size in bytes of 211 KB, and was recorded on 10 Mar. 2011. The information contained in the text file is incorporated herein by reference in its entirety pursuant to 37 CFR §1.52(e)(5).

### FIELD OF THE INVENTION

The present invention generally relates to improved methods for treating an infectious disease with yeast-based immunotherapy, including viral disease, such as disease resulting from hepatitis virus infection.

### BACKGROUND OF THE INVENTION

Immunotherapeutic compositions, including vaccines, are one of the most cost-effective measures available to the health care industry for the prevention and treatment of disease. There remains, however, an urgent need to develop safe and effective immunotherapy strategies for a variety of diseases, including those caused by or associated with infection by pathogenic agents. For the treatment of many infectious diseases, including viral diseases such as those caused by hepatitis virus infection, it is desirable to provide immunotherapy that elicits a cell-mediated (cellular) immune response. Diseases often vary in duration, symptoms, severity and/or outcome among particular individuals or patient populations, and various disease therapies are sometimes more successful in certain individuals or patient populations than in others. Therefore, there is a need to improve disease therapy by using information that is specific to an individual patient or patient population, i.e., personalized therapy, in order to optimize the patient's opportunity for successful treatment and/or to avoid or modify treatments that are unlikely to provide a benefit to the patient.

Two infectious agents, hepatitis C virus (HCV) and hepatitis B virus (HBV), are major causative agents of acute and chronic hepatitis worldwide. HCV infection affects more than 200 million people worldwide and represents a significant health problem in many countries (Lauer and Walker, N Engl J Med 2001; 345: 41-52; Shepard et al., Lancet Infect Dis 2005; 5:558-567.). Similarly, HBV has caused epidemics in various parts of the world, and is endemic in China (Williams, Hepatology, 2006, 44 (3): 521-526). About a third of the world's population, more than 2 billion people, have been infected with HBV, including at least 350 million chronic carriers of the virus.

Approximately 20-40% of individuals infected with HCV clear the virus during the acute phase, whereas the remaining 60-80% develop chronic disease which may result in hepatic failure and liver cancer (Villano et al., Hepatology 1999; 29:908-914; Seeff, Hepatology 2002; 36:S35-S46; Cox et al., Clin Infect Dis 2005; 40:951-958). There is at present no preventative composition for HCV infection, and therapeutic options are currently limited to Standard Of Care (SOC) interferon/ribavirin therapy, which is often poorly tolerated, is contraindicated in many subjects, and is expensive. In addition, the efficacy of the current standard treatment with interferon (interferon-α, including pegylated interferon-α) and ribavirin is limited, especially in genotype 1, the most prevalent genotype in the U.S. and most industrialized countries (Dienstag and McHutchison, Gastroenterology 2006; 130:231-264). Thus, only a proportion of HCV-infected persons can be successfully treated using current standard of care regimens.

Moreover, it has been shown that different patient groups respond differently to SOC regimens based on factors such as age, body mass index (BMI), viral load, gender, and race (Walsh et al., 2006, Gut 55, 529-535; Gao et al., 2004, Hepatology 39, 880-890; McHutchison, et al., 2009, NEJM 361:580-593; Fried, et al., 2002, NEJM 347:975-982). In addition, it has recently been shown that a genetic polymorphism upstream of the IL28B gene (which encodes interferon-λ) that is significantly associated with response to SOC therapy (pegylated interferon and ribavirin) for patients with chronic genotype 1 HCV infection (Ge et al., 2009, Nature 461, 399-401; Tanaka et al., 2009, Nature Genetics 41:1105; Suppiah et al., 2009, Nature Genetics 41:1100) as well as with the spontaneous clearance of HCV by individuals with acute infection (Thomas et al., 2009 Nature 461, 798-801). The polymorphism, designated rs12979860, correlates significantly with observed differences in response to SOC between European-American and African-American patients with chronic genotype 1 HCV infection (Ge et al., 2009, supra). More specifically, individuals fall into one of three genotypes at the rs12979860 locus: C/C (homozygous for the C allele), C/T (heterozygous for C and T alleles), or T/T (homozygous for the T allele). With respect to interferon therapy studies described above for HCV infection, C/C individuals have the greatest likelihood of achieving a complete response to standard of care interferon therapy, whereas response rates in C/T individuals are much poorer, and in T/T individuals are quite poor. The studies by Thomas et al. showed that this polymorphism was also associated with the spontaneous clearance of HCV by individuals with acute infection in a similar manner (i.e., C/C individuals have the greatest likelihood of spontaneously clearing acute infection, while spontaneous clearance in C/T individuals is much lower and in C/T individuals, lower still). Therefore, certain groups of patients (C/Ts and especially T/Ts) are predicted to have a poor outcome in current SOC therapy and spontaneous clearance of acute HCV infection based on their unfavorable IL28B genotype. A more recent study of patients chronically infected with genotype 2/3 HCV showed a similar association of the IL28B polymorphism with predicted outcome to SOC (Mangia et al., 2010, Gastroenterol. 139(3):821-827). Mangia et al. also suggested that, as in genotype 1 infections, the C/C genotype was more likely to be associated with spontaneous clearance of acute infection in HCV genotype 2/3.

In addition to the rs12979860 polymorphism, which has the strongest association signal with the phenotype to date, several other closely correlated polymorphisms have been identified and associated with outcomes in spontaneous clearance of acute HCV infection and/or response to interferon-based therapy/SOC (e.g., rs28416813, rs8103142, rs8099917, rs12980275, rs7248668, rs11881222, or rs8105790, see Ge et al., supra, Suppiah et al., supra, Tanaka et al., supra, Rausch et al., Gastroenterology, 2010, 138:1338-45, and McCarthy et al., Gastroenterology, 2010, 138:2307-14). However, these are so tightly linked to the rs12979860 polymorphism that they have not been separated from the response phenotype discussed above.

With respect to HBV, approximately 90% of infants and 25%-50% of children aged 1-5 years will remain chronically infected with HBV (Centers for Disease Control and Prevention as of September 2010). Approximately 25% of those who become chronically infected during childhood and 15% of those who become chronically infected after childhood die prematurely from cirrhosis or hepatocellular carcinoma, and the majority of chronically infected individuals remain asymptomatic until onset of cirrhosis or end-stage liver disease (CDC as of September 2010). 1 million deaths per year worldwide (about 2000-4000 deaths per year in the U.S.) result from chronic HBV infection. Current standard of care (SOC) therapy for HBV infection includes primarily antiviral drugs, such as lamivudine (EPIVIR®), adefovir (HEPSERA®), tenofovir (VIREAD®), telbivudine (TYZEKA®) and entecavir (BARACLUDE®), and also include interferons (e.g., interferon-α2a and pegylated interferon-α2a (PEGASYS®) or pegylated interferon-α2b (PEGINTRON®)). These drugs, in particular the anti-virals, are typically administered for long periods of time (e.g., daily or weekly for up to one to five years or longer), and although they slow or stop viral replication, they typically do not provide a complete “cure” or eradication of the virus, as measured by seroconversion and remission. Interferon-based approaches are toxic, have modest remission rates and cannot be tolerated long term. Therefore, there is continued a need to find a therapy that increases the rate of seroconversion and cure of patients chronically infected with HBV.

Accordingly, while standard of care (SOC) therapy provides the best currently approved treatment for patients suffering from infectious diseases such as chronic HCV or chronic HBV, the significant adverse effects of the regimens can lead to noncompliance, dose reduction, and treatment discontinuation, combined with a percentage of patients who still fail to respond or sustain response to therapy. Therefore, there remains a need in the art for improved therapeutic treatments for infectious disease. Moreover, recent studies examining the effect of patient genotype and clinical characteristics on response outcomes indicate that it would be desirable to be able to provide a more personalized approach to the treatment of individuals, for example, by controlling or influencing the immune response elicited by treatment based on the immune status and/or genetic background of an individual with respect to a particular disease or condition at a given time point, in addition to providing flexible treatment protocols guided by the individual's response to the treatment.

### SUMMARY OF THE INVENTION

The present invention generally relates to method of using a yeast-based immunotherapy (or immunotherapy having similar properties) in a pharmacogenomic and response-guided approach to the treatment of infectious disease. Specifically, the present invention demonstrates that yeast-based immunotherapy can be used as a cornerstone component in a variety of combination therapies in pharmacogenomic and response-guided approaches for the treatment of infectious disease. While the embodiments in the summary below are illustrative of various embodiments of the invention, the invention is not limited to these embodiments, as other embodiments of the invention are described in the detailed description of the invention and examples described herein.

One embodiment of the invention relates to a method to treat chronic hepatitis C virus (HCV) infection, and/or to prevent, ameliorate or treat at least one symptom of chronic HCV infection, in an individual having an IL28B genotype of C/T or T/T. The method includes the step of administering to the individual a yeast-based immunotherapeutic composition comprising at least one HCV antigen or immunogenic domain thereof and one or both of at least one interferon and at least one anti-viral compound. The immunotherapeutic composition and the interferon and/or anti-viral compound are administered concurrently for a period of time that is longer than the period of time established as effective for the interferon and/or anti-viral compound in the absence of the yeast-based immunotherapy. In one aspect, the immunotherapeutic composition and the interferon and/or anti-viral compound are administered concurrently for at least several weeks longer than the period of time established as effective for the interferon and/or anti-viral compound in the absence of the yeast-based immunotherapy. In one aspect, the interferon and/or anti-viral compound are administered concurrently for at least 4 to 48 weeks longer than the period of time established as effective for the interferon and/or anti-viral compound in the absence of the yeast-based immunotherapy.

Another embodiment of the invention relates to a method to treat chronic hepatitis C virus (HCV) infection, and/or to prevent, ameliorate or treat at least one symptom of chronic HCV infection, in an individual having an IL28B genotype of C/T or T/T. The method includes administering to the individual a therapeutic protocol comprising yeast-based immunotherapeutic composition comprising at least one HCV antigen or immunogenic domain thereof and one or both of at least one interferon and at least one anti-viral compound. The virus level is monitored in the individual, and, when the individual first achieves viral negativity, the individual is treated for an additional 4 to 48 weeks with the therapeutic protocol.

Another embodiment of the invention relates to a method to treat chronic hepatitis C virus (HCV) infection, and/or to prevent, ameliorate or treat at least one symptom of chronic HCV infection, in an individual having an IL28B genotype of C/C. The method includes the step of administering to the individual a yeast-based immunotherapeutic composition comprising at least one HCV antigen or immunogenic domain thereof and one or both of at least one interferon and at least one anti-viral compound. The immunotherapeutic composition and the interferon and/or anti-viral compound are administered concurrently, wherein the interferon and/or anti-viral compound are administered at a reduced dose, reduced frequency, and/or for a shorter period of time than the protocol established as effective for the interferon and/or anti-viral compound, respectively, in the absence of the yeast-based immunotherapeutic composition.

Yet another embodiment of the invention relates to a method to treat chronic hepatitis C virus (HCV) infection, and/or to prevent, ameliorate or treat at least one symptom of chronic HCV infection, in an individual. The method includes the administration to an individual of: (a) a yeast-based immunotherapeutic composition comprising at least one HCV antigen or immunogenic domain thereof, wherein the immunotherapeutic composition elicits a T cell-mediated immune response against one or more HCV antigens; (b) pegylated interferon-α; and (c) ribavirin. When the individual has an IL28B genotype of C/C, the immunotherapeutic composition, the pegylated interferon-α, and the ribavirin are administered concurrently over a period of 48 weeks to interferon-naïve individuals and over a period of 72 weeks to non-responder individuals, except that the interferon and/or the ribavirin may optionally be administered in reduced dose, reduced frequency, or for a shorter period of time than the protocol established as effective for the interferon and/or ribavirin, respectively, in the absence of immunotherapy. When the individual has an IL28B genotype of C/T or T/T, the immunotherapeutic composition, the pegylated interferon-α, and the ribavirin are administered concurrently over a period of 48 weeks to interferon-naïve individuals, and over a period of 72 weeks to non-responder individuals, except that, if the individual having an IL28B genotype of C/T or T/T does not reach viral negativity within the first 12 weeks of the period, then the immunotherapeutic composition, the pegylated interferon-α, and the ribavirin are administered for a period greater than 48 weeks for interferon-naïve individuals and for a period greater than 72 weeks for non-responder individuals. In one aspect, the immunotherapeutic composition and the interferon and/or ribavirin are administered for at least several additional weeks. In one aspect, the interferon and/or ribavirin are administered for an additional at least 4 to 48 weeks.

Another embodiment of the invention relates to a method to treat hepatitis virus infection in an individual, comprising treating the individual with a therapeutic protocol comprising administration of: (a) a yeast-based immunotherapeutic composition comprising at least one hepatitis virus antigen or immunogenic domain thereof, wherein the immunotherapeutic composition elicits a T cell-mediated immune response against one or more hepatitis virus antigens; and (b) one or more agent selected from: an interferon, an anti-viral compound, a host enzyme inhibitor, and/or an immunotherapeutic composition other than the immunotherapeutic composition of (a). The therapeutic protocol is modified for individuals having an IL28B genotype of C/C by reducing the dose and/or frequency and/or period of time of administration of one or more of the agents of (b) as compared to the dose and/or frequency and/or period of time of administration established as effective for the agents of (b) in the absence of immunotherapy. The therapeutic protocol is modified for individuals having an IL28B genotype of C/T or T/T by monitoring the responsiveness of these individuals to the protocol and extending the period of time of administration of the protocol for those individuals who are slow responders to the protocol. In one aspect, the hepatitis virus is hepatitis C virus (HCV). In one aspect, the hepatitis B virus is (HBV).

Another embodiment of the invention relates to a method to treat chronic hepatitis B virus (HBV) infection, and/or to prevent, ameliorate or treat at least one symptom of chronic HBV infection, in an individual. The method includes administering to an individual: (a) a yeast-based immunotherapeutic composition comprising at least one HBV antigen or immunogenic domain thereof, wherein the immunotherapeutic composition elicits a T cell-mediated immune response against one or more HBV antigens; and (b) one or more agents selected from interferon, lamivudine, adefovir, tenofovir, telbivudine, and entecavir. The immunotherapeutic composition and the one or more agents are administered concurrently to individuals having an IL28B genotype of C/C until the individual reaches seroconversion, except that the agents of (b) may optionally be administered in reduced dose, reduced frequency, or for a shorter period of time than the protocol established as effective for the agents of (b) in the absence of immunotherapy, followed optionally, by an additional period of administration of the agents of (a) and/or (b) for 1 to 12 months. The immunotherapeutic composition and the one or more agents are administered concurrently to individuals having an IL28B genotype of C/T or T/T until the individual reaches seroconversion, followed by an additional period of administration of the agents of (a) and/or (b) for 1 to 12 months.

Another embodiment of the invention relates to a method to treat chronic hepatitis B virus (HBV) infection, and/or to prevent, ameliorate or treat at least one symptom of chronic HBV infection, in an individual having an IL28B genotype of C/T or T/T. The method includes administering to the individual a therapeutic protocol comprising a yeast-based immunotherapeutic composition comprising at least one HCV antigen or immunogenic domain thereof and an anti-viral compound. The individual is monitored for seroconversion, and, when the individual first achieves seroconversion, the individual is treated for an additional 6-12 months with the therapeutic protocol.

Yet another embodiment of the invention relates to a method to treat an infectious disease in an individual comprising treating the individual with a therapeutic protocol comprising administration of a yeast-based immunotherapeutic composition. In this embodiment, the IL28B genotype of the individual is determined prior to administering the protocol. The time of administration of the therapeutic protocol is lengthened for individuals having a genotype of IL28B C/T or T/T who first respond to the therapeutic protocol later than the average time period for response for all individuals or for individuals having an IL28B genotype of C/C. Additionally, or alternatively, the therapeutic protocol is modified for individuals having an IL28B genotype of C/C by reducing the dosage, duration of administration, or the frequency of administration of one or more agents in the therapeutic protocol other than the yeast-based immunotherapeutic composition.

Another embodiment of the invention relates to a method to improve treatment of an infectious disease in an individual, comprising: (a) detecting the IL28B genotype of the individual prior to treating the individual; (b) administering a yeast-based immunotherapeutic composition concurrently with additional agents for the treatment of the infectious disease to individuals having an IL28B genotype of C/T or T/T, wherein the period of time over which the yeast-based immunotherapeutic composition and additional agents are administered is lengthened as compared to the period of time over which the yeast-based immunotherapeutic composition and additional agents are administered to individuals having an IL28B genotype of C/C; and optionally or alternatively, (c) administering a yeast-based immunotherapeutic composition concurrently with additional agents for the treatment of the infectious disease to individuals having an IL28B genotype of C/C, wherein the therapeutic protocol is modified to reduce the dosage, duration of administration, or frequency of administration of the additional agents in the protocol, as compared to the dosage, duration of administration, or frequency of administration of the additional agents in the absence of yeast-based immunotherapy.

Another embodiment of the invention relates to a method to treat viremia in an individual, comprising treating the individual with a therapeutic protocol comprising administration of a yeast-based immunotherapeutic composition. The IL28B genotype of the individual is determined prior to administering the protocol, and the period of time of administration of the therapeutic protocol is extended for individuals having a genotype of IL28B C/T or T/T who first respond to the therapeutic protocol later than the average time period for response in all individuals or for individuals having an IL28B genotype of C/C.

Yet another embodiment of the invention relates to a method to treat viremia in an individual or population of individuals who has an IL28B genotype of C/T or T/T, comprising treating the individual or population of individuals having an IL28B genotype of C/T or T/T with a therapeutic protocol comprising administration of a yeast-based immunotherapeutic composition. The individual is monitored for responsiveness to the therapeutic protocol and, if the individual is a slow responder to the therapeutic protocol, is treated for a longer period of time than an individual with an IL28B C/C genotype.

Another embodiment of the invention relates to a kit, wherein the kit includes: (a) nucleotide primers and/or probes for the detection of an IL28B polymorphism in a DNA sample; and (b) a yeast-based immunotherapeutic composition comprising a heat-inactivated whole yeast that expresses an antigen from an infectious disease pathogen.

Yet another embodiment of the invention relates to the use of yeast-based immunotherapeutic composition in a method for the treatment of an infectious disease in a protocol that comprises: (a) detection of the IL-28B genotype of an individual; (b) administration of a yeast-based immunotherapeutic composition concurrently with additional agents for the treatment of the infectious disease to individuals having an IL28B genotype of C/T or T/T, wherein the period of time over which the yeast-based immunotherapeutic composition and additional agents are administered is lengthened as compared to the period of time over which the yeast-based immunotherapeutic composition and additional agents are administered to individuals having an IL28B genotype of C/C; and optionally or alternatively (c) administration of a yeast-based immunotherapeutic composition concurrently with additional agents for the treatment of the infectious disease to individuals having an IL28B genotype of C/C, wherein the therapeutic protocol is modified to reduce the dosage, duration of administration, or frequency of administration of the additional agents in the protocol, as compared to the dosage, duration of administration, or frequency of administration of the additional agents in the absence of yeast-based immunotherapy.

Another embodiment of the invention relates to a method to treat an infectious disease in an individual, comprising: (a) detecting the IL28B genotype of the individual prior to treating the individual; and (b) administering a yeast-based immunotherapeutic composition in conjunction with a therapeutic protocol for the infectious disease to individuals having an IL28B genotype of C/T or T/T.

Another embodiment of the invention relates to a method to treat an infectious disease, to improve treatment of an infectious disease, and/or to prevent, ameliorate or treat at least one symptom of the disease, in an individual or population of individuals who has an IL28B genotype of C/T or T/T. The method comprises treating the individual or population of individuals having an IL28B genotype of C/T or T/T with a therapeutic protocol comprising administration of a yeast-based immunotherapeutic composition, wherein the individual is monitored for responsiveness to the therapeutic protocol and, after the individual achieves a clinical milestone for the treatment, the individual is treated for an additional defined period of time with the therapeutic protocol.

Another embodiment of the invention relates to a method to treat viremia in an individual, comprising treating the individual with a therapeutic protocol comprising administration of a yeast-based immunotherapeutic composition. The IL28B genotype of the individual is determined prior to administering the protocol, and the period of time of administration of the therapeutic protocol is extended for individuals having a genotype of IL28B C/T or T/T who first respond to the therapeutic protocol later than the average time period for response in all individuals or for individuals having an IL28B genotype of C/C.

Another embodiment of the invention relates to the use of a yeast-based immunotherapeutic composition comprising at least one HCV antigen or immunogenic domain thereof in the preparation of a medicament for, or to treat chronic hepatitis C virus (HCV) infection, in a therapeutic protocol comprising: administering to an individual having an IL28B genotype of C/T or T/T the yeast-based immunotherapeutic composition and one or both of at least one interferon and at least one anti-viral compound. The immunotherapeutic composition and the interferon and/or anti-viral compound are administered concurrently for a period of time that is longer than the period of time established as effective for the interferon and/or anti-viral compound in the absence of the yeast-based immunotherapy. In one aspect, the immunotherapeutic composition and the interferon and/or anti-viral compound are administered concurrently for at least several weeks longer than the period of time established as effective for the interferon and/or anti-viral compound in the absence of the yeast-based immunotherapy. In one aspect, the interferon and/or anti-viral compound are administered concurrently for at least 4 to 48 weeks longer than the period of time established as effective for the interferon and/or anti-viral compound in the absence of the yeast-based immunotherapy.

Another embodiment of the invention relates to the use of a yeast-based immunotherapeutic composition comprising at least one HCV antigen or immunogenic domain thereof in the preparation of a medicament for, or to treat chronic hepatitis C virus (HCV) infection, in a therapeutic protocol comprising: administering to the individual having an IL28B genotype of C/T or T/T a therapeutic protocol comprising the yeast-based immunotherapeutic composition and one or both of at least one interferon and at least one anti-viral compound. The virus level is monitored in the individual, and, when the individual first achieves viral negativity, the individual is treated for an additional 4 to 48 weeks with the therapeutic protocol.

Another embodiment of the invention relates to the use of a yeast-based immunotherapeutic composition comprising at least one HCV antigen or immunogenic domain thereof in the preparation of a medicament for, or to treat chronic hepatitis C virus (HCV) infection, in a therapeutic protocol comprising: administering to the individual having an IL28B genotype of C/C the yeast-based immunotherapeutic composition and one or both of at least one interferon and at least one anti-viral compound. The immunotherapeutic composition and the interferon and/or anti-viral compound are administered concurrently, wherein the interferon and/or anti-viral compound are administered at a reduced dose, reduced frequency, and/or for a shorter period of time than the protocol established as effective for the interferon and/or anti-viral compound, respectively, in the absence of the yeast-based immunotherapeutic composition.

Another embodiment of the invention relates to the use of a yeast-based immunotherapeutic composition comprising at least one HCV antigen or immunogenic domain thereof in the preparation of a medicament for, or to treat chronic hepatitis C virus (HCV) infection, in a therapeutic protocol comprising: administering to the individual: (a) the a yeast-based immunotherapeutic composition; (b) pegylated interferon-α; and (c) ribavirin. When the individual has an IL28B genotype of C/C, the immunotherapeutic composition, the pegylated interferon-α, and the ribavirin are administered concurrently over a period of 48 weeks to interferon-naïve individuals and over a period of 72 weeks to non-responder individuals, except that the interferon and/or the ribavirin may optionally be administered in reduced dose, reduced frequency, or for a shorter period of time than the protocol established as effective for the interferon and/or ribavirin, respectively, in the absence of immunotherapy. When the individual has an IL28B genotype of C/T or T/T, the immunotherapeutic composition, the pegylated interferon-α, and the ribavirin are administered concurrently over a period of 48 weeks to interferon-naïve individuals, and over a period of 72 weeks to non-responder individuals, except that, if the individual having an IL28B genotype of C/T or T/T does not reach viral negativity within the first 12 weeks of the period, then the immunotherapeutic composition, the pegylated interferon-α, and the ribavirin are administered for a period greater than 48 weeks for interferon-naïve individuals and for a period greater than 72 weeks for non-responder individuals. In one aspect, the immunotherapeutic composition and the interferon and/or ribavirin are administered for at least several additional weeks. In one aspect, the interferon and/or ribavirin are administered for an additional at least 4 to 48 weeks.

Another embodiment of the invention relates to the use of a yeast-based immunotherapeutic composition comprising at least one hepatitis virus antigen or immunogenic domain thereof in the preparation of a medicament for, or to treat hepatitis virus infection, in a therapeutic protocol comprising: administering to the individual (a) the yeast-based immunotherapeutic composition, wherein the immunotherapeutic composition elicits a T cell-mediated immune response against one or more hepatitis virus antigens; and (b) one or more agent selected from: an interferon, an anti-viral compound, a host enzyme inhibitor, and/or an immunotherapeutic composition other than the immunotherapeutic composition of (a). The therapeutic protocol is modified for individuals having an IL28B genotype of C/C by reducing the dose and/or frequency and/or period of time of administration of one or more of the agents of (b) as compared to the dose and/or frequency and/or period of time of administration established as effective for the agents of (b) in the absence of immunotherapy. The therapeutic protocol is modified for individuals having an IL28B genotype of C/T or T/T by monitoring the responsiveness of these individuals to the protocol and extending the period of time of administration of the protocol for those individuals who are slow responders to the protocol. In one aspect, the hepatitis virus is hepatitis C virus (HCV). In one aspect, the hepatitis B virus is (HBV).

Another embodiment of the invention relates to the use of a yeast-based immunotherapeutic composition comprising at least one HBV antigen or immunogenic domain thereof in a medicament for, or to treat chronic hepatitis B virus (HBV) infection, in a therapeutic protocol comprising: administering to an individual: (a) yeast-based immunotherapeutic composition that elicits a T cell-mediated immune response against one or more HBV antigens; and (b) one or more agents selected from interferon, lamivudine, adefovir, tenofovir, telbivudine, and entecavir. The immunotherapeutic composition and the one or more agents are administered concurrently to individuals having an IL28B genotype of C/C until the individual reaches seroconversion, except that the agents of (b) may optionally be administered in reduced dose, reduced frequency, or for a shorter period of time than the protocol established as effective for the agents of (b) in the absence of immunotherapy, followed optionally, by an additional period of administration of the agents of (a) and/or (b) for 1 to 12 months. The immunotherapeutic composition and the one or more agents are administered concurrently to individuals having an IL28B genotype of C/T or T/T until the individual reaches seroconversion, followed by an additional period of administration of the agents of (a) and/or (b) for 1 to 12 months.

Another embodiment of the invention relates to the use of a yeast-based immunotherapeutic composition comprising at least one HBV antigen or immunogenic domain thereof in a medicament for, or to treat chronic hepatitis B virus (HBV) infection, in a therapeutic protocol comprising: administering to an individual having an IL28B genotype of C/T or T/T a therapeutic protocol comprising a yeast-based immunotherapeutic composition and an anti-viral compound. The individual is monitored for seroconversion, and, when the individual first achieves seroconversion, the individual is treated for an additional 6-12 months with the therapeutic protocol.

Yet another embodiment of the invention relates to the use of a yeast-based immunotherapeutic composition in a medicament for, or to treat an infectious disease, in a therapeutic protocol comprising: administration of a yeast-based immunotherapeutic composition, where the IL28B genotype of the individual is determined prior to administering the protocol. The time of administration of the therapeutic protocol is lengthened for individuals having a genotype of IL28B C/T or T/T who first respond to the therapeutic protocol later than the average time period for response for all individuals or for individuals having an IL28B genotype of C/C. Additionally, or alternatively, the therapeutic protocol is modified for individuals having an IL28B genotype of C/C by reducing the dosage, duration of administration, or the frequency of administration of one or more agents in the therapeutic protocol other than the yeast-based immunotherapeutic composition.

Yet another embodiment of the invention relates to the use of a yeast-based immunotherapeutic composition in a medicament for, or to improve treatment of an infectious disease in an individual, in a therapeutic protocol comprising: (a) detecting the IL28B genotype of the individual prior to treating the individual; (b) administering a yeast-based immunotherapeutic composition concurrently with additional agents for the treatment of the infectious disease to individuals having an IL28B genotype of C/T or T/T, wherein the period of time over which the yeast-based immunotherapeutic composition and additional agents are administered is lengthened as compared to the period of time over which the yeast-based immunotherapeutic composition and additional agents are administered to individuals having an IL28B genotype of C/C; and optionally or alternatively, (c) administering a yeast-based immunotherapeutic composition concurrently with additional agents for the treatment of the infectious disease to individuals having an IL28B genotype of C/C, wherein the therapeutic protocol is modified to reduce the dosage, duration of administration, or frequency of administration of the additional agents in the protocol, as compared to the dosage, duration of administration, or frequency of administration of the additional agents in the absence of yeast-based immunotherapy.

Yet another embodiment of the invention relates to the use of a yeast-based immunotherapeutic composition in a medicament for, or to treat viremia in an individual, where the therapeutic protocol comprises: administration of a yeast-based immunotherapeutic composition. The IL28B genotype of the individual is determined prior to administering the protocol, and the period of time of administration of the therapeutic protocol is extended for individuals having a genotype of IL28B C/T or T/T who first respond to the therapeutic protocol later than the average time period for response in all individuals or for individuals having an IL28B genotype of C/C.

Yet another embodiment of the invention relates to the use of a yeast-based immunotherapeutic composition in a medicament for, or to treat viremia in an individual, where the therapeutic protocol comprises: administration of a yeast-based immunotherapeutic composition to an individual having an IL28B genotype of C/T or T/T. The individual is monitored for responsiveness to the therapeutic protocol and, if the individual is a slow responder to the therapeutic protocol, is treated for a longer period of time than an individual with an IL28B C/C genotype.

Another embodiment of the invention relates to a kit, wherein the kit includes: (a) nucleotide primers and/or probes for the detection of an IL28B polymorphism in a DNA sample; and (b) a yeast-based immunotherapeutic composition comprising a heat-inactivated whole yeast that expresses an antigen from an infectious disease pathogen.

Yet another embodiment of the invention relates to the use of yeast-based immunotherapeutic composition in a method for the treatment of an infectious disease in a protocol that comprises: (a) detection of the IL-28B genotype of an individual; (b) administration of a yeast-based immunotherapeutic composition concurrently with additional agents for the treatment of the infectious disease to individuals having an IL28B genotype of C/T or T/T, wherein the period of time over which the yeast-based immunotherapeutic composition and additional agents are administered is lengthened as compared to the period of time over which the yeast-based immunotherapeutic composition and additional agents are administered to individuals having an IL28B genotype of C/C; and optionally or alternatively (c) administration of a yeast-based immunotherapeutic composition concurrently with additional agents for the treatment of the infectious disease to individuals having an IL28B genotype of C/C, wherein the therapeutic protocol is modified to reduce the dosage, duration of administration, or frequency of administration of the additional agents in the protocol, as compared to the dosage, duration of administration, or frequency of administration of the additional agents in the absence of yeast-based immunotherapy.

Another embodiment of the invention relates to a method to treat an infectious disease in an individual, comprising: (a) detecting the IL28B genotype of the individual prior to treating the individual; and (b) administering a yeast-based immunotherapeutic composition in conjunction with a therapeutic protocol for the infectious disease to individuals having an IL28B genotype of C/T or T/T.

Another embodiment of the invention relates to a method to treat an infectious disease, to improve treatment of an infectious disease, and/or to prevent, ameliorate or treat at least one symptom of the disease, in an individual or population of individuals who has an IL28B genotype of C/T or T/T. The method comprises treating the individual or population of individuals having an IL28B genotype of C/T or T/T with a therapeutic protocol comprising administration of a yeast-based immunotherapeutic composition, wherein the individual is monitored for responsiveness to the therapeutic protocol and, after the individual achieves a clinical milestone for the treatment, the individual is treated for an additional defined period of time with the therapeutic protocol.

In one aspect of any of the embodiments described anywhere herein, the interferon is a type I interferon, including without limitation, interferon-α. In any of the embodiments of the invention described above, or elsewhere herein where the interferon type is not specified, in one aspect, the interferon is pegylated interferon-α-2a or pegylated interferon-α-2b. In one aspect of any of the embodiments described herein, the interferon is not interferon-λ. In one aspect of any of the embodiments described herein, the interferon is interferon-λ. In one aspect of any of the embodiments described herein, the interferon is consensus interferon.

In any of the embodiments of the invention described above, or elsewhere herein where the anti-viral compound is not already specified, in one aspect, the anti-viral compound is ribavirin. In one aspect, anti-viral compounds include ribavirin and an HCV protease inhibitor. In one aspect, the anti-viral compounds include ribavirin and an HCV polymerase inhibitor. In one aspect, the anti-viral compounds include one or two HCV polymerase inhibitors.

In any of the above embodiments of the invention, in one aspect, the infectious disease is a viral disease. In one aspect, the infectious disease is hepatitis virus infection. In one aspect, the infectious disease is chronic hepatitis C virus infection. In one aspect, the infectious disease is hepatitis B virus infection. In one aspect, the immunotherapeutic composition comprises at least one antigen or immunogenic domain thereof, wherein the antigen is associated with or is from a pathogen that causes the infectious disease. In one aspect, the antigen is selected from the group consisting of: viral antigens, fungal antigens, bacterial antigens, helminth antigens, parasitic antigens, ectoparasite antigens, and protozoan antigens. In one aspect, the antigen is from a virus, including any virus associated with chronic infection. In one aspect, the virus includes, but is not limited to, adenoviruses, arena viruses, bunyaviruses, coronaviruses, coxsackie viruses, cytomegaloviruses, Epstein-Barr viruses, flaviviruses, hepadnaviruses, hepatitis viruses, herpes viruses, influenza viruses, lentiviruses, measles viruses, mumps viruses, myxoviruses, orthomyxoviruses, papilloma viruses, papovaviruses, parainfluenza viruses, paramyxoviruses, parvoviruses, picornaviruses, pox viruses, rabies viruses, respiratory syncytial viruses, reoviruses, rhabdoviruses, rubella viruses, togaviruses, varicella viruses, and T-lymphotrophic viruses. In one aspect, the antigen is from a hepatitis virus. In one aspect, the hepatitis virus is HCV or HBV. In one aspect, the antigen is from human immunodeficiency virus (HIV). In one aspect, the antigen is from an infectious agent from a genus selected from the group consisting of: Aspergillus, Bordatella, Brugia, Candida, Chlamydia, Coccidia, Cryptococcus, Dirofilaria, Escherichia, Francisella, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma, Vibriocholerae, and Yersinia. In one aspect, the antigen is from a bacterium from a family selected from the group consisting of: Enterobacteriaceae, Micrococcaceae, Vibrionaceae, Pasteurellaceae, Mycoplasmataceae, and Rickettsiaceae. In one aspect, the bacterium is of a genus selected from: Pseudomonas, Bordetella, Mycobacterium, Vibrio, Bacillus, Salmonella, Francisella, Staphylococcus, Streptococcus, Escherichia, Enterococcus, Pasteurella, and Yersinia.

In any of the embodiments of the invention related to hepatitis C virus infection described herein, in one aspect, the antigen is a fusion protein comprising HCV sequences, wherein the HCV sequences consist of between one and five HCV proteins and/or immunogenic domains thereof, wherein the HCV proteins are selected from the group consisting of: HCV Core (positions 1 to 191 of SEQ ID NO:20); HCV E1 envelope glycoprotein (positions 192 to 383 of SEQ ID NO:20); HCV E2 envelope glycoprotein (positions 384 to 746 of SEQ ID NO:20); HCV P7 ion channel (positions 747 to 809 of SEQ ID NO:20); HCV NS2 metalloprotease (positions 810 to 1026 of SEQ ID NO:20); HCV NS3 protease/helicase (positions 1027 to 1657 of SEQ ID NO:20); HCV NS4a NS3 protease cofactor (positions 1658 to 1711 of SEQ ID NO:20); HCV NS4b (positions 1712 to 1972 of SEQ ID NO:20); HCV NS5a (positions 1973 to 2420 of SEQ ID NO:20); and HCV NS5b RNA-dependent RNA polymerase (positions 2421 to 3011 of SEQ ID NO:20). In one aspect, the composition elicits an immune response against each of the HCV proteins or immunogenic domains thereof in the fusion protein.

In one aspect, the HCV sequences consist of an HCV NS3 protease sequence or at least one immunogenic domain thereof linked to an HCV Core sequence or at least one immunogenic domain thereof, wherein the HCV NS3 protease sequence lacks the catalytic domain of a natural HCV NS3 protease, wherein the composition elicits an HCV NS3-specific immune response and an HCV Core-specific immune response. In one aspect, the HCV NS3 protease consists of the 262 amino acids of HCV NS3 following the initial N-terminal 88 amino acids of the full-length NS3 protein (positions 1115 to 1376 with respect to SEQ ID NO:20). In one aspect, the HCV Core sequence consists of amino acid positions 2 through 140 of the full-length HCV Core sequence (positions 2 to 140, with respect to SEQ ID NO:20). In one aspect, the hydrophobic C-terminal sequence of the HCV Core is truncated. In one aspect, the fusion protein comprises or consists of SEQ ID NO:2.

In another aspect, the HCV sequences consist of a full-length, inactivated HCV NS3 protein, or at least one immunogenic domain thereof, wherein the composition elicits an HCV NS3-specific immune response. In one aspect, the HCV NS3 protein comprises a mutation at residue 1165 of the HCV polyprotein sequence, with respect to SEQ ID NO:20, that results in inactivation of the proteolytic activity of the protein. In one aspect, the fusion protein comprises or consists of SEQ ID NO:4.

In another aspect, the HCV sequences consist of an HCV E1 protein or at least one immunogenic domain thereof fused to an HCV E2 protein or at least one immunogenic domain thereof, wherein the composition elicits an HCV E1-specific immune response and an HCV E2-specific immune response. In one aspect, the HCV E1 protein is a full-length protein and wherein the HCV E2 protein is a full-length protein. In one aspect, the fusion protein comprises or consists of SEQ ID NO:12. In one aspect, the HCV E1 protein is a truncated E1 protein consisting of amino acids 1 to 156 of HCV E1 (positions 192 to 347, with respect to SEQ ID NO:20). In one aspect, the HCV E2 protein is a truncated E2 protein consisting of amino acids 1 to 334 of HCV E2 (positions 384 to 717, with respect to SEQ ID NO:20). In one aspect, the fusion protein comprises or consists of SEQ ID NO:6.

In another aspect, the HCV sequences consist of a transmembrane domain-deleted HCV NS4b protein or at least one immunogenic domain thereof, wherein the composition elicits an HCV NS4b-specific immune response. In one aspect, the transmembrane domain-deleted HCV NS4b protein consists of amino acids 1 to 69 of HCV NS4b (positions 1712 to 1780, with respect to SEQ ID NO:20) linked to amino acids 177 to 261 of HCV NS4b (positions 1888 to 1972, with respect to SEQ ID NO:20). In one aspect, the fusion protein comprises or consists of SEQ ID NO:8.

In one aspect, the HCV sequences consist of a truncated HCV Core protein or at least one immunogenic domain thereof fused to an HCV E1 protein with deleted transmembrane domain or at least one immunogenic domain thereof fused to an HCV E2 protein with deleted transmembrane domain or at least one immunogenic domain thereof, wherein the composition elicits an HCV Core-specific immune response, an HCV E1-specific immune response, and an HCV E2-specific immune response. In one aspect, the truncated HCV Core protein consists of positions 2 to 140 of HCV Core protein (positions 2 to 140, with respect to SEQ ID NO:20), wherein the HCV E1 protein with deleted transmembrane domain consists of positions 1 to 156 of HCV E1 protein (positions 192 to 347, with respect to SEQ ID NO:20), and wherein the HCV E2 protein with deleted transmembrane domain consists of positions 1 to 334 of HCV E2 protein (positions 384 to 717, with respect to SEQ ID NO:20). In one aspect, the fusion protein consists of SEQ ID NO:14.

In another aspect, the HCV sequences consist of inactivated HCV NS3 or at least one immunogenic domain thereof fused to HCV NS4a or at least one immunogenic domain thereof fused to HCV NS4b lacking a transmembrane domain or at least one immunogenic domain thereof, wherein the composition elicits an HCV NS3-specific immune response, an HCV NS4a-specific immune response, and an HCV NS4b-specific immune response. In one aspect, the HCV NS3 protein consists of positions 1 to 631 of HCV HS3 (positions 1027 to 1657, with respect to SEQ ID NO:20), wherein the serine at position 1165 with respect to SEQ ID NO:20 has been substituted with alanine, to inactivate the protease; wherein the HCV NS4a protein consists of positions 1 to 54 of the HCV NS4a protein (positions 635 to 691, with respect to SEQ ID NO:20); and wherein the HCV NS4b protein consists of positions 1 to 69 of HCV NS4b (positions 1712 to 1780, with respect to SEQ ID NO:20) fused to positions 177 to 261 of HCV NS4b (positions 1888 to 1972, with respect to SEQ ID NO:20). In one aspect, the fusion protein comprises or consists of SEQ ID NO:16.

In one aspect, the HCV sequences consist of an HCV NS5a protein or at least one immunogenic domain thereof fused to an HCV NS5b protein containing an inactivating deletion of NS5b C-terminus or at least one immunogenic domain thereof, wherein the composition elicits an HCV NS5a-specific immune response. In one aspect, the HCV NS5a protein consists of 1 to 448 of HCV NS5a (positions 1973 to 2420, with respect to SEQ ID NO:20); and wherein the HCV NS5b protein consists of positions 1 to 539 of HCV NS5b (positions 2421 to 2959, with respect to SEQ ID NO:20). In one aspect, the fusion protein comprises or consists of SEQ ID NO:18.

In one aspect of any of the embodiments related to hepatitis B virus infection described anywhere herein, the antigen is selected from the group consisting of surface protein (L, M and/or S and/or any one or combination of functional and/or immunological domains thereof); precore/core/e (precore, core, e-antigen, and/or any one or combination of functional and/or immunological domains thereof); polymerase (full-length, RT domain, TP domain and/or any one or combination of functional and/or immunological domains thereof); and X antigen (or any one or combination of functional and/or immunological domains thereof).

In one aspect of any of the embodiments of the invention described anywhere herein, the method or protocol further comprises administration of the yeast-based immunotherapeutic composition alone for a period of 4-12 weeks, prior to administration of other agents in the protocol.

In one aspect of any of the embodiments of the invention described anywhere herein, the immunotherapeutic composition elicits a CD8+ T cell response. In one aspect, the immunotherapeutic composition elicits a CD4+ T cell response. In one aspect, the immunotherapeutic composition has one or more of the following characteristics: (a) stimulates one or more pattern recognition receptors effective to activate an antigen presenting cell; (b) upregulates adhesion molecules, co-stimulatory molecules, and MHC class I and/or class II molecules on antigen presenting cells; (c) induces production of proinflammatory cytokines by antigen presenting cells; (d) induces production of Th1-type cytokines by T cells; (e) induces production of Th17-type cytokines by T cells; (f) reduces the numbers and/or functionality of regulatory T cells (Treg); and/or (g) elicits MHC Class I and/or MHC Class II, antigen-specific immune responses.

In one aspect of any of the embodiments of the invention described anywhere herein, the immunotherapeutic composition comprises an adjuvant.

In one aspect of any of the embodiments of the invention described anywhere herein, the immunotherapeutic composition comprises a biological response modifier.

In one aspect of any of the embodiments of the invention described anywhere herein, the yeast-based immunotherapeutic composition comprises a yeast vehicle, wherein the antigen or immunogenic domain thereof is expressed by, attached to, or mixed with the yeast vehicle. In one aspect, the antigen or immunogenic domain thereof is expressed by the yeast vehicle. In one aspect, the yeast vehicle is selected from the group consisting of: a whole yeast, a yeast spheroplast, a yeast cytoplast, a yeast ghost, and a subcellular yeast membrane extract or fraction thereof. In one aspect, the antigen or immunogenic domain thereof is expressed by the yeast vehicle. In one aspect, the yeast vehicle is selected from the group consisting of: a whole yeast and a yeast spheroplast. In one aspect, the yeast vehicle is a whole yeast. In one aspect, the yeast vehicle is a heat-inactivated yeast. In one aspect, the yeast vehicle is from Saccharomyces. In one aspect, the yeast vehicle is from Saccharomyces cerevisiae.

### BRIEF DESCRIPTION OF THE DRAWINGS

FIG. 1 is a schematic drawing showing the design of a phase 2 clinical trial combining immunotherapy with Standard of Care therapy for chronic HCV infection.

FIG. 2 is a bar graph showing the ITT (Intent To Treat) analysis for End of Treatment (ETR) responses in a phase 2 clinical trial for all patients (Overall), interferon-naïve patients (IFN-naïve), and patients who were previously non-responsive to interferon therapy (Non-Responders), including p-values determined by 2-sided Fisher's exact test, demonstrating that triple therapy improved ETR as compared to SOC alone.

FIG. 3 is a graph showing response kinetics for interferon-naïve subjects receiving triple therapy versus SOC alone, demonstrating that interferon-naïve subjects receiving triple therapy showed a 10% absolute improvement in SVR (Sustained Virologic Response) and a 21% relative improvement in SVR over interferon-naïve subjects receiving SOC alone. FIG. 3 also shows that more subjects receiving triple therapy and achieving viral negativity during the first 12 weeks of treatment (RVR) went on to achieve SVR than subjects receiving SOC alone and achieving viral negativity during the first 12 weeks of treatment.

FIG. 4 is a graph showing response kinetics for non-responder subjects receiving triple therapy versus SOC alone, demonstrating that non-responder subjects receiving triple therapy showed a 12% absolute improvement in SVR (Sustained Virologic Response) over non-responder subjects receiving SOC alone.

FIG. 5 is a bar graph summarizing data shown in FIGS. 2-4, comparing the virologic responses at ETR and SVR for interferon-naïve subjects, non-responder subjects, and all subjects, receiving triple therapy versus SOC alone.

FIG. 6 is a bar graph illustrating SVR rates according to IL28B genotype (C/C versus C/T versus T/T as compared to Overall) in interferon-naïve subjects receiving triple therapy versus SOC alone.

FIG. 7 is a bar graph summarizing viral clearance (ETR and SVR) by IL28B genotype in interferon-naïve subjects receiving triple therapy versus SOC alone.

FIG. 8 is a graph showing response kinetics for interferon-naïve subjects who have the IL28B C/C genotype (triple therapy versus SOC alone), demonstrating that more IL28B C/C subjects receiving triple therapy achieved SVR than subjects receiving SOC alone (74% vs. 65%). FIG. 5 also shows that more IL28B C/C subjects receiving triple therapy and achieving viral negativity during the first 12 weeks of treatment (RVR) went on to achieve SVR than subjects receiving SOC alone and achieving viral negativity during the first 12 weeks of treatment (83% vs. 63%).

FIG. 9 is a graph showing response kinetics for interferon-naïve subjects who have the IL28B C/T genotype (triple therapy versus SOC alone), demonstrating that more IL28B C/T subjects receiving triple therapy and achieving viral negativity during the first 12 weeks of treatment (RVR) went on to achieve SVR than subjects receiving SOC alone and achieving viral negativity during the first 12 weeks of treatment (90% vs. 69%). FIG. 6 also shows that more IL28B C/T subjects receiving triple therapy reach viral negativity at end of treatment than IL28B C/T subject receiving SOC alone (69% vs. 54%), and that subjects who reach first viral negativity later during treatment appear to be more likely to relapse post-treatment.

FIG. 10 is a graph showing response kinetics for interferon-naïve subjects who have the IL28B T/T genotype (triple therapy versus SOC alone), demonstrating that a significant percentage of IL28B T/T subjects receiving triple therapy achieved SVR where as no IL28B T/T subjects receiving SOC alone achieved SVR (60% vs. 0%). FIG. 7 also shows that while equal numbers of triple therapy and SOC alone IL28B T/T subjects achieved viral negativity during the first 12 weeks of treatment (RVR), only those receiving triple therapy went on to achieve SVR (50% vs. 0%). FIG. 7 also shows that IL28B T/T subjects receiving triple therapy continued to achieve viral negativity after the first 12 weeks of treatment, whereas no additional IL28B T/T subjects receiving SOC alone achieved viral negativity after the first 12 weeks of treatment.

FIG. 11 is a bar graph showing that at end of treatment, the group of interferon-naïve and non-responders on triple therapy had improved ALT normalization as compared to subjects receiving SOC alone (61% vs. 36%).

FIG. 12A shows that at end of treatment for interferon-naïve (IFN-naïve) subjects (48 weeks), triple therapy demonstrated an improvement in ALT normalization as compared to subjects receiving SOC alone (56% vs. 28%).

FIG. 12B shows that at end of treatment for Non-responder subjects (72 weeks), triple therapy demonstrated an improvement in ALT normalization as compared to subjects receiving SOC alone (28% vs. 7%).

FIG. 13 is a graph showing that at 24 weeks post-treatment (SVR24), interferon-naïve subjects who received triple therapy demonstrated a sustained improvement in ALT normalization as compared to subjects receiving SOC alone (42% vs. 21%).

FIG. 14 is a graph showing that HCV-specific T-cell responses were increased by up to 10-fold in IL28B T/T subjects receiving triple therapy compared to IL28B T/T subjects receiving SOC alone.

FIG. 15 is a bar graph showing categorical cellular immune responses by IL28B subgroups in interferon-naïve subjects receiving triple therapy as compared to interferon-naïve subjects receiving SOC alone, and indicating that triple therapy improves cellular immunity in IL28B T/T subjects.

FIG. 16 is a graph showing a representative example of the ELISpot response to overlapping, non-optimized HCV peptide pools for one IL28B T/T subject in the triple therapy arm of a phase 2 clinical trial.

FIG. 17 is a schematic drawing showing a proposed mechanism of action of immune clearance of HCV-infected hepatocytes in the setting of triple therapy versus SOC alone.

### DETAILED DESCRIPTION OF THE INVENTION

The present invention generally relates to improved methods for treating infectious disease, including without limitation viral infection such as chronic hepatitis virus infection, with immunotherapy (e.g., treatment of a disease or condition by administration of an immunotherapeutic composition, such as a yeast-based immunotherapeutic composition). Specifically, the invention provides novel uses of immunotherapy to treat infectious disease that considers and combines: (a) the genetic background of the individual and how this genetic background can influence and guide treatment decisions with immunotherapy, and (b) the actual response of the individual to treatment regimens that include the immunotherapy. The invention is useful for the immunotherapeutic treatment of chronic hepatitis virus infection, including both chronic hepatitis C virus (HCV) infection and chronic hepatitis B virus (HBV) infection, and is also useful for treating other infectious diseases with immunotherapy. Described herein are novel methods that combine “response-guided treatment” and “pharmacogenomic guided immunotherapy” (together, “pharmacogenomic/response-guided immunotherapy”), based in part on an individual's genotype at or linked to a genetic locus known as IL28B (which may also be denoted “IL-28B”) and the individual's predicted immune response based on this genotype (and/or based on other closely linked/related polymorphisms described herein), in combination with a flexible approach to treatment that is guided by the individual's response to therapy. The method of the invention: (a) optimizes an individual's opportunity for successful treatment and/or (b) avoids, modifies or eliminates treatments that are unlikely to provide a benefit to the individual or that are toxic to the individual. In particular, the discovery described herein reveals that one or more genetic polymorphisms, generally referred to herein as the IL28B genotype of an individual, influence how individuals and/or particular groups of individuals respond to yeast-based immunotherapy, and reveals that yeast-based immunotherapy (and other similar types of immunotherapy) can alter a response to standard of care therapy (e.g., interferon therapy, anti-viral therapy, or other conventional therapy for infectious agents). This altered response can benefit individuals who otherwise may not respond to standard of care therapy, and/or who may suffer from toxicities and/or side effects caused by therapy that does not include a yeast-based immunotherapy component. Indeed, yeast-based immunotherapy improves cure rates (or rates of successful treatment or therapeutic benefit) in such individuals and can reduce, diminish or eliminate the use of components of standard of care therapy that have undesirable side effects.

More specifically, the present invention is based on the discovery that individuals respond differently to yeast-based immunotherapy depending on the particular genotype of the individual at a genetic locus called IL28B, and furthermore, that the response of the various genotypes to immunotherapy in combination with conventional or approved therapy (i.e., standard of care or “SOC”) is different than the response of these genotypes to SOC alone (i.e., response to yeast-based immunotherapy is not predicted by the response of the individual to SOC, such as anti-viral or interferon-based therapy). In particular, the present invention demonstrates that yeast-based immunotherapy augments a productive (beneficial) response against chronic infection by HCV, regardless of IL28B genotype. However, this augmentation was especially pronounced in the most “unfavorable” IL28B genotypes (i.e., IL28B genotypes that are predicted to respond only moderately or poorly, and in any event less well than the “favorable” IL28B genotype, to current standard of care (SOC) therapy for chronic HCV). Specifically, it is demonstrated herein that the addition of immunotherapy to an SOC protocol for chronic hepatitis C virus infection improved the therapeutic responses regardless of IL28B genotype of the patient (i.e., responses were enhanced in some regard, as compared to SOC, in each of the C/C, C/T, and T/T genotypes). However, the effect of immunotherapy was greatest in patients with the poorest prognosis genotype (T/T) (“poorest” based on predicted response to SOC therapy).

Accordingly, the present invention describes the use of immunotherapy, and particularly yeast-based immunotherapy, as a cornerstone component of an overall treatment regimen for infectious disease (i.e., in combination with standard of care therapy and new therapies) wherein, in individuals having a “C” allele at the IL28B locus, and particularly C/C genotype individuals, the use of immunotherapy, in addition to enhancing therapeutic immune responses and improving therapeutic outcomes, can shorten the time of therapy and/or reduce the duration, dose and/or frequency of administration of one or more components in the therapeutic protocol (i.e., dose sparing), particularly those such as interferon and anti-virals that contribute to toxicities and other undesirable side effects of the treatment. Indeed, in some individuals, the addition of yeast-based immunotherapy to the protocol can also be used to eliminate one or more of the non-immunological components of the therapy. In individuals having a “T” allele at the IL28B locus, including both C/T and T/T individuals, and particularly in T/T individuals, the addition of such immunotherapy to the treatment regimen will enhance therapeutic immune responses and improving therapeutic outcomes, even rescuing individuals who are otherwise expected to fail or have poor responses to the regimen in the absence of immunotherapy. In these individuals, the invention contemplates a robust response guided therapy including immunotherapy that may lengthen duration of treatment or otherwise modify treatment using therapeutic milestones, with the goal of improving response rates in these individuals. Indeed, even while lengthening the duration of treatment in such regimens, the use of immunotherapy as described herein can also allow for the reduction in the duration, dose and/or frequency of administration of one or more components in the therapeutic protocol, or even elimination of a component with undesirable side effects (e.g., toxicity or resistance). With the addition of immunotherapy to treatment regimens for infectious disease, new combinations and dosing protocols of a variety of agents are possible (e.g., immunotherapy combined with one, two, three or more anti-virals with the elimination of interferon, immunotherapy combined with one or more anti-virals plus interferon, add-in of immunotherapy to a standard non-immunological regimen on a response-guided basis, etc.).

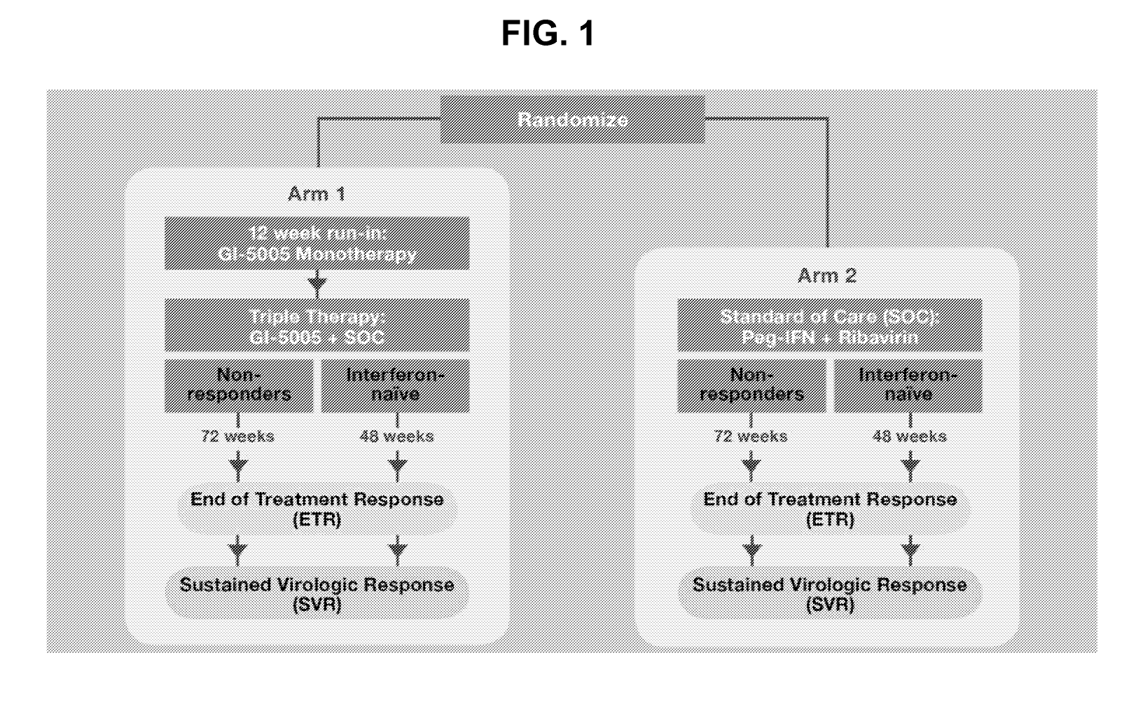

The discovery on which the present invention is based arose from a phase 2 clinical trial for the treatment of chronic hepatitis C virus infection, although the present invention can be extended to the treatment of HBV, as well as other infectious diseases that benefit, or could benefit, from immunotherapeutic approaches. In this phase 2 clinical study, yeast-based immunotherapy for chronic hepatitis C virus infection, denoted GI-5005, was used in combination with SOC (interferon and ribavirin) to treat individuals with chronic hepatitis C virus infection. More specifically, GI-5005, a whole, heat-killed S. cerevisiae immunotherapy product expressing high levels of HCV NS3 and Core antigens, was used in conjunction with pegylated-interferon-α and ribavirin (SOC) to treat subjects with genotype 1 chronic HCV infection (see FIG. 1 for schematic drawing of trial design). Patients (140 total enrolled) were randomized 1:1, and stratified by virologic response during their prior course of treatment in this open label trial. Arm 1 patients received a GI-5005 monotherapy run-in consisting of five weekly followed by 2 monthly subcutaneous (SC) doses of 40 YU (1 YU=10,000,000 yeast) GI-5005 over 12 weeks (administered as 10 YU doses to four separate sites on the patient), followed by triple therapy consisting of monthly 40 YU GI-5005 doses plus pegylated interferon and ribavirin (administered for 48 weeks in interferon-naïve patients and for 72 weeks in patients who were prior non-responders to interferon therapy). Arm 2 patients received treatment with SOC alone (without antecedent GI-5005 monotherapy). In results reported through end of treatment response (ETR), triple therapy (i.e., yeast-based immunotherapy in combination with SOC (pegylated interferon-α and ribavirin)) was shown to improve viral kinetics and improve complete response rates in various patient groups, as well as improve liver function and/or reduce liver damage, as compared to SOC alone (McHutchison et al. “GI-5005 Immunotherapy Plus Peg-IFN/Ribavirin In Genotype 1 Chronic Hepatitis C Patients Compared To Peg-IFN/Ribavirin Alone In Naive and Non-Responder Patients; Preliminary RVR and Viral Kinetic Analysis From the GI-5005-02 Phase 2 Study” Poster presentation; AASLD Nov. 3, 2008; Lawitz et al. “GI-5005 Immunotherapy Plus PEG-IFN/Ribavirin Versus PEG-IFN/Ribavirin in Genotype 1 Chronic HCV Subjects; Preliminary Phase 2 EVR Analyses” Poster presentation; EASL Apr. 24, 2009; McHutchison et al. “GI-5005 Therapeutic Vaccine Plus Peg-IFN/Ribavirin Improves End of Treatment Response at 48 Weeks Versus Peg-IFN/Ribavirin in Naïve Genotype 1 Chronic HCV Patients” Poster presentation; AASLD Nov. 2, 2009; and PCT Application No. PCT/US2009/057535).

Prior to the present invention, the impact of IL28B genotype on responsiveness to immunotherapy, including but not limited to yeast-based immunotherapy, was unknown. When the influence of IL28B on end of treatment response (ETR) and sustained virologic response (SVR) to triple therapy in naïve genotype-1 chronic HCV patients was assessed, it was discovered by the present inventors that the addition of immunotherapy to SOC (triple therapy) improves response rates (i.e., in HCV, viral negativity, which is defined elsewhere herein) in all IL28B genotypes. However, there were also differences in how each IL28B genotype responded to immunotherapy, each of which was of interest. The IL28B genotyping revealed an unexpected response correlation based on genotype, which further informed how patient responses can be improved using response guided therapy with immunotherapy as a cornerstone.

Specifically, patients receiving GI-5005 in conjunction with SOC who were also IL28B T/T genotype had best therapeutic responses of all patients having a T allele at the IL28B locus (C/Ts and T/Ts). In contrast, patients receiving only SOC (no yeast-based immunotherapy) had the worst responses of all patients having a T allele at the IL28B locus. Moreover, triple therapy utilizing yeast-based immunotherapy in combination with SOC improved HCV-specific immune responses, as compared to SOC, in all three genotypes (C/C, C/T and T/T), with the biggest difference observed in T/Ts. Moreover, the results showed that some patients, particularly those of the IL28B genotypes C/T and T/T, (i.e., a patient having a “T allele” at the IL28B locus that predicts poorer responses to SOC therapy), while being capable of responding to immunotherapy in a manner that is therapeutically beneficial (e.g., viral negativity for HCV patients), tended to respond more slowly than patients of the prognosis-favorable IL28B genotype (C/C), for example. This discovery indicated that the current guidelines for timing of therapy according to an SOC protocol, which can lead to decision points for patients based on a prior prediction of outcome, are not sufficient to predict outcome to immunotherapy. Indeed, the invention provides for the response-guided modification of treatment (e.g., by extension of the total period of treatment and/or modification of the agents used in the therapy) for individuals with slower responses to immunotherapy, in order to improve the individual's chance of having a complete response to the infection (e.g., achieving sustained viral negativity).

Accordingly, the present invention demonstrates that IL28B status is an indicator of how an individual is predicted to respond to immunotherapy, and provides a basis for novel strategies for treatment of infectious disease using immunotherapy, and specifically, yeast-based immunotherapy. While the results provided herein are shown in the context of chronic HCV infection and treatment with the exemplary SOC combination of interferon and ribavirin, because the benefits of the present invention represent a correlation between the addition of a particular type of immunotherapy to a treatment protocol and the IL28B genotype of a patient, the invention is readily adapted to the pharmacogenomic/response-guided treatment of any infectious disease using immunotherapy, and particularly, yeast-based immunotherapy. As discussed above, new combinations of improved anti-viral drugs administered with or without interferon therapy continue to improve response rates for patients chronically infected with HCV (e.g., TELAPREVIR™, an NS3 protease inhibitor from Vertex/Johnson & Johnson/Mitsubishi administered in combination with ribavirin and pegylated interferon-α; BOCEPREVIR™, an NS3 protease inhibitor from Merck & Co., Inc. administered in combination with ribavirin and pegylated interferon-α; PSI-7977, a uridine nucleotide analog polymerase inhibitor from Pharmasset, administered in combination with ribavirin with or without pegylated interferon-α; and combination PSI-7977 and PSI-938 (a guanine nucleotide analog polymerase inhibitor from Pharmasset), administered as a combination therapy without interferon). However, these drugs, like their predecessors, act directly on the virus without addressing the apparent differences in the immune responses generated by patients of different IL28B genotypes, and most still require administration with interferon, which causes some of the more severe toxicities. Indeed, even under new therapeutic regimens in development, there still appears to be a “tiered” rate of response based on IL28B genotype, with individuals having a T allele showing at least a trend toward responding less favorably (e.g., Jacobson et al., 2011, “Telaprevir substantially improved SVR rates across all IL28B genotypes in the ADVANCE trial”, EASL, Berlin, Germany, abstract; Pol et al., 2001, “Similar SVR Rates In IL28B CC, CT Or TT Prior Relapser, Partial- Or Null-Responder Patients Treated With Telaprevir/Peginterferon/Ribavirin: Retrospective Analysis Of The Realize Study”, EASL, Berlin, Germany, abstract; Poordad et al., 2001, “IL28B Polymorphism Predicts Virologic Response In Patients With Hepatitis C Genotype 1 Treated With Boceprevir (Boc) Combination Therapy”, EASL, Berlin, Germany, abstract).

In contrast, the yeast based immunotherapy of the present invention is uniquely poised to address the needs of an immune response that is not inherently able to combat a given infectious disease, or that would benefit from an enhancement of the immune response. Therefore, the addition of yeast-based immunotherapy as a cornerstone component in a variety of combination therapies based on interferon, anti-viral drugs, and other therapeutic approaches for infectious disease, will further improve response rates to therapy in all patients, including even the hardest to treat IL28B genotypes. Yeast-based immunotherapy is expected to “rescue” individuals for whom standard of care will continue to fail, and will allow the reduction, elimination, or shortened use of non-immunologic therapeutics and/or cytokines, most of which are associated with toxicities and other undesired side effects such as drug resistance, thereby improving patient compliance, quality of life, and chances for successful treatment.

As discussed above, the present invention is expected to have specific advantages for individuals carrying the IL28B T allele, and particularly, for those who are homozygous for the T allele. In addition, since some subpopulations of individuals carry the T allele at higher frequency, the present invention is expected to provide advantages to such subpopulations that have been historically more difficult to treat using SOC therapy. For example, Ge et al. supra, showed that African Americans have a higher incidence of the T/T genotype in the population, and Hispanics also show a higher frequency of this genotype, as compared to persons of European ancestry. Based on the prior studies of the IL28B genotype, which predicts a poor outcome to SOC for chronic HCV patients having the T/T genotype, it is possible that such individuals (those having the T/T genotype) would be dissuaded from pursuing SOC therapy altogether, and it is more likely that such individuals would be removed from SOC therapy early for failure to reach the early response endpoint(s), based on prior statistics that sustained virologic response (SVR) becomes much less likely for patients who are not at viral negativity by these timepoints. Moreover, even improved anti-viral therapies under development still appear to show the lowest response rates in patients having at least one T allele; as such therapeutic regimens further raise response rates for all patients, the patients who still fail to respond to the new regimens are even more likely to be left with no options for treatment. However, the present invention offers new hope for positive therapeutic outcomes in these patient groups with the poorest prognosis. Indeed, while the results of Ge et al. might have suggested that certain individuals (e.g., those with a T/T genotype) may not be good candidates for current HCV therapy, the present invention completely changes that prognosis—by the addition of immunotherapy to the protocol, such patients now may have a good prognosis for recovery. Extending this analysis to the findings that the T/T allele appears with higher frequency in certain racial groups, use of immunotherapy according to the present invention will provide substantial improvements in therapy in these populations as a whole. The present invention changes the course of such therapy to one that is personalized using objective molecular markers such as the IL28B genotype, and that can be modified based on the responsiveness of an individual to immunotherapy.

The data provided herein indicate that immunotherapy can change the way an individual is predisposed to respond to therapy for infectious disease, most likely by altering the immune response in the individual in a way that promotes reduction or alleviation of the infection or direct symptoms thereof. The IL28B gene encodes a type III interferon known as interferon-λ3. It is not currently known whether the rs12979860 polymorphism, or any closely correlated polymorphism (e.g., rs28416813, rs8103142, rs8099917, rs12980275, rs7248668, rs11881222, or rs8105790, see Ge et al., supra, Suppiah et al., supra, Tanaka et al., supra, Rausch et al., Gastroenterology, 2010, 138:1338-45, and McCarthy et al., Gastroenterology, 2010, 138:2307-14) are causally associated with the phenotype, or whether the phenotype occurs via an impact on interferon-λ. However, without being bound by theory, the present invention in one embodiment encompasses modification of therapeutic protocols for the treatment of infectious diseases based on effects in the regulation of or activity of one or more IFN-λ cytokines. There are three interferon-λ cytokines: interferon-λ3 (encoded by IL28B), interferon-λ1 (encoded by IL29), and interferon-λ2 (encoded by IL28A), and all three genes are located on chromosome 19. The class of interferon-λ proteins was first described in 2002 and published in 2003 (Kotenko et al., Nat. Immunol. 4:69-77 (2003); and Sheppard et al., Nat. Immunol. 4:63-68 (2003)). This class of interferons is structurally distinct from type I interferons, such as interferon-α, and utilizes a different receptor with different tissue distribution than type I interferons, but IFN-λs have similar signal transduction pathways and similar biological functions as type I interferons. The group of three cytokines is commonly referred to simply as interferon-λ. Interferon-λ, like type I interferons, is induced by viral infection, and has been shown to inhibit viral replication, including HCV replication, although it is believed that interferon-λ may actually be exerting influence on the immune system, rather than directly on the virus (see review by Uze et al., 2007, Biochimie 89:729-734). Indeed, interferon-λ1 (encoded by IL29) is currently in clinical trials for chronic HCV as a possible alternative to interferon-α. In addition, antiproliferative and apoptotic effects of type III interferon have been shown in vitro (Maher et al., Cancer Biol. Ther. 2008, 7, 1109-1115; Li et al., Cell Prolif. 2008, 41, 960-979) and anti-tumor effects have been shown in mice (Lasfar et al., Cancer Res. 2006, 66, 4468-4477; Sato et al. J. Immunol. 2006, 176, 7686-7694).

In 2009, Mennechet and Uze showed that interferon-λ treated dendritic cells (DCs) specifically induced the proliferation of a subset of T cells known as “regulatory T cells” or “Treg” (CD4+CD25+Foxp3+ T cells) (Mennechet and Uze, 2009, Blood 107(11):4417-4423). Mennechet and Uze therefore propose that interferon-λ may be involved in the active suppression of effector T cells by upregulation of Treg, which by extension, could act to downregulate the T cell responses that would be acting to eliminate HCV infected cells. Since the interferon-λ family members may have some opposite functions, modification of the function or expression of one or the other may contribute to different immune regulation in vivo.

Yeast-based immunotherapy is now known to upregulate a TH17 immune response, and to be able to suppress Treg numbers and/or functionality, which would theoretically oppose the action of the interferon-λ member proposed by Mennechet and Uze (unpublished data). Yeast-based immunotherapy may also enhance the action of other interferon-λ family members that may upregulate Treg action. In addition, yeast-based immunotherapy activates dendritic cells via a spectrum of “danger signals” mediated through pattern recognition receptors that are engaged by yeast. In the absence of such signaling to and activation of myeloid-derived antigen presenting cells or dendritic cells, such as might be expected by encounter of these cells with a different stimulus, such as cytokine alone (e.g., IFN-λ or IFN-α), these cells become myeloid-derived suppressor cells (MDSC) which suppress T cell responses (see, e.g., Yang et al., 2004, Nat. Immunol. 5(5):508-515; or Nagaraj et al., 2010, J. Immunol. 184:3106-3116).

In one aspect of the invention, if the polymorphism upstream of the IL28B gene (or a closely related polymorphism) is impacting IFN-λ function (which may include any one, two or three of the IFN-λ proteins, since the genes are clustered together), then without being bound by theory, the present invention provides insight that can modify how individuals are treated using interferon therapy, including without limitation both type I and type III interferons, and which individuals are treated using interferon therapy, and/or may indicate new protocols for modulating HCV infection using interferon therapy based on genotype. For example, and without being bound by theory, if the polymorphism in individuals carrying a T allele described herein somehow modifies IFN-λ (e.g., by changing the regulation of the production of the cytokine(s)), then it is conceivable that this modified IFN-λ production would be inhibiting a productive T cell response in individuals carrying a T allele, and the administration of immunotherapy as described herein relieves that inhibition and allows a productive immune response to occur. Accordingly, in one aspect, the present invention would provide for the use of immunotherapy in all patients, but may indicate that IFN-λ administration is not preferred, at least in certain subsets of patients, such as those carrying a T allele. Alternatively, IFN-λ administration together with immunotherapy may be contemplated if the action of the immunotherapy in combination with IFN-λ provides a therapeutic benefit. It is also contemplated by the invention that additional polymorphisms near or in the interferon-λ encoding genes may be contributing to the poorer outcomes observed in certain individuals in response to SOC therapy, and that the addition of immunotherapy to the protocol can overcome this phenotype and augment a beneficial response in such an individual.

Regardless of the mechanism of action, yeast-based immunotherapy, and immunotherapy that provides a similar type of immune response, provides a benefit to patients that appear to otherwise suffer from an immune deficiency, immune suppression, or simply an ineffective T cell activation pathway with respect to the ability to clear HCV, which can extend to other infectious diseases, perhaps by altering the manner in which T cells are activated and/or respond in an individual, or by providing an alternate pathway of activation of T cells in the individual. The use of yeast-based immunotherapy according to the present invention improves the ability of individuals to mount an effective immune response against infectious disease. While much of the discussion herein utilizes chronic HCV infection, current SOC for HCV, and the response of individuals to yeast-based immunotherapy in combination with SOC based on IL28B genotype, these scenarios are exemplary of the invention. Indeed, the invention is based on an understanding of how yeast-based immunotherapy acts on individuals of specific IL28B genotype, and since the effect is believed, without being bound by theory, to be related to the immune response elicited by the individual against an infectious disease, and since it is a function of the immune system to combat infectious disease, the invention is readily extended to the treatment of other infectious diseases and the corresponding conventional, or SOC, treatment for such diseases.

According to the present invention, “C/C” individuals, which are individuals having the C/C genotype at the IL28B locus (described in more detail below), are predicted to have the best prognosis for responding to conventional treatment regimens for HCV (e.g., SOC, such as combinations of direct-acting agents, such as anti-viral drugs and/or interferons). For example, in HCV, approximately 78% of C/C individuals will achieve sustained virologic response (SVR), which is indicative of “cure” in HCV, in response to SOC (Ge et al., supra). In addition, C/C individuals are the most likely to spontaneously clear an HCV infection (Thomas et al., supra). The inventors have now discovered how C/C individuals respond to yeast-based immunotherapy in the context of chronic HCV infection and ongoing SOC treatment, and provide herein a pharmacogenomic and response-guided approach to treatment of these individuals, which is readily expanded to other infectious conditions or diseases.

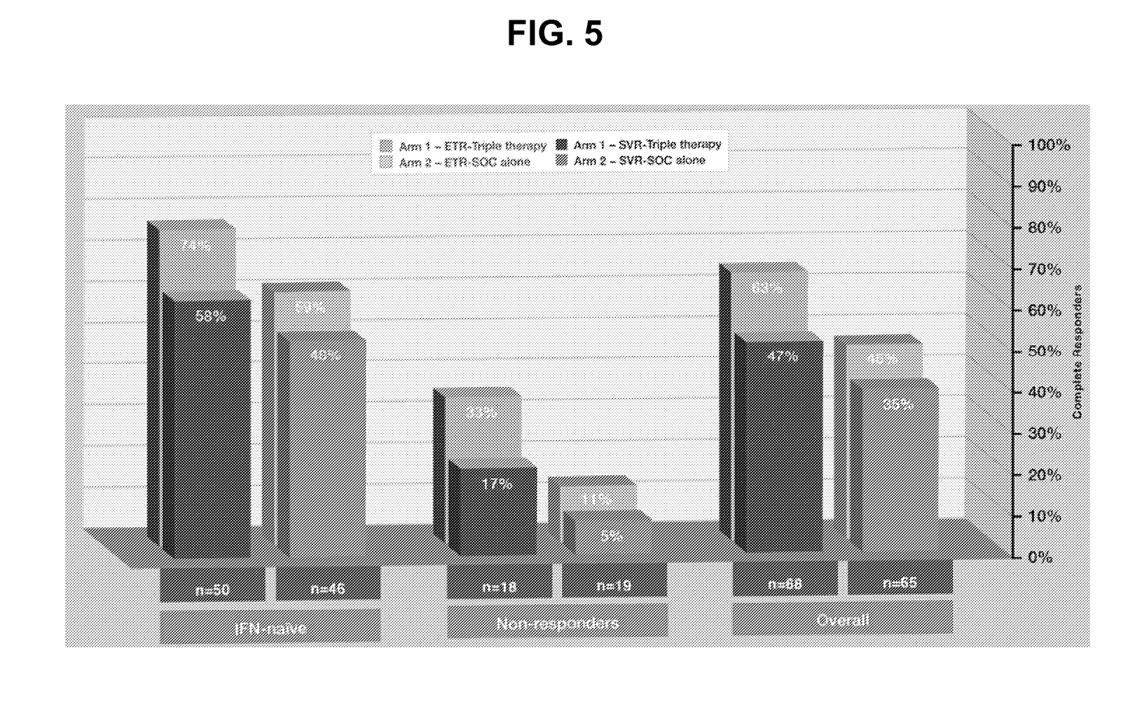

In the studies described herein, a substantial number of C/C individuals responded to SOC plus yeast-based immunotherapy (triple therapy) early in treatment (the first 12 weeks), and the same was true for C/C individuals receiving SOC alone; however, a greater percentage of C/C individuals receiving triple therapy who reached RVR or cEVR went on to achieve complete responses at the end of treatment and SVR, as compared to C/C individuals receiving SOC alone. Therefore, while C/C individuals generally respond well to both triple therapy and SOC alone and with similar kinetics, triple therapy delivered substantially more C/C patients to complete response by the ETR and SVR endpoints (see FIG. 5). Indeed, while both SOC and triple therapy C/C patients in the study described herein had good SVR rates overall, triple therapy still provided an advantage of almost 10%. Accordingly, yeast-based immunotherapy benefits IL28B C/C individuals by improving the likelihood that they will respond to therapy. Even though IL28B C/C individuals appear to inherently mount a more effective immune response against an infectious agent such as HCV than the other IL28B genotypes, adding yeast-based immunotherapy to standard of care regimens enhances the responses, allowing more IL28B C/C individuals to achieve successful therapeutic endpoints. Furthermore, identification of IL28B C/C individuals can allow such individuals to modify treatment to reduce toxicities and other side effects such as drug resistance, and can further reduce and length or duration of treatment with one or all of the agents when immunotherapy is added to the regimen. For example, yeast-based immunotherapy can allow IL28B C/C individuals to modify dosage of the other agents in the therapeutic regimen (e.g., reduce the duration, dose and/or frequency of one agent, particularly those with greater toxicity or other side effects), to modify the combination of agents administered (e.g., eliminate a toxic agent, such as interferon, and/or add a less toxic anti-viral), and/or to modify the total time of treatment (e.g., reduce the total time of treatment), in order to reduce side effects, reduce the likelihood of developing drug resistance, and improve patient compliance, without sacrificing therapeutic outcome. For example, IL28B C/C individuals may be able to reduce or eliminate the dosage and/or frequency of interferon-α and/or ribavirin and/or another anti-viral and/or other small molecule drugs (e.g., protease inhibitors) in the SOC component of therapy when using yeast-based immunotherapy to treat chronic HCV infection, and/or may be able to shorten the course of treatment by adding immunotherapy. In addition, yeast-based immunotherapy may rescue C/C patients who struggle to achieve a positive response in the absence of such therapy. Using the methods of the present invention, adding immunotherapy to SOC therapy is expected to produce a meaningful advantage in individuals having an IL28B C/C genotype as compared to SOC, even while using the same therapy protocol as for SOC (e.g., without extending the therapy period).

According to the present invention, “C/T” individuals, who are individuals having the heterozygous C/T genotype at the IL28B locus (described in detail below), are predicted to have a moderate prognosis of responding to conventional treatment regimens for HCV (e.g., SOC, such as combinations of direct-acting agents, such as anti-viral drugs and/or interferons). For example, in chronic HCV infection, approximately 37% of these individuals will achieve SVR in response to SOC therapy) (Ge et al., supra). The inventors have now discovered how C/T individuals respond to yeast-based immunotherapy in the context of chronic HCV infection and ongoing SOC treatment, and provide herein a pharmacogenomic and response-guided approach to treatment of these individuals, which is readily expanded to other infectious conditions or diseases. The present invention provides evidence that the response rates of C/T individuals can be substantially improved by using immunotherapy, including in interferon-naïve individuals and prior non-responders to interferon-based therapy.

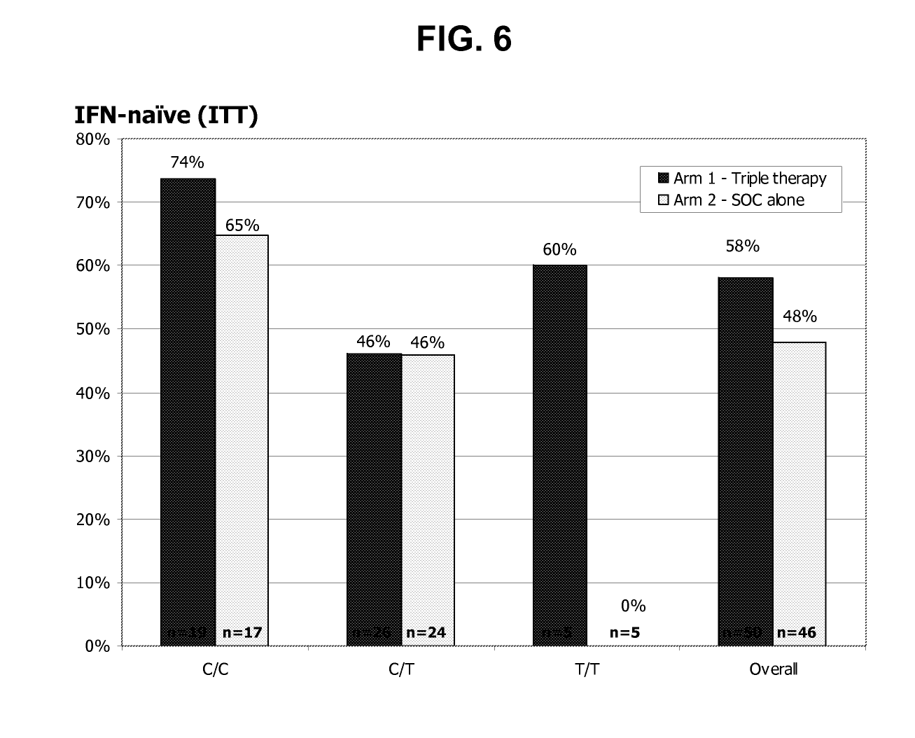

More particularly, although both triple therapy and SOC C/T individuals achieved the same rates of SVR in the study described herein (see FIG. 6), examination of the individual responses and response kinetics (see FIG. 6) reveals characteristics of the response when yeast-based immunotherapy is added (triple therapy) that can now be used to improve the response of C/Ts (which also applies generally to T/T patients, discussed in more detail below). Specifically, in both triple therapy and SOC alone, C/Ts had a later time course to complete response (viral negativity), with increased numbers of individuals reaching viral negativity after the first 12 weeks of treatment, i.e., after the significant early response timepoint (EVR) used to predict treatment success. However, the SOC treatment group lost C/T responders on therapy (i.e., between weeks 24-48 when drug was still being administered), whereas the triple therapy treatment group substantially maintained complete responses in C/Ts during this same period of time on therapy, achieving a better ETR for C/Ts on triple therapy (see FIG. 6). It was only after treatment ended at 48 weeks (ETR) that C/Ts in the triple therapy group experienced enough relapses to move the total percentage of complete responses at SVR to the same rate as C/Ts on SOC alone (notably, C/Ts in the SOC group also lost responders post-treatment). A review of C/T individuals who first achieved viral negativity during the 24-48 week period and who subsequently relapsed post-treatment provides additional insight into the response of these individuals to immunotherapy. Specifically, referring to FIG. 6, in the triple therapy group, it is generally observed that the later during the therapy period that an individual first achieves viral negativity on treatment, the sooner the individual appears to relapse post-treatment. These data indicate that C/Ts respond more slowly to therapy (either type) and while continuing on triple therapy (extrapolated to any SOC plus yeast-based immunotherapy), appear to maintain viral negativity, in contrast to SOC. However, if the C/T's achieve viral negativity late in the treatment period, many are not able to maintain viral negativity once therapy is removed, and the data as a whole indicates that if these individuals had remained on therapy longer, a better outcome may have resulted.

Accordingly, the present inventors propose herein that C/Ts receiving immunotherapy should be monitored during therapy to identify those individuals who are slow responders to the combination of immunotherapy and the SOC for a given infectious disease, as measured by failure to reach a particular milestone that is indicative or predictive of a positive response in that disease, whether it is a time-based endpoint or a clinical milestone. For example, patients for whom treatment with yeast-based immunotherapy combined with SOC should be extended include C/T patients chronically infected with HCV who first reach viral negativity after 12-24 weeks of SOC therapy that includes yeast-based immunotherapy, or after the time that is determined to be most predictive of when patients receiving SOC alone will progress to SVR. Similarly, for C/T patients chronically infected with HBV who do not achieve seroconversion even after achieving viral negativity, or who have remissions, extension or addition of yeast-based therapy to the regimen, or extension of yeast-based immunotherapy if it is already used in the regimen, is contemplated by the invention. Alternatively, C/T patients can initially be prescribed a longer term of treatment using a regimen that includes yeast-based immunotherapy. In one embodiment, C/T individuals continue to receive yeast-based immunotherapy/SOC regimen for an extended period of time, to allow the therapy a beyond the standard end of treatment (e.g., at 48 weeks for HCV interferon-naïve individuals or at 72 weeks for HCV non-responder individuals under current SOC regimens) or beyond a clinical milestone, such as seroconversion. By extending therapy using yeast-based immunotherapy, it is believed that a substantially larger percentage of C/T individuals will achieve complete response (e.g., complete response at SVR for HCV, seroconversion for HBV or seroconversion without remission for a period of several months, etc.). The data presented herein shows that single patients can be monitored for responsiveness under immunotherapy-based regimens and their therapy can be personalized by extending or modifying treatment based on genotype combined with first time to responsiveness, in order to optimize their chance of achieving a complete response to the therapy. For example, C/T patients receiving immunotherapy may be able to modify dosage, modify the combination of agents administered, and/or modify the total time of treatment without sacrificing therapeutic outcome and indeed, while improving therapeutic outcome as compared to C/T patients under SOC-only protocols.

Therefore, for individuals having an IL28B C/T genotype who achieve a positive response to therapy within the period predicted to be most successful for SOC or who achieve a clinical milestone that predicts successful treatment, based on the data provided herein, adding immunotherapy to SOC therapy is also expected to produce a meaningful advantage in such individuals, even while using the same therapy protocol as for SOC (e.g., without extending the therapy period). For C/T individuals who achieve a positive response to therapy after the period predicted to be most successful for SOC, or who do not achieve a clinical milestone associated with successful treatment after a given period of time, modifying the therapeutic protocol, such as by extending the period of administration of immunotherapy with SOC beyond the standard SOC protocol, is expected to be more effective at delivering such C/T patients to a sustainable complete response. The extended period can be adjusted depending on how late in the therapy period a particular patient achieves a successful response or achieves a clinical milestone. For example, in HCV, a patient achieving viral negativity at 24 weeks of therapy may receive therapy for a longer total period of time than a patient who achieved viral negativity at 20 weeks, particularly if the patient tolerates the therapy well. In addition, by using immunotherapy, other parameters can be modified, including, but not limited to adjusting dosage of the therapeutic agents and/or modifying the combination of agents used. The present invention has provided evidence that response-guided therapy based on genotype is an effective method for realizing the advantages of immunotherapy without being restrained by prior predictors of outcome based on SOC alone. Prior to the present invention, such a personalized approach, or response-guided approach, to therapy for HCV and other infectious disease based on the IL28B genotype was not available.

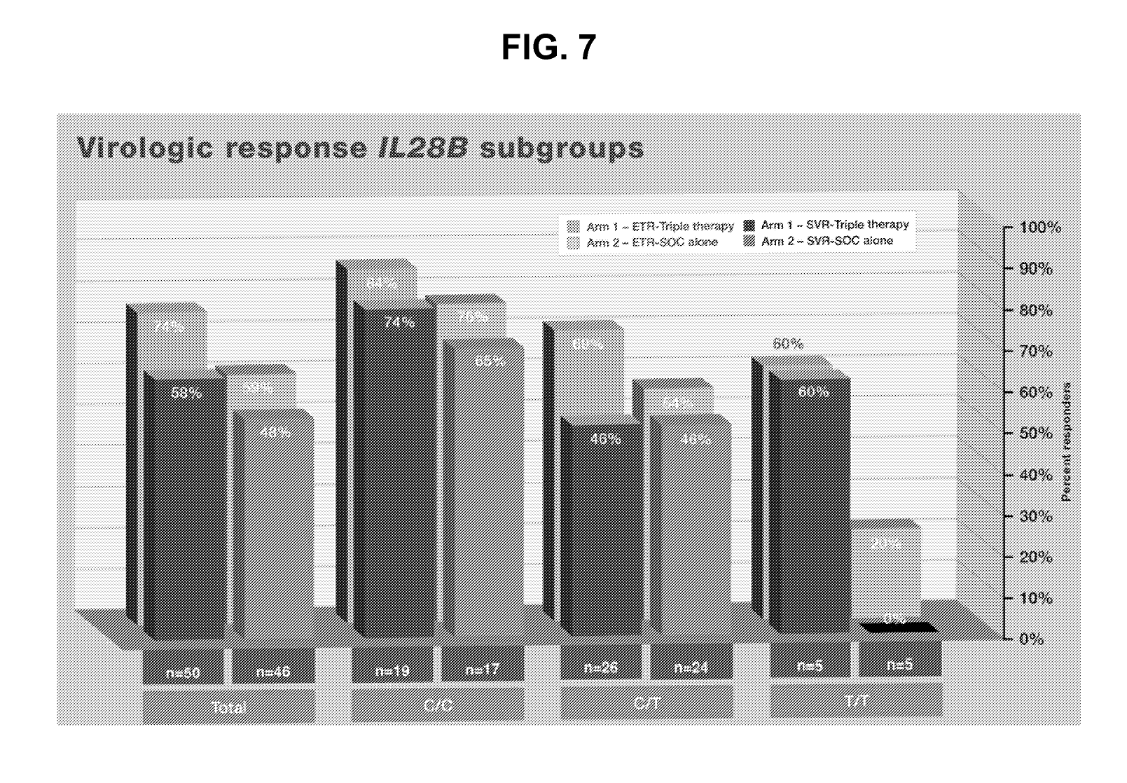

According to the present invention, “TIT” individuals, who are individuals having the T/T genotype at the IL28B locus (described in detail below), predicted to have a poor prognosis for responding to conventional treatment regimens for HCV (e.g., SOC, such as combinations of direct-acting agents, such as anti-viral drugs and/or interferons). For example, in chronic HCV infection, only approximately 26% of these individuals will achieve SVR in response to SOC therapy) (Ge et al., supra). The inventors have now discovered how T/T individuals respond to yeast-based immunotherapy in the context of chronic HCV infection and ongoing SOC treatment, and provide herein a pharmacogenomic and response-guided approach to treatment of these individuals, which is readily expanded to other infectious conditions or diseases. The present invention provides evidence that the response rates of T/T individuals can be significantly improved by using immunotherapy. More particularly, in the study described herein, both ETR and SVR rates in T/T patients were significantly greater for triple therapy compared to SOC or historical controls (see FIG. 7), with 60% of the T/T patients achieving ETR and maintaining negativity to SVR, as compared to 0% of the patients receiving SOC alone, demonstrating that immunotherapy has a substantial impact in this high risk patient group. More specifically, triple therapy delivered patients who reached viral negativity prior to 12 weeks or after 12 weeks (slow responders) to SVR, whereas SOC alone was not able to deliver any T/T patients to SVR in this study. In addition, all T/T patients in the triple therapy group reached viral negativity by 24 weeks. Compared to the low SVR rate of 26% reported historically for SOC alone (0% reported in this study), immunotherapy demonstrated that the outcomes of this subgroup of patients can be changed from poor to good. In addition, the results described herein indicate that within this T/T subgroup, as with the C/T genotype patients described above, some patients achieved negatively later in therapy, after the 12 week EVR endpoint that is used in chronic HCV treatment with SOC as a predictor for positive outcomes. Such patients, if treated for an extended period of time (e.g., longer than 48 weeks total for interferon-naïve individuals and longer than 72 weeks total for non-responder individuals), can be expected to have an improved likelihood of reaching SVR as compared to patients for whom the standard SOC protocol is utilized. Accordingly, the present inventors propose that T/Ts receiving immunotherapy should be monitored during therapy to identify those individuals who are slow responders to the combination of immunotherapy and the SOC for a given infectious disease, as measured by failure to reach a particular milestone that is indicative or predictive of a positive response in that disease, whether it is a time-based endpoint or a clinical milestone. For example, patients for whom treatment with yeast-based immunotherapy combined with SOC should be extended include T/T patients chronically infected with HCV who first reach viral negativity after 12-24 weeks of SOC therapy that includes yeast-based immunotherapy, or after the time that is determined to be most predictive of when patients receiving SOC alone will progress to SVR. Similarly, for T/T patients chronically infected with HBV who do not achieve seroconversion even after achieving viral negativity, or who have remissions, extension or addition of yeast-based therapy to the regimen, or extension of yeast-based immunotherapy if it is already used in the regimen, is contemplated by the invention. Alternatively, T/T patients can initially be prescribed a longer term of treatment using a regimen that includes yeast-based immunotherapy. T/T patients can be monitored for responsiveness under immunotherapy-based regimens, and their therapy can be personalized by extending or modifying treatment based on genotype combined with first time to responsiveness, in order to optimize their chance of achieving a complete response to the therapy. For example, T/T patients receiving immunotherapy may be able to modify dosage, modify the combination of agents administered, and/or modify the total time of treatment without sacrificing therapeutic outcome and indeed, while improving therapeutic outcome as compared to T/T patients under SOC-only protocols.

Therefore, for individuals having an IL28B T/T genotype who achieve a positive response to therapy within a period predicted to be most successful for SOC or who achieve a clinical milestone that predicts successful treatment, adding immunotherapy to SOC therapy is expected to produce a meaningful advantage in such individuals, even while using the same therapy protocol as for SOC (e.g., without extending the therapy period). However, based on the data provided herein showing that such patients respond more slowly to therapy than do other patients, combined with the poor genotype-based prognosis of such patients based on prior studies, the present invention envisions extending therapy for most or in some scenarios, all, of these patients in order to maximize their opportunity to achieve a sustainable response. The data provided herein indicate that T/T individuals, as a group, respond later to therapy than individuals of C/C genotype, for example. For T/T individuals, extending the period of administration of immunotherapy with SOC beyond the standard SOC protocol is also expected to be more effective at delivering such T/T patients to a sustainable complete response. As with C/T patients, the extended period can be adjusted depending on how late in the therapy period a particular patient achieves a successful response. In addition, by using immunotherapy, other treatment parameters can be modified, including, but not limited to adjusting dosage of the therapeutic agents and/or modifying the combination of agents used and/or modifying the frequency of administration of agents.

One embodiment of the invention relates to a method to treat an infectious disease in an individual using a therapeutic protocol that comprises (includes) the administration of an immunotherapeutic composition, including a yeast-based immunotherapeutic composition, to the individual. According to the present invention, an infectious disease is defined as any disease caused by infection by a pathogenic or infectious organism. The present invention excludes methods for the diagnosis, prevention or treatment of cancer, but rather is directed to the prevention or treatment of infection by a pathogenic or infectious agent and symptoms associated with the infection, but excludes the diagnosis, prevention, palliation, or treatment of cancer. In one aspect of any embodiment described herein, the infectious disease is a viral disease. In one embodiment of the invention, a method to treat viremia or chronic viremia is contemplated. As used herein, viremia refers to the presence of virus in the bloodstream. Preferably, viremia is reduced or eliminated. In one aspect of any embodiment described herein, the infectious disease is hepatitis virus infection. In one aspect of any embodiment described herein, the infectious disease is chronic hepatitis C virus (HCV) infection. In one aspect of any embodiment described herein, the infectious disease is hepatitis B virus (HBV) infection. Any reference generally herein to hepatitis, hepatitis virus, or hepatitis virus infection can refer to a virus including HCV or HBV, unless specified. In one aspect of the invention, the virus is human immunodeficiency virus (HIV) and the infection can include co-infection of HIV with another virus, such as a HCV and/or HBV. In one aspect of any embodiment described herein, the infectious disease is infection by a virus, a fungus, a bacterium, a helminth, a parasite, an ectoparasite, or a protozoa.

One element of one of the methods of the invention is that the IL28B genotype of the individual is known or is determined or learned prior to commencing the therapeutic protocol. According to the present invention, a practitioner of a method of treatment of the invention does not have to be the same individual or entity that performs the genotyping. The genotype of the individual can be previously determined by a diagnostic laboratory, for example, and the information can be provided to the practitioner by any suitable form of oral, written, electronic or other communication. In one embodiment, a kit for determining IL28B genotype (e.g., including reagents, probes, primers, and/or other agents useful for determining IL28B genotype or detecting IL28B polymorphism in a sample, such as a DNA sample) is provided along with therapeutic reagents (including a yeast-based immunotherapy reagent or composition) for treating the infectious disease or condition, and with instructions for using the same, based on the IL28B genotype of the subject. By knowing the IL28B genotype of the individual, the protocol that includes immunotherapy can be modified, if necessary, given the genotype of the individual and the predicted outcome of this individual to therapy based on the genotype.

The methods of the invention generally include treating individuals differently depending on whether the individual has an IL28B genotype of C/C, C/T, or T/T, and further based on the responsiveness of the individual to the therapeutic protocol, all with the goal of optimizing the response of the individual to the therapeutic protocol (i.e., pharmacogenomic guided response). Ideally, the individual will have a greater likelihood of achieving a complete response within the given infectious disease, where “complete response” typically means achieving negativity or significant reduction in pathogen burden with respect to detection of the infectious agent (as determined by the standard in the art for the given infection), achieving seroconversion for a particular antigen(s), achieving production or elimination of a particular biomarker, and/or achieving substantial reduction or complete elimination of symptoms that are directly associated with the infection. It will be appreciated that these measures are different for different infectious diseases, and such measures are provided herein for HCV and HBV, for example.

In general, individuals with a C/C phenotype will be administered the therapeutic protocol comprising yeast-based immunotherapy in accordance with the parameters defined for either a standard of care protocol in the absence of immunotherapy, or in accordance with the parameters defined to achieve clinically relevant complete response using the protocol comprising immunotherapy in all individuals (i.e., without regard to IL28B genotype), or in accordance with parameters previously defined to achieve clinically relevant complete response using the protocol comprising immunotherapy in C/C individuals. If desired, the C/C patient can be monitored for response to treatment and in some cases, dosage may be adjusted, the combination of treatment agents may be modified, and/or the treatment protocol may be shortened or extended. In one aspect, individuals with a C/C genotype are administered yeast-based immunotherapy in combination with additional agents used to treat the infectious disease (e.g., agents that act directly on the pathogen, cytokines such as interferon, etc.), and the protocol is modified to reduce the duration of administration, the dosage, and/or the frequency of administration of one or more of the additional agents. In one aspect, one or more agents that would used in a standard of care protocol in the absence of the immunotherapy of the invention is eliminated (e.g., interferon is eliminated). In one aspect, when yeast-based immunotherapy is added to the standard of care protocol, the total time of treatment using all agents is reduced.

In one aspect, individuals with a C/T genotype or a T/T genotype are initially administered the therapeutic protocol comprising immunotherapy in accordance with the parameters defined for either a standard of care protocol in the absence of immunotherapy, or in accordance with the parameters defined to achieve clinically relevant complete response using the protocol comprising immunotherapy in all individuals (i.e., without regard to IL28B genotype), or in accordance with parameters previously defined to achieve clinically relevant complete response using the protocol comprising immunotherapy in C/C individuals. The therapeutic protocol is then modified, such as by extension of the period of time the protocol is administered, for C/T or T/T individuals who first respond to the therapeutic protocol later than the average time period for response in all individuals or for individuals having an IL28B genotype of C/C, or by a defined period of treatment that commences after a particular clinical milestone is reached. For example, in the latter case, in a patient chronically infected with HBV, yeast-based immunotherapy is administered in combination with a standard of care therapy (e.g., an anti-viral drug) until a clinical endpoint of viral negativity or seroconversion is reached. At this point, the patient is treated for an additional number of months (e.g., 6-12 months) with the combination therapy. This type of response guided therapy allows the individual to respond outside of a rigorous timeline, and then extends therapy for this individual for an additional period of time to enhance the success of the therapy. In some cases, dosage of the therapeutic agents may be adjusted and/or the combination of treatment agents may be modified according to the response of the IL28B C/T or T/T individual. In one embodiment, the individual is monitored for responsiveness to the therapeutic protocol at selected time intervals and, if the individual is a slow responder to the therapeutic protocol, the individual is treated for a longer period of time than an individual with an IL28B C/C genotype or for a longer period of time than the SOC or new standard protocol specifies (and/or the agents and/or doses of agents used to treat the individual are altered). With respect to T/T individuals, in one embodiment, the individual is initially treated for a longer period of time than an individual with an IL28B C/C genotype, or for a longer period of time than the SOC or new standard protocol specifies (and/or the combination of agents and/or doses of agents used to treat the individual are altered). Such individuals can be monitored for responsiveness and the therapeutic protocol modified as needed, e.g., by further extending the time of treatment, and/or by altering the agents and/or doses of agents used to treat the individual.

One embodiment of the invention relates to a method to treat hepatitis virus infection in an individual. The method comprises treating the individual with a therapeutic protocol comprising administration of an immunotherapeutic composition, such as a yeast-based immunotherapeutic composition, comprising at least one hepatitis virus antigen or immunogenic domain thereof. The IL28B genotype of the individual is determined prior to administering the protocol, and therapeutic protocol is modified according to the IL28B genotype of the patient as described in detail herein. For example, in IL28B C/C individuals, a dose sparing approach may be utilized, where the duration, dosage and or frequency of one or more agents in the therapeutic regimen is reduced or eliminated. In IL28B C/T or T/T individuals, for example, the period of time of administration of the therapeutic protocol is extended for individuals who respond more slowly than a designated time-based endpoint, or the agents are administered for a designated time after a clinical milestone is reached, and/or the combination of agents and/or doses of agents used to treat the individual are altered.

According to the present invention, with respect to any embodiment of the invention described herein, a “clinical milestone” is a measurable or detectable clinical event during the treatment of the individual for an infectious disease that is used or is useful for determining the status of the individual during treatment, and/or for predicting therapeutic outcome of the individual to treatment, or for evaluating the outcome of the therapeutic treatment (e.g., for milestones that are clinical endpoints). For example, as described in detail elsewhere herein, clinical milestones for chronic HCV infection include viral negativity and normalized ALT, as well as the viral status of the individual at time points including RVR, EVR, ETR and SVR. Clinical milestones for HBV include viral negativity, seroconversion, and remission-free disease.

According to the present invention, with respect to any embodiment of the invention described herein, to “extend the period of time” of administration of a therapeutic protocol, to administer an agent for “longer” or a “longer period of time”, or any alternative phrasing of these phrases, means that the protocol, as compared to the protocol that was initially administered (which may include the therapeutic protocol comprising immunotherapy administered in accordance with the parameters defined for either a standard of care protocol in the absence of immunotherapy, or in accordance with the parameters defined to achieve clinically relevant complete response using the protocol comprising immunotherapy in all individuals (i.e., without regard to IL28B genotype), or in accordance with parameters previously defined to achieve clinically relevant complete response using the protocol comprising immunotherapy in C/C individuals), is extended for a suitable period of time to provide a longer period of time for such individuals to respond to the therapeutic protocol. Such an extended period of time will depend on the infectious disease to be treated, and can be an additional 1, 2, 3, 4, 5, 6 or 7 days, or an additional 1, 2, 3, 4, 5, 6, 7, 8, 9, 10, 11, 12, 13, 14, 15, 16, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more weeks, or an additional 1, 2, 3, 4, 5, 6, 7, 8, 9, 10, 11, 12, or more months of therapy. In some aspects, in addition to extending the period of time of treatment, the agents used can be modified and/or the dose of agents can be modified. In one aspect, one or more agents are administered for at least several weeks longer than the reference period of time or beyond achievement of a clinical milestone. In one aspect, one or more agents are administered for at least 4 to 48 additional weeks, which includes any number of weeks between 4 and 48 (e.g., 4, 5, 6, 7, 8 . . . 12 . . . 24 . . . 36 . . . 48). In one aspect, one or more agents are administered for at least 1, 2, 3, 4, 5, 6, 7, 8, 9, 10, 11 or 12 additional months beyond the reference time point or clinical milestone.

In one embodiment, the therapeutic regimen that includes yeast-based immunotherapy is continued for a designated period of time after a clinical milestone is reached. In this embodiment, the time of treatment before achieving the clinical milestone is not defined but rather, the time of additional treatment after the milestone is reached is defined. For example, in HBV treatment, patients are typically treated based on clinical milestones, such as seroconversion and loss of one or more HBV antigens, where treatment can last for months or years. Even in HCV treatment, instead of using current standard of care time-based endpoints for treatment decisions, the use of clinical milestones can be incorporated into the therapy, where, for example, patients are treated until viral negativity is first achieved, and then treatment proceeds for a standard period following this milestone (e.g., an additional 12, 24, 36, 48 months, or longer).

To adjust, modify or alter the dosage of a therapeutic agent means to increase or decrease the amount of a given therapeutic agent in order to change the amount of agent delivered to an individual in a single administration. Dosage may be adjusted for one agent in a combination therapy (e.g., dose of a more toxic agent may be reduced while doses of other agents remain unchanged) or for more than one agent in a combination therapy. In one aspect of the invention, the dosage is modified so that the dosage of an agent is reduced as compared to a dosage that is or was previously established as effective for the treatment of the infectious disease in the absence of including yeast-based immunotherapy in the protocol or regimen.

To adjust, modify or alter the duration of administration of a therapeutic agent means to increase or decrease the amount of time that a given therapeutic agent is administered (either total or per cycle) in order to change the total amount of agent delivered to an individual or to change the time for which the patient receives the given agent. The duration of administration may be adjusted for one agent in a combination therapy (e.g., a more toxic agent may be eliminated after a given period of time, while doses of other agents remain unchanged) or for more than one agent in a combination therapy. In one aspect of the invention, the duration of administration is modified so that the total period of time that an agent is administered is lengthened as compared to the total period of time that is or was previously established as effective for the treatment of the infectious disease in the absence of including yeast-based immunotherapy in the regimen. In one aspect of the invention, it may be desirable to shorten the duration of administration of an agent so that the total period of time that an agent is administered is reduced as compared to the total period of time that is or was previously established as effective for the treatment of the infectious disease in the absence of including yeast-based immunotherapy in the regimen.

To adjust, modify or alter the frequency of administration of a therapeutic agent means to increase or decrease the time between doses of a given therapeutic agent. The frequency of administration may be adjusted for one agent in a combination therapy (e.g., frequency of administration of a more toxic agent may be reduced while the frequency of administration of other agents remain unchanged) or for more than one agent in a combination therapy. In one aspect of the invention, the frequency of administration is modified so that the agent is administered more or less often than the frequency of administration for the agent that is or was previously established as effective for the treatment of the infectious disease in the absence of including yeast-based immunotherapy in the regimen.

Reference herein to a dosage of an agent or a protocol for administration as being “established as effective” means that a given dosage or dosage range, frequency of administration, route of administration, and/or duration of administration for the agent has been previously established to be effective for a given purpose, typically via regulatory approval for the use of an agent with respect to a given disease or condition. For example, a dosage of pegylated interferon-α that is established as effective for the treatment of HCV in the absence of a yeast-based immunotherapeutic is the recommended dose of interferon when used in combination with ribavirin for chronic hepatitis C, which is 180 μg (1.0 mL vial or 0.5 mL prefilled syringe) once weekly (e.g., for PEGASYS®, Roche). Accordingly, in a protocol that included yeast-based immunotherapy, in one aspect of the invention, one could reduce that dosage for interferon (or increase the dosage, if deemed necessary), or one could modify the frequency of administration to a frequency that is less often than once weekly (or more often, if deemed necessary), or one could administer the interferon for a total period of time that is shorter than or longer than the standard protocol or therapeutic regimen (e.g., a standard protocol being 48 weeks for interferon-naïve patients chronically infected with genotype 1 HCV).

To adjust, modify or alter the combination of agents administered, means to change at least one agent in a combination of agents by eliminating that agent or substituting a different agent for that agent, or to add a new agent to the existing combination of agents. For example, in a combination of immunotherapy, interferon-α and ribavirin, the ribavirin might be eliminated or replaced with a different anti-viral, or another anti-viral or host enzyme inhibitor might be added to the combination (perhaps in conjunction with altering the doses of one or more agents). Alternatively, the interferon component may be eliminated. As another example, yeast-based immunotherapy may be combined with ribavirin, interferon, and with a viral protease inhibitor, where the interferon, for example, may be eliminated from the combination. Virtually any combination of yeast-based immunotherapy with other therapeutic agents for the treatment of infectious disease is envisioned.

Another embodiment of the invention relates to a method to treat hepatitis virus infection in an individual, including, but not limited to, hepatitis B virus (HBV) infection or hepatitis C virus (HCV) infection. The method includes treating the individual with a therapeutic protocol comprising administration of: (a) a yeast-based immunotherapeutic composition comprising at least one hepatitis virus antigen or immunogenic domain thereof, wherein the immunotherapeutic composition elicits a T cell-mediated immune response against one or more hepatitis virus antigens; and (b) one or more agents selected from: an interferon, an anti-viral compound, a host enzyme inhibitor, and/or an immunotherapeutic composition other than the immunotherapeutic composition of (a).

In one aspect of this embodiment, the therapeutic protocol is modified for individuals having an IL28B genotype of C/C by reducing the duration, dosage, and/or frequency of administration one or more of the agents of (b), or by shortening the total time of administration of the therapeutic protocol, as compared to the time of administration of the therapeutic protocol in the absence of a yeast-based immunotherapeutic.

In one aspect of this embodiment, the therapeutic protocol is modified for individuals having an IL28B genotype of C/T by monitoring the responsiveness of these individuals to the protocol and: (1) extending the period of time of administration of the protocol for those individuals who are slow responders to the protocol, (2) providing a given period of time after a clinical endpoint beyond which therapy will be extended, and/or (3) modifying duration, dosage, and/or frequency of administration one or more of the agents of (b).

In one aspect of this embodiment, the therapeutic protocol is modified for individuals having an IL28B genotype of T/T by monitoring the responsiveness of these individuals to the protocol and: (1) extending the period of time of administration of the protocol for those individuals who are slow responders to the protocol, (2) providing a given period of time after a clinical endpoint beyond which therapy will be extended, and/or (3) modifying duration, dosage, and/or frequency of administration one or more of the agents of (b). In one aspect, the protocol is automatically modified for individuals with a TT genotype by extending the total time of treatment, by providing a given period of time after a clinical endpoint beyond which therapy will be extended, and/or (3) modifying duration, dosage, and/or frequency of administration one or more of the agents of (b).

Another embodiment of the invention is a method to treat chronic hepatitis C virus (HCV) infection, and/or to prevent, ameliorate or treat at least one symptom of chronic HCV infection, in an individual comprising administering to an individual: (a) an immunotherapeutic composition, such as a yeast-based immunotherapeutic composition, comprising at least one HCV antigen or immunogenic domain thereof, wherein the immunotherapeutic composition elicits a T cell-mediated immune response against one or more HCV antigens; and (b) one or both of at least one interferon and at least one anti-viral compound. In one aspect, the immunotherapeutic composition and the interferon and/or anti-viral compound are administered concurrently over a period of 48 weeks to interferon-naïve individuals having an IL28B genotype of C/C or C/T, and over a period of 72 weeks to non-responder individuals having an IL28B genotype of C/C or C/T, except that, if the individual having an IL28B genotype of C/T does not reach viral negativity within the first 12 weeks of the period, then the immunotherapeutic composition and the interferon and/or anti-viral compound are administered concurrently for a period greater than 48 weeks for interferon-naïve individuals and for a period greater than 72 weeks for non-responder individuals. In another embodiment, if an individual having a C/C genotype does not reach viral negativity within the first 12 weeks of the period, then the immunotherapeutic composition and the interferon and/or anti-viral compound are administered concurrently for a period greater than 48 weeks for interferon-naïve individuals and for a period greater than 72 weeks for non-responder individuals.

In one embodiment, the immunotherapeutic composition and the interferon and/or anti-viral compound are administered concurrently over a period of less than 48 weeks to individuals having an IL28B genotype of C/C. In one embodiment, the dosage, the duration of administration, or the frequency of administration of either the anti-viral(s) or the interferon is reduced for individuals having an IL28B genotype of C/C, as compared to the dosage given to the other genotypes, or as compared to the dosage typically provided in the absence of inclusion of the yeast-based immunotherapy. In one embodiment, interferon is eliminated from the protocol for C/C individuals.

Additionally, in one embodiment, the immunotherapeutic composition and the interferon and/or anti-viral compound are administered concurrently over a period of 48-72 weeks for all individuals (interferon-naïve and non-responder) having an IL28B genotype of T/T, except that, if the individual does not reach viral negativity within the first 12-24 weeks, then the immunotherapeutic composition and the interferon and/or anti-viral compound are administered concurrently for a period greater than the 48-72 weeks. In another embodiment, the immunotherapeutic composition and the interferon and/or anti-viral compound are administered concurrently over a period of 48 weeks for all interferon-naïve individuals having an IL28B genotype of T/T and for a period of 72 weeks for all non-responder individuals having an IL28B genotype of T/T, except that, if the individual does not reach viral negativity within the first 12-24 weeks, then the immunotherapeutic composition and the interferon and/or anti-viral compound are administered concurrently for a period greater than 48 weeks for interferon-naïve individuals and for a period greater than 72 weeks for non-responder individuals. In one embodiment, the dosage, the duration of administration, or the frequency of administration of either the anti-viral(s) or the interferon is reduced. In one embodiment, interferon is eliminated from the protocol.

Another embodiment of the invention is a method to treat chronic hepatitis C virus (HCV) infection, and/or to prevent, ameliorate or treat at least one symptom of chronic HCV infection, in an individual comprising administering to an individual: (a) an immunotherapeutic composition, such as a yeast-based immunotherapeutic composition, comprising at least one HCV antigen or immunogenic domain thereof, wherein the immunotherapeutic composition elicits a T cell-mediated immune response against one or more HCV antigens; and (b) one or both of at least one interferon and at least one anti-viral compound. In one aspect, the immunotherapeutic composition and the interferon and/or anti-viral compound are administered to a patient having an IL28B genotype of C/T or T/T until the patient first reaches viral negativity, followed by an additional 24 weeks, 36 weeks, 48 weeks, 60 weeks or more of treatment using the combination therapy. In one aspect, the dosage, the duration of administration, or the frequency of administration of either the anti-viral(s) or the interferon is reduced. In one embodiment, interferon is eliminated from the protocol.

Another method of the invention relates to a method to treat chronic hepatitis C virus (HCV) infection, and/or to prevent, ameliorate or treat at least one symptom of chronic HCV infection, in an individual comprising administering to an individual: (a) an immunotherapeutic composition, such as a yeast-based immunotherapeutic composition, comprising at least one HCV antigen or immunogenic domain thereof, wherein the immunotherapeutic composition elicits a T cell-mediated immune response against one or more HCV antigens; (b) pegylated interferon-α; and (c) ribavirin. In one aspect of this embodiment, the immunotherapeutic composition, the pegylated interferon-α, and the ribavirin are administered concurrently over a period of 48 weeks to interferon-naïve individuals having an IL28B genotype of C/C, and over a period of 72 weeks to non-responder individuals having an IL28B genotype of C/C. In one embodiment, the immunotherapeutic composition, the pegylated interferon-α, and the ribavirin are administered concurrently over a period of less than 48 weeks to individuals having an IL28B genotype of C/C. In one embodiment, the dosage, the duration of administration, or the frequency of administration of either the ribavirin or the pegylated interferon-α is reduced for individuals having an IL28B genotype of C/C, as compared to the dosage given to the other genotypes, or as compared to the dosage typically provided in the absence of inclusion of the yeast-based immunotherapy. In one embodiment, interferon is eliminated from the protocol for C/C individuals.

In one aspect of this embodiment, the immunotherapeutic composition, the pegylated interferon-α, and the ribavirin are administered concurrently over a period of 48 weeks to interferon-naïve individuals having an IL28B genotype of C/T, and over a period of 72 weeks to non-responder individuals having an IL28B genotype of C/T, except that, if the individual having an IL28B genotype of C/T does not reach viral negativity within the first 12 weeks of the period, then the immunotherapeutic composition, the pegylated interferon-α, and the ribavirin are administered for a period greater than 48 weeks for interferon-naïve individuals and for a period greater than 72 weeks for non-responder individuals.

In one aspect, the immunotherapeutic composition, the pegylated interferon-α and the ribavirin are administered concurrently over a period of 72 weeks for all individuals (interferon-naïve and non-responder) having an IL28B genotype of T/T, except that, if the individual does not reach viral negativity within the first 12-24 weeks, then the immunotherapeutic composition and the pegylated interferon-α and/or anti-viral compound are administered concurrently for a period greater than 72 weeks. In another aspect, the immunotherapeutic composition, the pegylated interferon-α and the ribavirin are administered concurrently over a period of 48 weeks for all interferon-naïve individuals having an IL28B genotype of T/T and for a period of 72 weeks for all non-responder individuals having an IL28B genotype of T/T, except that, if the individual does not reach viral negativity within the first 12-24 weeks, then the immunotherapeutic composition, the pegylated interferon-α and the ribavirin are administered concurrently for a period greater than 48 weeks for interferon-naïve individuals and for a period greater than 72 weeks for non-responder individuals.

Another method of the invention relates to a method to treat chronic hepatitis C virus (HCV) infection, and/or to prevent, ameliorate or treat at least one symptom of chronic HCV infection, in an individual comprising administering to an individual: (a) an immunotherapeutic composition, such as a yeast-based immunotherapeutic composition, comprising at least one HCV antigen or immunogenic domain thereof, wherein the immunotherapeutic composition elicits a T cell-mediated immune response against one or more HCV antigens; (b) pegylated interferon-α; (c) ribavirin, and (d) an HCV protease inhibitor. In one aspect of this embodiment, the immunotherapeutic composition, the pegylated interferon-α, and the ribavirin are administered concurrently over a period of 24-48 weeks to individuals having an IL28B genotype of C/C. In one embodiment, the immunotherapeutic composition, the pegylated interferon-α, and the ribavirin are administered concurrently over a period of less than 24-48 weeks to individuals having an IL28B genotype of C/C. In one embodiment, the dosage, the duration of administration, or the frequency of administration of the ribavirin, the protease inhibitor, or the pegylated interferon-α is reduced for individuals having an IL28B genotype of C/C, as compared to the dosage given to the other genotypes, or as compared to the dosage typically provided in the absence of inclusion of the yeast-based immunotherapy. In one embodiment, interferon is eliminated from the protocol for C/C individuals. In one aspect, the immunotherapeutic composition, the pegylated interferon-α, the ribavirin and the protease inhibitor are administered concurrently over a period of 24-48 weeks for all individuals (interferon-naïve and non-responder) having an IL28B genotype of C/T or T/T, except that, if the individual does not reach viral negativity within the first 12 weeks, then the immunotherapeutic composition and the pegylated interferon-α, ribavirin and protease inhibitor are administered concurrently for a period greater than 24-48 weeks.

Another embodiment of the invention relates to a method to treat chronic hepatitis B virus (HBV) infection, and/or to prevent, ameliorate or treat at least one symptom of chronic HBV infection, in an individual. HBV infection is typically diagnosed in an individual by detection of HBsAg (hepatitis B virus surface antigen) and/or HBeAg (e-antigen) in the blood of the infected individual. In addition, chronic HBV infection can be diagnosed by identifying HBV DNA (>2000 IU/ml) and/or elevated ALT levels. Recovery from the viral infection (complete response, the endpoint for treatment) is determined by HBeAg/HBsAg seroconversion, which is loss of HBeAg and HBsAg and the development of antibodies against the hepatitis B surface antigen (anti-HBs) and/or antibodies against HBeAg. Seroconversion can take years to develop in a chronically infected patient under current standard of care treatment (i.e., anti-viral drugs or interferon). Patients can also be monitored for loss or marked reduction of viral DNA (below detectable levels by PCR or <2000 IU/ml), normalization of serum alanine aminotransferase (ALT) levels, and improvement in liver inflammation and fibrosis. “ALT” is a well-validated measure of hepatic injury and serves as a surrogate for hepatic inflammation. In prior large hepatitis trials, reductions and/or normalization of ALT levels (ALT normalization) have been shown to correlate with improved liver function and reduced liver fibrosis as determined by serial biopsy.

Individuals are usually treated for HBV using approved interferon or anti-virals when they have elevated ALT levels together with elevated HBV DNA (>20000 IU/ml) and/or detectable HBeAg. In chronic HBV infection, SOC may be one of several different approved therapeutic protocols, and include, but may not be limited to, interferon therapy or anti-viral therapy. Current SOC for treatment of individuals with chronic HBV infection includes interferon or anti-viral compounds. Currently approved anti-viral drugs for HBV infection include lamivudine (EPIVIR®), adefovir (HEPSERA®), tenofovir (VIREAD®), telbivudine (TYZEKA®) and entecavir (BARACLUDE®).

The method of treatment of chronic hepatitis B virus infection comprises administering to an individual: (a) an immunotherapeutic composition, such as a yeast-based immunotherapeutic composition, comprising at least one HBV antigen or immunogenic domain thereof, wherein the immunotherapeutic composition elicits a T cell-mediated immune response against one or more HBV antigens; (b) one or more agents selected from interferon, lamivudine, adefovir, tenofovir, telbivudine, and entecavir. The immunotherapeutic composition and the one or more agents are administered concurrently to individuals having an IL28B genotype of C/C over a period of time established as effective for the agents of (b) or until the individual reaches HBeAg or HBsAg seroconversion. Following this clinical endpoint, the individual is treated for another 1, 2, 3, 4, 5, 6, 7, 8, 9, 10, 11 or 12 months with the combination therapy. In one embodiment, the dosage, the duration of administration, or the frequency of administration of the anti-viral or interferon is reduced for individuals having an IL28B genotype of C/C, as compared to the dosage given to the other genotypes, or as compared to the dosage typically provided in the absence of inclusion of the yeast-based immunotherapy. The immunotherapeutic composition and the one or more agents are administered concurrently to individuals having an IL28B genotype of C/T or T/T over a period of time established as effective for the agents of (b), or until the individual reaches HBeAg or HBsAg seroconversion. Following this clinical endpoint, the individual is treated for another 1, 2, 3, 4, 5, 6, 7, 8, 9, 10, 11, 12, 13, 14, 15, 16, 17, 18, 19, 20, 21, 22, 23, or 24 months with the combination therapy and/or the agents, combination of agents, and/or doses of agents used to treat the individual are modified. In one aspect, C/T and/or T/T individuals are treated for a period after seroconversion that is between 1 and 12 months longer than C/C individuals are treated after seroconversion. In one embodiment, each individual, regardless of IL28B genotype, is treated until the individual reaches HBeAg or HBsAg seroconversion, followed by an additional 1-24 months of treatment using the same regimen. In one embodiment, a significantly higher number of T/T individuals receiving therapy that includes yeast-based immunotherapy reaches seroconversion and remission-free status than T/T individuals receiving therapy that does not include yeast-based immunotherapy.

Various aspects and definitions related to any of the embodiments of the invention are described below.

### IL28B Genotype and Methods of Identifying the Genotype of an Individual

According to the present invention, reference to “IL28B genotype” or any derivation or similar usage of this phrase, refers to a genotype associated with the identification of a polymorphism that actually resides approximately 3 kilobases (kb) upstream of the IL28B gene, which encodes interferon-λ3, on chromosome 19. Therefore, while it is convenient to refer to the polymorphism as the “IL28B genotype”, the actual gene or genes impacted by the polymorphism is not known, although it appears that the impact is related to the immune response, and the fact that IL28B encodes interferon-λ3, a type III interferon, is interesting in this regard. Accordingly, the polymorphism may have an impact on the IL28B gene and/or it may impact a different gene or genes. The polymorphism, designated rs12979860, was initially characterized by Ge et al. in 2009 (Nature 461, 399-401) as being strongly associated with the outcome of SOC therapy for HCV (interferon/ribavirin therapy), which was soon confirmed by Tanaka et al. (2009, Nature Genetics 41:1105) and Suppiah et al. (2009, Nature Genetics 41:1100). Also in 2009, Thomas et al. (Nature 461, 798-801) showed that this polymorphism was also associated with the spontaneous clearance of HCV by individuals with acute infection, and was stated to be the strongest and most significant genetic effect associated with natural clearance of HCV to date. In addition to the rs12979860 polymorphism, several other closely correlated polymorphisms have been identified and associated with outcomes in spontaneous clearance of acute HCV infection and/or response to interferon-based therapy/SOC (e.g., rs28416813, rs8103142, rs8099917, rs12980275, rs7248668, rs11881222, or rs8105790, see Ge et al., supra, Suppiah et al., supra, Tanaka et al., supra, Rausch et al., Gastroenterology, 2010, 138:1338-45, and McCarthy et al., Gastroenterology, 2010, 138:2307-14). Since at least some of these polymorphisms are not currently separable from the effects of the rs12979860 locus, in one embodiment of the invention, any one or more of these other loci may be used to predict outcome to standard or interferon-based therapy, in addition to or in place of the rs12979860 locus.

Individuals fall into one of three genotypes at the rs12979860 locus: C/C (homozygous for the C allele), C/T (heterozygous for C and T alleles), or T/T (homozygous for the T allele). C/C individuals have the greatest likelihood of achieving SVR in response to SOC therapy, as shown in the table below (Table generated using data provided in Ge et al., 2009, supra), where as SVR rates in C/T are much poorer, and SVR rates in T/T are quite poor.

| | | | % of |
|---|---|---|---|
| | | SVR | population |
| Genotype | Prognosis | rate | with genotype |
| C/C | Good | 78% | 34% |
| C/T | Moderate | 37% | 49% |
| T/T | Poor | 26% | 16% |

Ge et al. and others showed that the polymorphism was strongly associated with SVR after SOC therapy in all patient groups, but the polymorphism also appears to explain previously observed differences in responses by different racial groups (i.e., much of the difference in response to SOC previously observed between individuals of European ancestry versus those of African-American ancestry can be accounted for by the frequency of the C allele in each population).

Ge et al., supra, also identified two other variants that were so closely correlated with the IL28B rs12979860 genotype, that tests for independence among the variants were not able to distinguish which, if any, of the polymorphisms were actually causally responsible for the observed phenotype. These polymorphisms, denoted rs28416813 (a G>C transition 37 bp upstream of the translation initiation codon for IL28B gene) and rs8103142 (a non-synonymous coding single nucleotide polymorphism encoding the amino acid substitution Lys70Arg in IFN-λ3), as well as any other polymorphisms that are highly correlated with the IL28B genotype described herein, are also encompassed by the present invention for use individually or in any combination in order to modify cancer therapy including immunotherapy for the treatment of an individual. Another polymorphism in the IL28B region that has been correlated with treatment responses in HCV is rs8099917, which is located in the intergenic region between IL28A and IL28B.

Any of the polymorphisms described herein, including the polymorphism at the rs12979860 locus, can be identified using any suitable genotyping method known in the art. Such methods are described, for example, in Ge et al., in Suppiah et al., in Tanaka et al., and in Thomas et al. Example 2 describes the method utilized in the present invention, which combines PCR with bi-directional sequencing. Other methods could include, but are not limited to, hybridization methods, primer extension methods, single strand conformation polymorphism methods, pyrosequencing methods, high resolution melting methods, and sequencing methods.

### Background and Definitions Related to Chronic HCV Infection

The current Standard Of Care (SOC) for the treatment of chronic hepatitis C is pegylated interferon-α plus ribavirin combination therapy, where the interferon is typically administered by subcutaneous injection once weekly for 24 weeks (HCV genotypes 2 and 3) or 48 weeks (HCV genotypes 1 and 4), with daily doses of ribavirin. While interferon/ribavirin therapy is relatively efficacious in patients suffering from genotype 2 or 3 HCV infection (˜85% of patients reach Sustained Virologic Response (SVR)), about 50% of patients infected with genotype 1 HCV do not reach SVR. Moreover, the current SOC is poorly tolerated—interferons are pro-inflammatory cytokines that are known to cause side effects, including flu-like symptoms and depression, and ribavirin induces hemolytic anemia in 20-30% of patients. When used together as Standard of Care (SOC), adverse events reported include flu-like symptoms (e.g., fever, headache, chills), gastrointestinal issues (e.g., nausea, anorexia, diarrhea), neuropsychiatric disorders (e.g., depression), skin disorders, and hematological disorders. These side effects often lead to patient non-compliance or discontinuation of treatment, and require erythropoietin rescue and/or dose reductions in 10-20% of patients.

The behavior of the serum HCV RNA levels in chronic HCV has been predicted in various settings using a 3 compartment model of viral kinetics, which includes uninfected liver cells, infected liver cells, and free virus in the serum. Viral levels in the peripheral blood early during the course of interferon (IFN) therapy have served as an early predictor of response to therapy due to the fact that they can be measured easily and have been correlated to other more meaningful endpoints in the setting of long-term IFN treatment, such as Sustained Virologic Response (SVR, defined as negative peripheral viral levels for at least 6 months after the completion of IFN-based therapy). Viral clearance in the setting of interferon therapy is bi-phasic; a rapid early phase of peripheral viral load reduction which occurs in the first week(s) (phase 1), followed by the rate limiting, gradual second phase of peripheral viral load reduction which occurs over many months (phase 2) (Layden-Almer et al., J Viral Hep 2006; 13:499-504; Herrmann and Zeuzem S. Eur J Gastroenterol Hepatol 2006; 18:339-342). While phase 1 kinetics reflect the efficiency of inhibition of viral replication (driven by rapid peripheral viral clearance), phase 2 kinetics represent direct clearance of infected liver cells. Clearance of infected hepatocytes is the rate limiting step in achieving complete eradication of hepatic infection and SVR.

While the ultimate goal of therapy is SVR, there are several early prognostic endpoints that serve as markers to guide patient treatment as summarized below.

| Endpoint | Definition | Predictive Value |
|---|---|---|
| Rapid Virologic | Viral negativity at | 90-100% of RVRs (prior |
| Response (RVR) | week 4 of IFN therapy | treatment naïve subjects) |
| | | will reach SVR^{1,2,3} |
| Early Virologic | >2 log10 reduction in | <3% of non-EVRs will |
| Response (EVR) | viral load at week 12 | reach SVR⁴; 60-75% of |
| | of interferon therapy | EVRs reach SVR^{3,5,6,7} |
| Complete EVR | Viral negativity at | ~90% of cEVRs will |
| (cEVR) | week 12 of IFN therapy | reach SVR⁵ |
| End of Treatment | Viral negativity at | ~80% of ETRs will |
| Response (ETR) | 48 weeks (genotype 1) | achieve SVR⁸ |
| Sustained Virologic | Viral negativity at | ~98% of subjects |
| Response (SVR or | 6 months post-ETR | achieving SVR24 will |
| SVR24) | | remain virus free 5 years |
| | | out⁹ |

¹Yu et al, RVR and treatment duration in CHC: a randomized trial; Hepatology 2008
²Jensen et al, Early ID of HCV G1 patients responding to 24 wks of treatment; Hepatology 2006
³Schiffman M L (2007) “New Management Strategies for HCV Nonresponders and Relapsers”
⁴Pegasys prescribing information 2008; Roche
⁵Brandao et al, 24 vs 48 weeks of Pegasys (Riba) in (Geno 1, naives) CHC; J. Viral Hepatitis 2006
⁶Manns et al, PegIntron (Riba) vs IFN (Riba) in (CHC); Lancet 2001
⁷Poordad et al, RVR in the management of CHC: Clin Inf Dis 2008
⁸Hoofnagel et al, PegInteferon & Riba case study; NEJM 2008
⁹Schering Plough Treatment Outcomes Study

Of the endpoints related to SOC therapy (interferon-α and ribavirin) in the table above, EVR represents the most important negative predictor of outcome. Patients failing to achieve an EVR (>2 log 10 reduction in viral load) by week 12 on interferon therapy have <3% chance of ultimately achieving an SVR. These patients are routinely taken off therapy to spare them from the significant side effects associated with SOC, since it is believed that the native immune response in these patients is incapable of clearing virally infected cells in the context of 48 weeks of viral suppression. RVR and cEVR are positive predictive endpoints, with approximately 90% of patients ultimately achieving SVR after 48 weeks of pegylated-interferon-based therapy.

Patients are categorized by their response at these virologic endpoints. “Null Responders” are patients that cannot achieve at least a 1 log₁₀ reduction in viral load by week 12 on SOC; it is believed that these patients may have an impaired immune system. “Non-Responders” are patients who receive a 12-week course of therapy and fail to achieve EVR. “Partial Responders” are defined as patients who have >2 log₁₀ viral load reduction by 12 weeks, but never achieve viral negativity. These patients have a 20-30% chance of responding to a more aggressive regimen. “Relapsers” are patients who achieve viral eradication (negativity) at end of treatment, but whose viral load returns to detectable levels during the 24 week follow up.

The average patient response to 48 weeks of standard of care in genotype 1 patients has been well characterized. For example, of patients with chronic hepatitis C infection (genotype 1) receiving the SOC therapy of pegylated interferon-α2 (PEGASYS® (Peginterferon alfa-2a; Roche Pharmaceuticals)) plus ribavirin, the following table shows the typical expected response for these patients.

| | Interferon/Ribavirin Treatment Phenotype of Patient | | |
|---|---|---|---|
| Response Endpoint | Naïve | Relapser | Non-Responder |
| RVR | 10-15%¹ | | |
| EVR | ~80%¹ | 57%² | 33%² |
| cEVR | ~43%³ | | |
| ETR | 68-69%^{4,5,6} | | |
| SVR24 | 46-52%^{4,6,7,8} | | 10-15%⁹ |

¹Schiffman M L (2007) “New Management Strategies for HCV Nonrespenders and Relapsers”
²Sporea et al, Randomized Study of Pegasys (Riba) vs PegIntron (Riba); J Gastro Liver Disease, June 2006
³PROVE 2 study; taken from DM Stakeholder Opinions (Datamonitor Stakeholder Opinions: Hepatitis C “Small molecule antivirals pave the way for triple therapy” December 2007) - 12 wks of triple therapy
⁴Schiffman et al, Pegasys (Riba) v PegIntron (Riba) v Pegasys in CHC; NEJM 2002
⁵Poordad et al, RVR in the management of CHC: Clin Inf Dis 2008
⁶Jensen et al, Early ID of HCV G1 patients responding to 24 wks of treatment; Hepatology 2006
⁷Pegasys prescribing information 2008; Roche
⁸Brandao et al, 24 vs 48 weeks of Pegasys (Riba) in (Geno 1, naives) CHC; J. Viral Hepatitis 2006.
⁹Nevens et al. J Hepatol 2005: 42: A588

Numerous reports suggest that viral replication, the level of viremia, and progression to the chronic state in hepatitis C-infected individuals are influenced directly and indirectly by HCV-specific cellular immunity mediated by CD4+ helper (Th) and CD8+ cytotoxic T lymphocytes (CTLs) (Cooper et al., Immunity 1999; 10:439-449; Gerlac et al., Gastroenterology 1999; 117:933-941; Lechner et al., J Exp Med 2000; 191:1499-1512; Thimme et al., J Exp Med 2001; 194:1395-1406; Shoukry et al., Annual Rev Microbiol 2004; 58:391-424). Studies of humans and chimpanzees have revealed that HCV can replicate for weeks before the onset of CD4+ and CD8+ T cell responses can be detected in the liver and in the blood. Moreover, there may be a delay in the acquisition of function by CD8+ (and perhaps CD4+) T cells even after their expansion in blood (Shoukry, ibid.). The appearance of functional CD8+ T cells is kinetically associated with control of viremia and, at least in some cases, with an elevation in serum transaminases, suggesting that liver damage during acute hepatitis C is immunopathological. At highest risk of persistent HCV infection are those individuals who fail to generate a detectable virus-specific T lymphocyte response in the blood, liver, or both. Perhaps most importantly, generation of a cellular immune response does not necessarily ensure that the infection will be permanently controlled. CD4+ and CD8+ T cell responses must be sustained for weeks or months beyond the point of apparent control of virus replication to prevent relapse and establishment of a persistent infection.

### Immunotherapeutic Compositions

The present invention includes the use of at least one immunotherapeutic composition. In one aspect, the immunotherapeutic composition elicits a CD8+ T cell response. In one aspect, the immunotherapeutic composition elicits a CD4+ T cell response. In one aspect, the immunotherapeutic composition elicits a CD4+ T cell response and a CD8+ T cell response. In one aspect, the immunotherapeutic composition has one or more of the following characteristics: (a) stimulates one or more pattern recognition receptors effective to activate an antigen presenting cell; (b) upregulates adhesion molecules, co-stimulatory molecules, and MHC class I and/or class II molecules on antigen presenting cells; (c) induces production of proinflammatory cytokines by antigen presenting cells; (d) induces production of Th1-type cytokines by T cells; (e) induces production of Th17-type cytokines by T cells; (f) inhibits or downregulates Treg; and/or (g) elicits MHC Class I and/or MHC Class II, antigen-specific immune responses. Suitable immunotherapeutic compositions can include yeast-based immunotherapy compositions, viral-based immunotherapy compositions, antibody-based immunotherapy compositions, DNA immunotherapy compositions, subunit vaccines, and any components or adjuvants useful for stimulating or modulating an immune response, such as TLR agonists, cytokines, immune potentiators, and other agents, many of which are described in more detail below.

### Yeast-Based Immunotherapy Compositions

In one aspect of any embodiment of the invention, the invention includes the use of at least one “yeast-based immunotherapeutic composition” (which phrase may be used interchangeably with “yeast-based immunotherapy product”, “yeast-based composition”, “yeast-based immunotherapeutic” or “yeast-based vaccine”). As used herein, the phrase “yeast-based immunotherapy” or “yeast-based immunotherapy composition” refers to a composition (or use of such composition) that includes a yeast vehicle component and that elicits an immune response sufficient to achieve at least one therapeutic benefit in a subject. More particularly, a yeast-based immunotherapeutic composition is a composition that includes a yeast vehicle component and can elicit or induce an immune response, such as a cellular immune response, including without limitation a T cell-mediated cellular immune response. In one aspect, a yeast-based immunotherapy composition useful in the invention is capable of inducing a CD8+ and/or a CD4+ T cell-mediated immune response and in one aspect, a CD8+ and a CD4+ T cell-mediated immune response. Optionally, a yeast-based immunotherapy composition is capable of eliciting a humoral immune response. A yeast-based immunotherapy composition useful in the present invention can, for example, elicit an immune response in an individual such that the individual is treated for the disease or condition, or from symptoms resulting from the disease or condition.

Yeast-based immunotherapy compositions of the invention may be either “prophylactic” or “therapeutic”. When provided prophylactically, the immunotherapy compositions of the present invention are provided in advance of any symptom of a disease or condition. The prophylactic administration of the immunotherapy compositions serves to prevent or ameliorate or delay time to onset of any subsequent disease. When provided therapeutically, the immunotherapy compositions are provided at or after the onset of a symptom of disease.

Typically, a yeast-based immunotherapy composition includes a yeast vehicle and at least one antigen or immunogenic domain thereof expressed by, attached to, or mixed with the yeast vehicle. In some embodiments, the antigen or immunogenic domain thereof is provided as a fusion protein. In one aspect of the invention, fusion protein can include two or more antigens. In one aspect, the fusion protein can include two or more immunogenic domains of one or more antigens, or two or more epitopes of one or more antigens.

In any of the yeast-based immunotherapy compositions used in the present invention, the following aspects related to the yeast vehicle are included in the invention. According to the present invention, a yeast vehicle is any yeast cell (e.g., a whole or intact cell) or a derivative thereof (see below) that can be used in conjunction with one or more antigens, immunogenic domains thereof or epitopes thereof in a therapeutic composition of the invention, or in one aspect, the yeast vehicle can be used alone or as an adjuvant. The yeast vehicle can therefore include, but is not limited to, a live intact yeast microorganism (i.e., a yeast cell having all its components including a cell wall), a killed (dead) or inactivated intact yeast microorganism, or derivatives thereof including: a yeast spheroplast (i.e., a yeast cell lacking a cell wall), a yeast cytoplast (i.e., a yeast cell lacking a cell wall and nucleus), a yeast ghost (i.e., a yeast cell lacking a cell wall, nucleus and cytoplasm), a subcellular yeast membrane extract or fraction thereof (also referred to as a yeast membrane particle and previously as a subcellular yeast particle), any other yeast particle, or a yeast cell wall preparation.

Yeast spheroplasts are typically produced by enzymatic digestion of the yeast cell wall. Such a method is described, for example, in Franzusoff et al., 1991, Meth. Enzymol. 194, 662-674., incorporated herein by reference in its entirety.

Yeast cytoplasts are typically produced by enucleation of yeast cells. Such a method is described, for example, in Coon, 1978, Natl. Cancer Inst. Monogr. 48, 45-55 incorporated herein by reference in its entirety.

Yeast ghosts are typically produced by resealing a permeabilized or lysed cell and can, but need not, contain at least some of the organelles of that cell. Such a method is described, for example, in Franzusoff et al., 1983, J. Biol. Chem. 258, 3608-3614 and Bussey et al., 1979, Biochim. Biophys. Acta 553, 185-196, each of which is incorporated herein by reference in its entirety.

A yeast membrane particle (subcellular yeast membrane extract or fraction thereof) refers to a yeast membrane that lacks a natural nucleus or cytoplasm. The particle can be of any size, including sizes ranging from the size of a natural yeast membrane to microparticles produced by sonication or other membrane disruption methods known to those skilled in the art, followed by resealing. A method for producing subcellular yeast membrane extracts is described, for example, in Franzusoff et al., 1991, Meth. Enzymol. 194, 662-674. One may also use fractions of yeast membrane particles that contain yeast membrane portions and, when the antigen or other protein was expressed recombinantly by the yeast prior to preparation of the yeast membrane particles, the antigen or other protein of interest. Antigens or other proteins of interest can be carried inside the membrane, on either surface of the membrane, or combinations thereof (i.e., the protein can be both inside and outside the membrane and/or spanning the membrane of the yeast membrane particle). In one embodiment, a yeast membrane particle is a recombinant yeast membrane particle that can be an intact, disrupted, or disrupted and resealed yeast membrane that includes at least one desired antigen or other protein of interest on the surface of the membrane or at least partially embedded within the membrane.

An example of a yeast cell wall preparation is isolated yeast cell walls carrying an antigen on its surface or at least partially embedded within the cell wall such that the yeast cell wall preparation, when administered to an animal, stimulates a desired immune response against a disease target.

Any yeast strain can be used to produce a yeast vehicle of the present invention. Yeast are unicellular microorganisms that belong to one of three classes: Ascomycetes, Basidiomycetes and Fungi Imperfecti. One consideration for the selection of a type of yeast for use as an immune modulator is the pathogenicity of the yeast. In one embodiment, the yeast is a non-pathogenic strain such as Saccharomyces cerevisiae. The selection of a non-pathogenic yeast strain minimizes any adverse effects to the individual to whom the yeast vehicle is administered. However, pathogenic yeast may be used if the pathogenicity of the yeast can be negated by any means known to one of skill in the art (e.g., mutant strains). In accordance with one aspect of the present invention, nonpathogenic yeast strains are used.

Genera of yeast strains that may be used in the invention include but are not limited to Saccharomyces, Candida (which can be pathogenic), Cryptococcus, Hansenula, Kluyveromyces, Pichia, Rhodotorula, Schizosaccharomyces and Yarrowia. In one aspect, yeast genera are selected from Saccharomyces, Candida, Hansenula, Pichia or Schizosaccharomyces, and in one aspect, Saccharomyces is used. Species of yeast strains that may be used in the invention include but are not limited to Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia lipolytica. It is to be appreciated that a number of these species include a variety of subspecies, types, subtypes, etc. that are intended to be included within the aforementioned species. In one aspect, yeast species used in the invention include S. cerevisiae, C. albicans, H. polymorpha, P. pastoris and S. pombe. S. cerevisiae is useful due to it being relatively easy to manipulate and being “Generally Recognized As Safe” or “GRAS” for use as food additives (GRAS, FDA proposed Rule 62FR18938, Apr. 17, 1997). One embodiment of the present invention is a yeast strain that is capable of replicating plasmids to a particularly high copy number, such as a S. cerevisiae cir° strain. The S. cerevisiae strain is one such strain that is capable of supporting expression vectors that allow one or more target antigen(s) and/or antigen fusion protein(s) and/or other proteins to be expressed at high levels. In addition, any mutant yeast strains can be used in the present invention, including those that exhibit reduced post-translational modifications of expressed target antigens or other proteins, such as mutations in the enzymes that extend N-linked glycosylation.

In one embodiment, a yeast vehicle of the present invention is capable of fusing with the cell type to which the yeast vehicle and antigen/agent is being delivered, such as a dendritic cell or macrophage, thereby effecting particularly efficient delivery of the yeast vehicle, and in many embodiments, the antigen(s) or other agent, to the cell type. As used herein, fusion of a yeast vehicle with a targeted cell type refers to the ability of the yeast cell membrane, or particle thereof, to fuse with the membrane of the targeted cell type (e.g., dendritic cell or macrophage), leading to syncytia formation. As used herein, a syncytium is a multinucleate mass of protoplasm produced by the merging of cells. A number of viral surface proteins (including those of immunodeficiency viruses such as HIV, influenza virus, poliovirus and adenovirus) and other fusogens (such as those involved in fusions between eggs and sperm) have been shown to be able to effect fusion between two membranes (i.e., between viral and mammalian cell membranes or between mammalian cell membranes). For example, a yeast vehicle that produces an HIV gp120/gp41 heterologous antigen on its surface is capable of fusing with a CD4+ T-lymphocyte. It is noted, however, that incorporation of a targeting moiety into the yeast vehicle, while it may be desirable under some circumstances, is not necessary. In the case of yeast vehicles that express antigens extracellularly, this can be a further advantage of the yeast vehicles of the present invention. In general, yeast vehicles useful in the present invention are readily taken up by dendritic cells (as well as other cells, such as macrophages).

In most embodiments of the invention, the yeast-based immunotherapy composition includes at least one antigen, immunogenic domain thereof, or epitope thereof. The antigens contemplated for use in this invention include any antigen against which it is desired to elicit an immune response.

The antigens contemplated for use in this invention include any antigens associated with a pathogen or a disease or condition caused by or associated with a pathogen. Such antigens include, but are not limited to, any antigens associated with a pathogen, including viral antigens, fungal antigens, bacterial antigens, helminth antigens, parasitic antigens, ectoparasite antigens, protozoan antigens, or antigens from any other infectious agent. These antigens can be native antigens (with respect to the organism from which they are derived) or genetically engineered antigens which have been modified in some manner (e.g., sequence change or generation of a fusion protein). It will be appreciated that in some embodiments (i.e., when the antigen is expressed by the yeast vehicle from a recombinant nucleic acid molecule), the antigen can be a protein or any epitope or immunogenic domain thereof, a fusion protein, or a chimeric protein, rather than an entire cell or microorganism.

In one aspect, the antigen is from virus, including, but not limited to, adenoviruses, arena viruses, bunyaviruses, coronaviruses, coxsackie viruses, cytomegaloviruses, Epstein-Barr viruses, flaviviruses, hepadnaviruses, hepatitis viruses (including HCV and HBV), herpes viruses, influenza viruses, lentiviruses, measles viruses, mumps viruses, myxoviruses, orthomyxoviruses, papilloma viruses, papovaviruses, parainfluenza viruses, paramyxoviruses, parvoviruses, picornaviruses, pox viruses, rabies viruses, respiratory syncytial viruses, reoviruses, rhabdoviruses, rubella viruses, togaviruses, and varicella viruses. Other viruses include T-lymphotrophic viruses, such as human T-cell lymphotrophic viruses (HTLVs, such as HTLV-I and HTLV-II), bovine leukemia viruses (BLVS) and feline leukemia viruses (FLVs). The lentiviruses include, but are not limited to, human (HIV, including HIV-1 or HIV-2), simian (SIV), feline (FIV) and canine (CIV) immunodeficiency viruses. In one embodiment, viral antigens include those from non-oncogenic viruses.

In one embodiment of the invention, the compositions of the invention include at least one HCV antigen and/or at least one immunogenic domain of at least one HCV antigen for immunizing a subject. The composition can include, one, two, a few, several or a plurality of HCV antigens, including one or more immunogenic domains of one or more HCV antigens, as desired. For example, any protein, including any fusion protein, described herein can include at least one or more portions of any one or more HCV proteins selected from: HCV E1 envelope glycoprotein, HCV E2 envelope glycoprotein, HCV P7 ion channel, HCV NS2 metalloprotease, HCV NS3 protease/helicase, HCV NS4a NS3 protease cofactor, HCV NS4b, HCV NS5a, HCV NS5b RNA-dependent RNA polymerase, and HCV Core sequence. In one aspect, the fusion protein comprises at least one or more immunogenic domains of one or more HCV antigens.

In one preferred aspect of the invention, the HCV antigen is an HCV protein consisting of HCV NS3 protease and Core sequence. In another aspect, the HCV antigen consists of an HCV NS3 protein lacking the catalytic domain of the natural NS3 protein which is linked to HCV Core sequence. In another aspect, the HCV antigen consists of the 262 amino acids of HCV NS3 following the initial N-terminal 88 amino acids of the natural NS3 protein (i.e., positions 89-350 of HCV NS3; SEQ ID NO:20) linked to HCV Core sequence. In one aspect, the HCV Core sequence lacks the hydrophobic C-terminal sequence. In another aspect, the HCV Core sequence lacks the C-terminal two amino acids, glutamate and aspartate. In a preferred aspect, the HCV Core sequence consists of amino acid positions 2 through 140 of the natural HCV Core sequence.

For example, a yeast (e.g., Saccharomyces cerevisiae) was engineered to express a HCV NS3-Core fusion protein under the control of the copper-inducible promoter, CUP1. The fusion protein is a single polypeptide with the following sequence elements fused in frame from N- to C-terminus (HCV polyprotein (SEQ ID NO:20) numbering in parentheses, with the amino acid sequence of the fusion protein being represented herein by SEQ ID NO:2): 1) the sequence MADEAP (SEQ ID NO:9) to impart resistance to proteasomal degradation (positions 1 to 6 of SEQ ID NO:2); 2) amino acids 89 to 350 of (1115 to 1376 of SEQ ID NO:20) of the HCV NS3 protease protein (positions 6 to 268 of SEQ ID NO:2); 3) a single threonine amino acid residue introduced in cloning (position 269 of SEQ ID NO:2); 4) amino acids 2 to 140 (2 to 140 of SEQ ID NO:20) of the HCV Core protein (positions 270 to 408 of SEQ ID NO:2); and 5) the sequence E-D to increase the hydrophilicity of the Core variant (positions 409 to 410 of SEQ ID NO:2). A nucleic acid sequence encoding the fusion protein of SEQ ID NO:2 is represented herein by SEQ ID NO:1. SEQ ID NO:2 is the fusion protein expressed by the yeast-based immunotherapy product referred to herein as GI-5005.

In another preferred aspect of the invention, the HCV antigen is an inactivated full-length HCV NS3 that is part of a fusion protein according to the invention. In this embodiment, a yeast (e.g., Saccharomyces cerevisiae) was engineered to express an inactivated full-length HCV NS3 fusion protein under the control of the copper-inducible promoter, CUP1. The fusion protein comprising the full-length HCV NS3 is a single polypeptide with the following sequence elements fused in frame from N- to C-terminus (HCV polyprotein numbering in parentheses, with the amino acid sequence of the fusion protein being represented herein by SEQ ID NO:4): 1) the sequence MADEAP (SEQ ID NO:9) to impart resistance to proteasomal degradation (positions 1 to 6 of SEQ ID NO:4); and 2) amino acids 1 to 631 (1027 to 1657 of SEQ ID NO:20) of the HCV NS3 protease protein (positions 7 to 637 of SEQ ID NO:4) (note that the amino acid at HCV polypeptide residue 1165 has been changed from a serine to an alanine in order to inactivate the proteolytic activity). A nucleic acid sequence encoding the fusion protein of SEQ ID NO:4 is represented herein by SEQ ID NO:3.

In another preferred aspect of the invention, the yeast composition comprises a truncated HCV E1-E2 fusion protein. In this embodiment, a yeast (e.g., Saccharomyces cerevisiae) is engineered to express an E1-E2 fusion protein as a single polypeptide having the following sequence elements fused in frame from N- to C-terminus (HCV polyprotein numbering in parentheses, where the amino acid sequence of the fusion protein is represented herein by SEQ ID NO:6): 1) The sequence MADEAP (SEQ ID NO:9) to impart resistance to proteasomal degradation (positions 1 to 6 of SEQ ID NO:6); 2) amino acids 1 to 156 (192 to 347 of SEQ ID NO:20) of HCV protein E1 (positions 7 to 162 of SEQ ID NO:6); and 3) amino acids 1 to 334 (384 to 717 of SEQ ID NO:20) of HCV protein E2 (positions 163 to 446 of SEQ ID NO:6). It is noted that in this particular fusion protein, 36 C-terminal hydrophobic amino acids of E1 and 29 C-terminal hydrophobic amino acids of E2 were omitted from the fusion protein to promote cytoplasmic accumulation in yeast. A nucleic acid sequence encoding the fusion protein of SEQ ID NO:6 is represented herein by SEQ ID NO:5.

In yet another preferred aspect of the invention, the yeast composition comprises a transmembrane (TM) domain-deleted HCV NS4b fusion protein. The fusion protein is a single polypeptide with the following sequence elements arranged in tandem, in frame, from N- to C-terminus (polyprotein numbering in parentheses, with the amino acid sequence of the fusion protein being represented herein by SEQ ID NO:8): 1) The sequence MADEAP (SEQ ID NO:9) to impart resistance to proteosomal degradation (positions 1 to 6 of SEQ ID NO:8); 2) amino acids 1 to 69 (1712 to 1780 of SEQ ID NO:20) of HCV protein NS4b (positions 7 to 75 of SEQ ID NO:8); and 3) amino acids 177 to 261 (1888 to 1972 of SEQ ID NO:20) of HCV protein NS4b (positions 76 to 160 of SEQ ID NO:8). A 107 amino acid region corresponding to NS4b amino acids 70 to 176 (1781 to 1887 of SEQ ID NO:20) that contains multiple membrane spanning domains was omitted to promote cytoplasmic accumulation in yeast. A nucleic acid sequence encoding the fusion protein of SEQ ID NO:8 is represented herein by SEQ ID NO:7.

In yet another preferred aspect of the invention, the yeast composition comprises a Core-E1-E2 fusion protein. The fusion protein is a single polypeptide with the following sequence elements arranged in tandem, in frame, from N- to C-terminus (polyprotein numbering in parentheses, with the amino acid sequence of the fusion protein being represented herein by SEQ ID NO:12): 1) The sequence MADEAP (SEQ ID NO:9) to impart resistance to proteosomal degradation (positions 1-6 of SEQ ID NO:12); and 2) amino acids 1 to 746 (2 to 746 of SEQ ID NO:20) of unmodified HCV polyprotein encoding full-length Core, E1, and E2 proteins (positions 7 to 751 of SEQ ID NO:12: Core spanning from position 7 to 196; E1 spanning from positions 197 to 387; and E2 spanning from positions 388 to 751). A nucleic acid sequence encoding the fusion protein of SEQ ID NO:12 is represented herein by SEQ ID NO:11.

In another preferred aspect of the invention, the yeast composition comprises a Core-E1-E2 fusion protein with transmembrane domains deleted. The fusion protein is a single polypeptide with the following sequence elements fused in frame from N- to C-terminus (polyprotein numbering in parentheses, with the amino acid sequence of the fusion protein being represented herein by SEQ ID NO:14): 1) The sequence MADEAP (SEQ ID NO:9) to impart resistance to proteasomal degradation, 2) amino acids 2 to 140 (2 to 140 of SEQ ID NO:20) of HCV Core protein (positions 7 to 145 of SEQ ID NO:14), 3) amino acids 1 to 156 (192 to 347 of SEQ ID NO:20) of HCV protein E1 (positions 146 to 301 of SEQ ID NO:14), and 4) amino acids 1 to 334 (384 to 717 of SEQ ID NO:20) of HCV protein E2 (positions 302 to 635 of SEQ ID NO:14). The 51 C-terminal hydrophobic amino acids of Core protein, the 36 C-terminal hydrophobic amino acids of E1 and the 29 C-terminal hydrophobic amino acids of E2 were omitted from the fusion protein to promote cytoplasmic accumulation in yeast. A nucleic acid sequence encoding the fusion protein of SEQ ID NO:14 is represented herein by SEQ ID NO:13.

In yet another preferred aspect of the invention, the yeast composition comprises an N53-NS4a-NS4b fusion protein wherein the NS3 protease is inactivated and the NS4b lacks a transmembrane domain. The NS3-NS4a-NS4b fusion protein is a single polypeptide with the following sequence elements fused in frame from N- to C-terminus (polyprotein numbering in parentheses, with the amino acid sequence of the fusion protein being represented herein by SEQ ID NO:16): 1) The sequence MADEAP (SEQ ID NO:9) to impart resistance to proteasomal degradation (positions 1 to 6 of SEQ ID NO:16); 2) amino acids 1 to 631 (1027 to 1657 of SEQ ID NO:20) corresponding to full-length HCV NS3 protein (note: Serine 139 (position 1165, with respect to SEQ ID NO:20) is changed to alanine to inactivate the proteolytic potential of NS3) (positions 7 to 634 of SEQ ID NO:16); 3) amino acids 1 to 54 (1658 to 1711 of SEQ ID NO:20) of NS4a protein (positions 635 to 691 of SEQ ID NO:16); 4) amino acids 1 to 69 (1712 to 1780 of SEQ ID NO:20) of HCV protein NS4b (positions 692 to 776 of SEQ ID NO:16); and 5) amino acids 177 to 261 (1888 to 1972 of SEQ ID NO:20) of HCV protein NS4b (positions 777 to 845 of SEQ ID NO:16). A 107 amino acid region corresponding to NS4b amino acids 70 to 176 (1781 to 1887 of SEQ ID NO:20) that contains multiple membrane spanning domains was omitted to promote cytoplasmic accumulation in yeast. A nucleic acid sequence encoding the fusion protein of SEQ ID NO:16 is represented herein by SEQ ID NO:15.

In another preferred aspect of the invention, the yeast composition comprises a NS5a-NS5b fusion protein with an inactivating deletion of NS5b C-terminus. This NS5a-NS5b fusion protein is a single polypeptide with the following sequence elements fused in frame from N- to C-terminus (polyprotein numbering in parentheses, with the amino acid sequence of the fusion protein being represented herein by SEQ ID NO:18): 1) The sequence MADEAP (SEQ ID NO:9) to impart resistance to proteasomal degradation (positions 1 to 6 of SEQ ID NO:18); 2) the entirety of NS5a protein corresponding to amino acids 1 to 448 (1973 to 2420 of SEQ ID NO:20) (positions 7 to 454 of SEQ ID NO:18); and 3) amino acids 1 to 539 (2421 to 2959 of SEQ ID NO:20) of NS5b (positions 455 to 993 of SEQ ID NO:18). The 52 C-terminal residues that are required for the activity of NS5b in HCV replication were deleted to inactivate the protein. A nucleic acid sequence encoding the fusion protein of SEQ ID NO:18 is represented herein by SEQ ID NO:17.

In a particular aspect of the invention, the above-described fusion proteins contain one or more heterologous linker sequences between two HCV proteins (e.g., the HCV NS3 sequence and the HCV Core sequence). In a preferred embodiment, the heterologous linker sequence consists of a single heterologous amino acid residue. In a more preferred embodiment, the heterologous linker sequence consists of a single threonine residue.

In another aspect of the invention, the compositions of the invention include at least one HBV antigen and/or at least one immunogenic domain of at least one HBV antigen for immunizing a subject. The composition can include, one, two, a few, several or a plurality of HBV antigens, including one or more immunogenic domains of one or more HBV antigens, as desired. For example, any protein, including any fusion protein, described herein can include at least one or more portions of any one or more HBV proteins selected from: HbsAg, Pol, Core and X proteins. In one aspect, the fusion protein comprises at least one or more immunogenic domains of one or more HBV antigens. An HBV protein or fusion protein encompassed by the invention can include at least a portion or the full-length of any one or more HBV proteins selected from: HBV surface protein (also called surface antigen or envelope protein or HBsAg), including the large (L), middle (M) and/or small (S) forms of surface protein; HBV precore protein; HBV core protein (also called core antigen or HBcAg); HBV e-antigen (also called HBeAg); HBV polymerase (including one or both domains of the polymerase, called the RT domain and the TP domain); HBV X antigen (also called X or HBx); and/or any one or more immunogenic domains of any one or more of these HBV proteins.

Combinations of HBV antigens useful in the present invention include, but are not limited to (in any order within the fusion):

(1) surface protein (L, M and/or S and/or any one or combination of functional and/or immunological domains thereof) in combination with any one or more of: (a) precore/core/e (precore, core, e-antigen, and/or any one or combination of functional and/or immunological domains thereof); (b) polymerase (full-length, RT domain, TP domain and/or any one or combination of functional and/or immunological domains thereof); and/or (c) X antigen (or any one or combination of functional and/or immunological domains thereof);

(2) precore/core/e (precore, core, e-antigen, and/or any one or combination of functional and/or immunological domains thereof) in combination with any one or more of: (a) surface protein (L, M and/or S and/or any one or combination of functional and/or immunological domains thereof); (b) polymerase (full-length, RT domain, TP domain and/or any one or combination of functional and/or immunological domains thereof); and/or (c) X antigen (or any one or combination of functional and/or immunological domains thereof);

(3) polymerase (full-length, RT domain, TP domain and/or any one or combination of functional and/or immunological domains thereof) in combination with any one or more of: (a) surface protein (L, M and/or S and/or any one or combination of functional and/or immunological domains thereof); (b) precore/core/e (precore, core, e-antigen, and/or any one or combination of functional and/or immunological domains thereof); and/or (c) X antigen (or any one or combination of functional and/or immunological domains thereof); and/or

(4) X antigen (or any one or combination of functional and/or immunological domains thereof) in combination with any one or more of: (a) surface protein (L, M and/or S and/or any one or combination of functional and/or immunological domains thereof); (b) polymerase (full-length, RT domain, TP domain and/or any one or combination of functional and/or immunological domains thereof); and/or (c) precore/core/e (precore, core, e-antigen, and/or any one or combination of functional and/or immunological domains thereof).

The nucleic acid and amino acid sequence for HBV genes and the proteins encoded thereby are known in the art for each of the known genotypes. The table below provides reference to sequence identifiers for exemplary (representative) amino acid sequences of all of the HBV structural and non-structural proteins in each of the eight known genotypes of HBV, and further indicates certain structural domains. It is noted that small variations may occur in the amino acid sequence between different viral isolates of the same protein from the same HBV genotype. However, as discussed above, strains and serotypes of HBV and genotypes of HBV display high amino acid identity even between serotypes and genotypes. Therefore, using the guidance provided herein and the reference to the exemplary HBV sequences, one of skill in the art will readily be able to produce a variety of HBV-based proteins and/or homologues thereof, including fusion proteins, from any HBV strain, serotype, or genotype, for use in the compositions and methods of the present invention.

| Organism, | | Sequence Identifier |
|---|---|---|
| Genotype, Gene | Protein | (Database Accession No.) |
| HBV, Genotype | Precore | SEQ ID NO: 24 |
| A, C | | (Accession No. AAX83988.1) |
| | Core (HBcAg) | *Positions 30/31-212 of |
| | | SEQ ID NO: 24 |
| | e-antigen | *Positions 20-178 of |
| | (HBeAg) | SEQ ID NO: 24 |
| HBV, Genotype | Polymerase | SEQ ID NO: 25 |
| A, P | | (Accession No. BAI81985) |
| | reverse | *Positions 383-602 of |
| | transcriptase | SEQ ID NO: 25 |
| HBV, Genotype | Surface HBsAg | SEQ ID NO: 26 |
| A, S | (L) | (Accession No. BAD91280.1) |
| | Surface HBsAg | *Positions 120-400 of |
| | (M) | SEQ ID NO: 26 |
| | Surface HBsAg | *Positions 175-400 of |
| | (S) | SEQ ID NO: 26 |
| HBV, Genotype | X (HBx) | SEQ ID NO: 27 |
| A, X | | (Accession No. AAK97189.1) |
| HBV, Genotype | Precore | SEQ ID NO: 28 |
| B, C | | (Accession No. BAD90067) |
| | Core (HBcAg) | *Positions 30/31-212 of |
| | | SEQ ID NO: 28 |
| | e-antigen | *Positions 20-178 of |
| | (HBeAg) | SEQ ID NO: 28 |
| HBV, Genotype | Polymerase | SEQ ID NO: 29 |
| B, P | | (Accession No. BAD90068.1) |
| | reverse | *Positions 381-600 of |
| | transcriptase | SEQ ID NO: 29 |
| HBV, Genotype | Surface HBsAg | SEQ ID NO: 30 |
| B, S | (L) | (Accession No. BAJ06634.1) |
| | Surface HBsAg | *Positions 120-400 of |
| | (M) | SEQ ID NO: 30 |
| | Surface HBsAg | *Positions 175-400 of |
| | (S) | SEQ ID NO: 30 |
| HBV, Genotype | X (HBx) | SEQ ID NO: 31 |
| B, X | | (Accession No. BAD90066.1) |
| HBV, Genotype | Precore | SEQ ID NO: 32 |
| C, C | | (Accession No. YP_355335) |
| | Core (HBcAg) | *Positions 30/31-212 of |
| | | SEQ ID NO: 32 |
| | e-antigen | *Positions 20-178 of |
| | (HBeAg) | SEQ ID NO: 32 |
| HBV, Genotype | Polymerase | SEQ ID NO: 33 |
| C, P | | (Accession No. ACH57822) |
| | reverse | *Positions 381-600 of |
| | transcriptase | SEQ ID NO: 33 |
| HBV, Genotype | Surface HBsAg | SEQ ID NO: 34 |
| C, S | (L) | (Accession No. BAJ06646.1) |
| | Surface HBsAg | *Positions 120-400 of |
| | (M) | SEQ ID NO: 34 |
| | Surface HBsAg | *Positions 175-400 of |
| | (S) | SEQ ID NO: 34 |
| HBV, Genotype | X (HBx) | SEQ ID NO: 35 |
| C, X | | (Accession No. BAJ06639.1) |
| HBV, Genotype | Precore | SEQ ID NO: 36 |
| D, C | | (Accession No. ADF29260.1) |
| | Core (HBcAg) | *Positions 30/31-212 of |
| | | SEQ ID NO: 36 |
| | e-antigen | *Positions 20-178 of |
| | (HBeAg) | SEQ ID NO: 36 |
| HBV, Genotype | Polymerase | SEQ ID NO: 37 |
| D, P | | (Accession No. ADD12642.1) |
| | reverse | *Positions 370-589 of |
| | transcriptase | SEQ ID NO: 37 |
| HBV, Genotype | Surface HBsAg | SEQ ID NO: 38 |
| D, S | (L) | (Accession No. ACP20363.1) |
| | Surface HBsAg | *Positions 109-389 of |
| | (M) | SEQ ID NO: 38 |
| | Surface HBsAg | *Positions 164-389 of |
| | (S) | SEQ ID NO: 38 |
| HBV, Genotype | X (HBx) | SEQ ID NO: 39 |
| D, X | | (Accession No. BAF47226.1) |
| HBV, Genotype | Precore | SEQ ID NO: 40 |
| E, C | | (Accession No. ACU25047.1) |
| | Core (HBcAg) | *Positions 30/31-212 of |
| | | SEQ ID NO: 40 |
| | e-antigen | *Positions 20-178 of |
| | (HBeAg) | SEQ ID NO: 40 |
| HBV, Genotype | Polymerase | SEQ ID NO: 41 |
| E, P | | (Accession No. ACO89764.1) |
| | reverse | *Positions 380-599 of |
| | transcriptase | SEQ ID NO: 41 |
| HBV, Genotype | Surface HBsAg | SEQ ID NO: 42 |
| E, S | (L) | (Accession No. BAD91274.1) |
| | Surface HBsAg | *Positions 119-399 of |
| | (M) | SEQ ID NO: 42 |
| | Surface HBsAg | *Positions 174-399 of |
| | (S) | SEQ ID NO: 42 |
| HBV, Genotype | X (HBx) | SEQ ID NO: 43 |
| E, X | | (Accession No. ACU24870.1) |
| HBV, Genotype | Precore | SEQ ID NO: 44 |
| F, C | | (Accession No. BAB17946.1) |
| | Core (HBcAg) | *Positions 30/31-212 of |
| | | SEQ ID NO: 44 |
| | e-antigen | *Positions 20-178 of |
| | (HBeAg) | SEQ ID NO: 44 |
| HBV, Genotype | Polymerase | SEQ ID NO: 45 |
| F, P | | (Accession No. ACD03788.2) |
| | reverse | *Positions 381-600 of |
| | transcriptase | SEQ ID NO: 45 |
| HBV, Genotype | Surface HBsAg | SEQ ID NO: 46 |
| F, S | (L) | (Accession No. BAD98933.1) |
| | Surface HBsAg | *Positions 120-400 of |
| | (M) | SEQ ID NO: 46 |
| | Surface HBsAg | *Positions 175-400 of |
| | (S) | SEQ ID NO: 46 |
| HBV, Genotype | X (HBx) | SEQ ID NO: 47 |
| F, X | | (Accession No. AAM09054.1) |
| HBV, Genotype | Precore | SEQ ID NO: 48 |
| G, C | | (Accession No. ADD62622.1) |
| | Core (HBcAg) | *Positions 14-194 of |
| | | SEQ ID NO: 48 |
| | e-antigen | *Positions 4-161 of |
| | (HBeAg) | SEQ ID NO: 48 |
| HBV, Genotype | Polymerase | SEQ ID NO: 49 |
| G, P | | (Accession No. ADD62619.1) |
| | reverse | *Positions 380-599 of |
| | transcriptase | SEQ ID NO: 49 |
| HBV, Genotype | Surface | SEQ ID NO: 50 |
| G, S | (HBsAg) (L) | (Accession No. ADD62620.1) |
| | Surface HBsAg | *Positions 119-399 of |
| | (M) | SEQ ID NO: 50 |
| | Surface HBsAg | *Positions 174-399 of |
| | (S) | SEQ ID NO: 50 |
| HBV, Genotype | X (HBx) | SEQ ID NO: 51 |
| G, X | | (Accession No. BAB82400.1) |
| HBV, Genotype | Precore | SEQ ID NO: 52 |
| H, C | | (Accession No. BAD91265.1) |
| | Core (HBcAg) | *Positions 30/31-212 of |
| | | SEQ ID NO: 52 |
| | e-antigen | *Positions 20-178 of |
| | (HBeAg) | SEQ ID NO: 52 |
| HBV, Genotype | Polymerase | SEQ ID NO: 53 |
| H, P | | (Accession No. BAF49208.1) |
| | reverse | *Positions 381-600 of |
| | transcriptase | SEQ ID NO: 53 |
| HBV, Genotype | Surface HBsAg | SEQ ID NO: 54 |
| H, S | (L) | (Accession No. BAE20065.1) |
| | Surface HBsAg | *Positions 120-400 of |
| | (M) | SEQ ID NO: 54 |
| | Surface HBsAg | *Positions 175-400 of |
| | (S) | SEQ ID NO: 54 |
| HBV, Genotype | X (HBx) | SEQ ID NO: 55 |
| H, X | | (Accession No. BAF49206.1) |

In another aspect of the invention, the antigen is from an infectious agent from a genus selected from: Aspergillus, Bordatella, Brugia, Candida, Chlamydia, Coccidia, Cryptococcus, Dirofilaria, Escherichia, Francisella, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma, Vibriocholerae, and Yersinia. In one aspect, the infectious agent is selected from Plasmodium falciparum or Plasmodium vivax.

In one aspect, the antigen is from a bacterium from a family selected from: Enterobacteriaceae, Micrococcaceae, Vibrionaceae, Pasteurellaceae, Mycoplasmataceae, and Rickettsiaceae. In one aspect, the bacterium is of a genus selected from: Pseudomonas, Bordetella, Mycobacterium, Vibrio, Bacillus, Salmonella, Francisella, Staphylococcus, Streptococcus, Escherichia, Enterococcus, Pasteurella, and Yersinia. In one aspect, the bacterium is from a species selected from: Pseudomonas aeruginosa, Pseudomonas mallei, Pseudomonas pseudomallei, Bordetella pertussis, Mycobacterium tuberculosis, Mycobacterium leprae, Francisella tularensis, Vibrio cholerae, Bacillus anthracis, Salmonella enteric, Yersinia pestis, Escherichia coli and Bordetella bronchiseptica.

In one aspect, the antigen is from a fungus, such a fungus including, but not limited to, a fungus from Saccharomyces spp., Aspergillus spp., Cryptococcus spp., Coccidioides spp., Neurospora spp., Histoplasma spp., or Blastomyces spp. In one aspect, the fungus is from a species selected from: Aspergillus fumigatus, A. flavus, A. niger, A. terreus, A. nidulans, Coccidioides immitis, Coccidioides posadasii or Cryptococcus neoformans. The most common species of Aspergillus causing invasive disease include A. fumigatus, A. flavus, A. niger, A. terreus and A. nidulans, and may be found, for example, in patients who have immunosuppression or T-cell or phagocytic impairment. A. fumigatus has been implicated in asthma, aspergillomas and invasive aspergillosis. Coccidioidomycosis, also known as San Joaquin Valley Fever, is a fungal disease caused by Coccidioides immitis, and can lead to acute respiratory infections and chronic pulmonary conditions or dissemination to the meninges, bones, and joints. Cryptococcosis-associated conditions are also targeted by methods of the invention, for example, in a non-immunosuppressed or immunosuppressed subject, such as a subject who is infected with HIV.

In some embodiments, the antigen is a fusion protein. In one aspect of the invention, fusion protein can include two or more antigens. In one aspect, the fusion protein can include two or more immunogenic domains or two or more epitopes of one or more antigens. A yeast-based immunotherapeutic composition containing such antigens may provide antigen-specific immunization in a broad range of patients. For example, a multiple domain fusion protein useful in the present invention may have multiple domains, wherein each domain consists of a peptide from a particular protein, the peptide consisting of at least 4 amino acid residues flanking either side of and including a mutated amino acid that is found in the protein, wherein the mutation is associated with a particular disease or condition.

In one embodiment, fusion proteins that are used as a component of the yeast-based immunotherapeutic composition useful in the invention are produced using constructs that are particularly useful for the expression of heterologous antigens in yeast. Typically, the desired antigenic protein(s) or peptide(s) are fused at their amino-terminal end to: (a) a specific synthetic peptide that stabilizes the expression of the fusion protein in the yeast vehicle or prevents posttranslational modification of the expressed fusion protein (such peptides are described in detail, for example, in U.S. Patent Publication No. 2004-0156858 A1, published Aug. 12, 2004, incorporated herein by reference in its entirety); (b) at least a portion of an endogenous yeast protein, wherein either fusion partner provides significantly enhanced stability of expression of the protein in the yeast and/or a prevents post-translational modification of the proteins by the yeast cells (such proteins are also described in detail, for example, in U.S. Patent Publication No. 2004-0156858 A1, supra); and/or (c) at least a portion of a yeast protein that causes the fusion protein to be expressed on the surface of the yeast (e.g., an Aga protein, described in more detail herein). In addition, the present invention includes the use of peptides that are fused to the C-terminus of the antigen-encoding construct, particularly for use in the selection and identification of the protein. Such peptides include, but are not limited to, any synthetic or natural peptide, such as a peptide tag (e.g., 6×His) or any other short epitope tag. Peptides attached to the C-terminus of an antigen according to the invention can be used with or without the addition of the N-terminal peptides discussed above.

In one embodiment, a synthetic peptide useful in a fusion protein is linked to the N-terminus of the antigen, the peptide consisting of at least two amino acid residues that are heterologous to the antigen, wherein the peptide stabilizes the expression of the fusion protein in the yeast vehicle or prevents posttranslational modification of the expressed fusion protein. The synthetic peptide and N-terminal portion of the antigen together form a fusion protein that has the following requirements: (1) the amino acid residue at position one of the fusion protein is a methionine (i.e., the first amino acid in the synthetic peptide is a methionine); (2) the amino acid residue at position two of the fusion protein is not a glycine or a proline (i.e., the second amino acid in the synthetic peptide is not a glycine or a proline); (3) none of the amino acid residues at positions 2-6 of the fusion protein is a methionine (i.e., the amino acids at positions 2-6, whether part of the synthetic peptide or the protein, if the synthetic peptide is shorter than 6 amino acids, do not include a methionine); and (4) none of the amino acids at positions 2-6 of the fusion protein is a lysine or an arginine (i.e., the amino acids at positions 2-6, whether part of the synthetic peptide or the protein, if the synthetic peptide is shorter than 5 amino acids, do not include a lysine or an arginine). The synthetic peptide can be as short as two amino acids, but in one aspect, is at least 2-6 amino acids (including 3, 4, 5 amino acids), and can be longer than 6 amino acids, in whole integers, up to about 200 amino acids, 300 amino acids, 400 amino acids, 500 amino acids, or more.

In one embodiment, a fusion protein comprises an amino acid sequence of M-X2-X3-X4-X5-X6, wherein M is methionine; wherein X2 is any amino acid except glycine, proline, lysine or arginine; wherein X3 is any amino acid except methionine, lysine or arginine; wherein X4 is any amino acid except methionine, lysine or arginine; wherein X5 is any amino acid except methionine, lysine or arginine; and wherein X6 is any amino acid except methionine, lysine or arginine. In one embodiment, the X6 residue is a proline. An exemplary synthetic sequence that enhances the stability of expression of an antigen in a yeast cell and/or prevents post-translational modification of the protein in the yeast includes the sequence M-A-D-E-A-P (SEQ ID NO:1). In addition to the enhanced stability of the expression product, this fusion partner does not appear to negatively impact the immune response against the vaccinating antigen in the construct. In addition, the synthetic fusion peptides can be designed to provide an epitope that can be recognized by a selection agent, such as an antibody.

In one aspect of the invention, the yeast vehicle is manipulated such that the antigen is expressed or provided by delivery or translocation of an expressed protein product, partially or wholly, on the surface of the yeast vehicle (extracellular expression). One method for accomplishing this aspect of the invention is to use a spacer arm for positioning one or more protein(s) on the surface of the yeast vehicle. For example, one can use a spacer arm to create a fusion protein of the antigen(s) or other protein of interest with a protein that targets the antigen(s) or other protein of interest to the yeast cell wall. For example, one such protein that can be used to target other proteins is a yeast protein (e.g., cell wall protein 2 (cwp2), Aga2, Pir4 or Flo1 protein) that enables the antigen(s) or other protein to be targeted to the yeast cell wall such that the antigen or other protein is located on the surface of the yeast. Proteins other than yeast proteins may be used for the spacer arm; however, for any spacer arm protein, it is most desirable to have the immunogenic response be directed against the target antigen rather than the spacer arm protein. As such, if other proteins are used for the spacer arm, then the spacer arm protein that is used should not generate such a large immune response to the spacer arm protein itself such that the immune response to the target antigen(s) is overwhelmed. One of skill in the art should aim for a small immune response to the spacer arm protein relative to the immune response for the target antigen(s). Spacer arms can be constructed to have cleavage sites (e.g., protease cleavage sites) that allow the antigen to be readily removed or processed away from the yeast, if desired. Any known method of determining the magnitude of immune responses can be used (e.g., antibody production, lytic assays, etc.) and are readily known to one of skill in the art.

Another method for positioning the target antigen(s) or other proteins to be exposed on the yeast surface is to use signal sequences such as glycosylphosphatidyl inositol (GPI) to anchor the target to the yeast cell wall. Alternatively, positioning can be accomplished by appending signal sequences that target the antigen(s) or other proteins of interest into the secretory pathway via translocation into the endoplasmic reticulum (ER) such that the antigen binds to a protein which is bound to the cell wall (e.g., cwp).

In one aspect, the spacer arm protein is a yeast protein. The yeast protein can consist of between about two and about 800 amino acids of a yeast protein. In one embodiment, the yeast protein is about 10 to 700 amino acids. In another embodiment, the yeast protein is about 40 to 600 amino acids. Other embodiments of the invention include the yeast protein being at least 250 amino acids, at least 300 amino acids, at least 350 amino acids, at least 400 amino acids, at least 450 amino acids, at least 500 amino acids, at least 550 amino acids, at least 600 amino acids, or at least 650 amino acids. In one embodiment, the yeast protein is at least 450 amino acids in length.

Use of yeast proteins can stabilize the expression of fusion proteins in the yeast vehicle, prevents posttranslational modification of the expressed fusion protein, and/or targets the fusion protein to a particular compartment in the yeast (e.g., to be expressed on the yeast cell surface). For delivery into the yeast secretory pathway, exemplary yeast proteins to use include, but are not limited to: Aga (including, but not limited to, Aga1 and/or Aga2); SUC2 (yeast invertase); alpha factor signal leader sequence; CPY; Cwp2p for its localization and retention in the cell wall; BUD genes for localization at the yeast cell bud during the initial phase of daughter cell formation; Flo 1p; Pir2p; and Pir4p.

Other sequences can be used to target, retain and/or stabilize the protein to other parts of the yeast vehicle, for example, in the cytosol or the mitochondria. Examples of suitable yeast protein that can be used for any of the embodiments above include, but are not limited to, SECT; phosphoenolpyruvate carboxykinase PCK1, phosphoglycerokinase PGK and triose phosphate isomerase TPI gene products for their repressible expression in glucose and cytosolic localization; the heat shock proteins SSA1, SSA3, SSA4, SSC1, whose expression is induced and whose proteins are more thermostable upon exposure of cells to heat treatment; the mitochondrial protein CYC1 for import into mitochondria; ACT1.

Methods of producing yeast vehicles and expressing, combining and/or associating yeast vehicles with antigens and/or other proteins and/or agents of interest to produce yeast-based immunotherapy compositions are contemplated by the invention.

According to the present invention, the term “yeast vehicle-antigen complex” or “yeast-antigen complex” is used generically to describe any association of a yeast vehicle with an antigen, and can be used interchangeably with “yeast-based immunotherapy composition” when such composition is used to elicit an immune response as described above. Such association includes expression of the antigen by the yeast (a recombinant yeast), introduction of an antigen into a yeast, physical attachment of the antigen to the yeast, and mixing of the yeast and antigen together, such as in a buffer or other solution or formulation. These types of complexes are described in detail below.

In one embodiment, a yeast cell used to prepare the yeast vehicle is transfected with a heterologous nucleic acid molecule encoding a protein (e.g., the antigen or agent) such that the protein is expressed by the yeast cell. Such a yeast is also referred to herein as a recombinant yeast or a recombinant yeast vehicle. The yeast cell can then be loaded into the dendritic cell as an intact cell, or the yeast cell can be killed, or it can be derivatized such as by formation of yeast spheroplasts, cytoplasts, ghosts, or subcellular particles, any of which is followed by loading of the derivative into the dendritic cell. Yeast spheroplasts can also be directly transfected with a recombinant nucleic acid molecule (e.g., the spheroplast is produced from a whole yeast, and then transfected) in order to produce a recombinant spheroplast that expresses an antigen or other protein.

In one aspect, a yeast cell or yeast spheroplast used to prepare the yeast vehicle is transfected with a recombinant nucleic acid molecule encoding the antigen(s) or other protein such that the antigen or other protein is recombinantly expressed by the yeast cell or yeast spheroplast. In this aspect, the yeast cell or yeast spheroplast that recombinantly expresses the antigen(s) or other protein is used to produce a yeast vehicle comprising a yeast cytoplast, a yeast ghost, or a yeast membrane particle or yeast cell wall particle, or fraction thereof.

In general, the yeast vehicle and antigen(s) and/or other agents can be associated by any technique described herein. In one aspect, the yeast vehicle was loaded intracellularly with the antigen(s) and/or agent(s). In another aspect, the antigen(s) and/or agent(s) was covalently or non-covalently attached to the yeast vehicle. In yet another aspect, the yeast vehicle and the antigen(s) and/or agent(s) were associated by mixing. In another aspect, and in one embodiment, the antigen(s) and/or agent(s) is expressed recombinantly by the yeast vehicle or by the yeast cell or yeast spheroplast from which the yeast vehicle was derived.

A number of antigens and/or other proteins to be produced by a yeast vehicle of the present invention is any number of antigens and/or other proteins that can be reasonably produced by a yeast vehicle, and typically ranges from at least one to at least about 6 or more, including from about 2 to about 6 heterologous antigens and or other proteins.

Expression of an antigen or other protein in a yeast vehicle of the present invention is accomplished using techniques known to those skilled in the art. Briefly, a nucleic acid molecule encoding at least one desired antigen or other protein is inserted into an expression vector in such a manner that the nucleic acid molecule is operatively linked to a transcription control sequence in order to be capable of effecting either constitutive or regulated expression of the nucleic acid molecule when transformed into a host yeast cell. Nucleic acid molecules encoding one or more antigens and/or other proteins can be on one or more expression vectors operatively linked to one or more expression control sequences. Particularly important expression control sequences are those which control transcription initiation, such as promoter and upstream activation sequences. Any suitable yeast promoter can be used in the present invention and a variety of such promoters are known to those skilled in the art. Promoters for expression in Saccharomyces cerevisiae include, but are not limited to, promoters of genes encoding the following yeast proteins: alcohol dehydrogenase I (ADH1) or II (ADH2), CUP1, phosphoglycerate kinase (PGK), triose phosphate isomerase (TPI), translational elongation factor EF-1 alpha (TEF2), glyceraldehyde-3-phosphate dehydrogenase (GAPDH; also referred to as TDH3, for triose phosphate dehydrogenase), galactokinase (GAL1), galactose-1-phosphate uridyl-transferase (GAL7), UDP-galactose epimerase (GAL10), cytochrome c1 (CYC1), Sec7 protein (SECT) and acid phosphatase (PHO5), including hybrid promoters such as ADH2/GAPDH and CYC1/GAL10 promoters, and including the ADH2/GAPDH promoter, which is induced when glucose concentrations in the cell are low (e.g., about 0.1 to about 0.2 percent), as well as the CUP1 promoter and the TEF2 promoter. Likewise, a number of upstream activation sequences (UASs), also referred to as enhancers, are known. Upstream activation sequences for expression in Saccharomyces cerevisiae include, but are not limited to, the UASs of genes encoding the following proteins: PCK1, TPI, TDH3, CYC1, ADH1, ADH2, SUC2, GAL1, GAL7 and GAL10, as well as other UASs activated by the GAL4 gene product, with the ADH2 UAS being used in one aspect. Since the ADH2 UAS is activated by the ADR1 gene product, it may be preferable to overexpress the ADR1 gene when a heterologous gene is operatively linked to the ADH2 UAS. Transcription termination sequences for expression in Saccharomyces cerevisiae include the termination sequences of the α-factor, GAPDH, and CYC1 genes.

Transcription control sequences to express genes in methyltrophic yeast include the transcription control regions of the genes encoding alcohol oxidase and formate dehydrogenase.

Transfection of a nucleic acid molecule into a yeast cell according to the present invention can be accomplished by any method by which a nucleic acid molecule administered into the cell and includes, but is not limited to, diffusion, active transport, bath sonication, electroporation, microinjection, lipofection, adsorption, and protoplast fusion. Transfected nucleic acid molecules can be integrated into a yeast chromosome or maintained on extrachromosomal vectors using techniques known to those skilled in the art. Examples of yeast vehicles carrying such nucleic acid molecules are disclosed in detail herein. As discussed above, yeast cytoplast, yeast ghost, and yeast membrane particles or cell wall preparations can also be produced recombinantly by transfecting intact yeast microorganisms or yeast spheroplasts with desired nucleic acid molecules, producing the antigen therein, and then further manipulating the microorganisms or spheroplasts using techniques known to those skilled in the art to produce cytoplast, ghost or subcellular yeast membrane extract or fractions thereof containing desired antigens or other proteins.

Effective conditions for the production of recombinant yeast vehicles and expression of the antigen and/or other protein (e.g., an agent as described herein) by the yeast vehicle include an effective medium in which a yeast strain can be cultured. An effective medium is typically an aqueous medium comprising assimilable carbohydrate, nitrogen and phosphate sources, as well as appropriate salts, minerals, metals and other nutrients, such as vitamins and growth factors. The medium may comprise complex nutrients or may be a defined minimal medium. Yeast strains of the present invention can be cultured in a variety of containers, including, but not limited to, bioreactors, Erlenmeyer flasks, test tubes, microtiter dishes, and Petri plates. Culturing is carried out at a temperature, pH and oxygen content appropriate for the yeast strain. Such culturing conditions are well within the expertise of one of ordinary skill in the art (see, for example, Guthrie et al. (eds.), 1991, Methods in Enzymology, vol. 194, Academic Press, San Diego).

In some aspects of the invention, the yeast are grown under neutral pH conditions, and particularly, in a media maintained at a pH level of at least 5.5, namely the pH of the culture media is not allowed to drop below pH 5.5. In other aspects, the yeast is grown at a pH level maintained at about 5.5. In other aspects, the yeast is grown at a pH level maintained at about 5.6, 5.7, 5.8 or 5.9. In another aspect, the yeast is grown at a pH level maintained at about 6. In another aspect, the yeast is grown at a pH level maintained at about 6.5. In other aspects, the yeast is grown at a pH level maintained at about 6, 6.1, 6.2, 6.3, 6.4, 6.5, 6.6, 6.7, 6.8, 6.9 or 7.0. In other aspects, the yeast is grown at a pH level maintained at about 7, 7.1, 7.2, 7.3, 7.4, 7.5, 7.6, 7.7, 7.8, 7.9, or 8.0. The pH level is important in the culturing of yeast. One of skill in the art will appreciate that the culturing process includes not only the start of the yeast culture but the maintenance of the culture as well. As yeast culturing is known to turn acidic (i.e., lowering the pH) over time, care must be taken to monitor the pH level during the culturing process. Yeast cell cultures whereby the pH level of the medium drops below 6 are still contemplated within the scope of the invention provided that the pH of the media is brought up to at least 5.5 at some point during the culturing process. As such, the longer time the yeast are grown in a medium that is at least pH 5.5 or above, the better the results will be in terms of obtaining yeast with desirable characteristics.

As used herein, the general use of the term “neutral pH” refers to a pH range between about pH 5.5 and about pH 8, and in one aspect, between about pH 6 and about 8. One of skill the art will appreciate that minor fluctuations (e.g., tenths or hundredths) can occur when measuring with a pH meter. As such, the use of neutral pH to grow yeast cells means that the yeast cells are grown in neutral pH for the majority of the time that they are in culture. The use of a neutral pH in culturing yeast promotes several biological effects that are desirable characteristics for using the yeast as vehicles for immunomodulation. In one aspect, culturing the yeast in neutral pH allows for good growth of the yeast without any negative effect on the cell generation time (e.g., slowing down the doubling time). The yeast can continue to grow to high densities without losing their cell wall pliability. In another aspect, the use of a neutral pH allows for the production of yeast with pliable cell walls and/or yeast that are sensitive to cell wall digesting enzymes (e.g., glucanase) at all harvest densities. This trait is desirable because yeast with flexible cell walls can induce unusual immune responses, such as by promoting the secretion of cytokines (e.g., interferon-γ (IFN-γ)) in the cells hosting the yeast. In addition, greater accessibility to the antigens located in the cell wall is afforded by such culture methods. In another aspect, the use of neutral pH for some antigens allows for release of the di-sulfide bonded antigen by treatment with dithiothreitol (DTT) that is not possible when such an antigen-expressing yeast is cultured in media at lower pH (e.g., pH 5). Finally, in another aspect, yeast cultured using the neutral pH methodologies, elicit increased production of at least TH1-type cytokines including, but not limited to, IFN-γ, interleukin-12 (IL-12), and IL-2, and may also elicit increased production of other cytokines, such as proinflammatory cytokines (e.g., IL-6).

In one embodiment, control of the amount of yeast glycosylation is used to control the expression of antigens by the yeast, particularly on the surface. The amount of yeast glycosylation can affect the immunogenicity and antigenicity of the antigen expressed on the surface, since sugar moieties tend to be bulky. As such, the existence of sugar moieties on the surface of yeast and its impact on the three-dimensional space around the target antigen(s) should be considered in the modulation of yeast according to the invention. Any method can be used to reduce the amount of glycosylation of the yeast (or increase it, if desired). For example, one could use a yeast mutant strain that has been selected to have low glycosylation (e.g. mnn1, och1 and mnn9 mutants), or one could eliminate by mutation the glycosylation acceptor sequences on the target antigen. Alternatively, one could use a yeast with abbreviated glycosylation patterns, e.g. Pichia. One can also treat the yeast using methods that reduce or alter the glycosylation.

In one embodiment of the present invention, as an alternative to expression of an antigen or other protein recombinantly in the yeast vehicle, a yeast vehicle is loaded intracellularly with the protein or peptide, or with carbohydrates or other molecules that serve as an antigen and/or are useful as immunomodulatory agents or biological response modifiers according to the invention. Subsequently, the yeast vehicle, which now contains the antigen and/or other proteins intracellularly, can be administered to the patient or loaded into a carrier such as a dendritic cell. Peptides and proteins can be inserted directly into yeast vehicles of the present invention by techniques known to those skilled in the art, such as by diffusion, active transport, liposome fusion, electroporation, phagocytosis, freeze-thaw cycles and bath sonication. Yeast vehicles that can be directly loaded with peptides, proteins, carbohydrates, or other molecules include intact yeast, as well as spheroplasts, ghosts or cytoplasts, which can be loaded with antigens and other agents after production. Alternatively, intact yeast can be loaded with the antigen and/or agent, and then spheroplasts, ghosts, cytoplasts, or subcellular particles can be prepared therefrom. Any number of antigens and/or other agents can be loaded into a yeast vehicle in this embodiment, from at least 1, 2, 3, 4 or any whole integer up to hundreds or thousands of antigens and/or other agents, such as would be provided by the loading of a microorganism or portions thereof, for example.

In another embodiment of the present invention, an antigen and/or other agent is physically attached to the yeast vehicle. Physical attachment of the antigen and/or other agent to the yeast vehicle can be accomplished by any method suitable in the art, including covalent and non-covalent association methods which include, but are not limited to, chemically crosslinking the antigen and/or other agent to the outer surface of the yeast vehicle or biologically linking the antigen and/or other agent to the outer surface of the yeast vehicle, such as by using an antibody or other binding partner. Chemical cross-linking can be achieved, for example, by methods including glutaraldehyde linkage, photoaffinity labeling, treatment with carbodiimides, treatment with chemicals capable of linking di-sulfide bonds, and treatment with other cross-linking chemicals standard in the art. Alternatively, a chemical can be contacted with the yeast vehicle that alters the charge of the lipid bilayer of yeast membrane or the composition of the cell wall so that the outer surface of the yeast is more likely to fuse or bind to antigens and/or other agent having particular charge characteristics. Targeting agents such as antibodies, binding peptides, soluble receptors, and other ligands may also be incorporated into an antigen as a fusion protein or otherwise associated with an antigen for binding of the antigen to the yeast vehicle.

When the antigen or other protein is expressed on or physically attached to the surface of the yeast, spacer arms may, in one aspect, be carefully selected to optimize antigen or other protein expression or content on the surface. The size of the spacer arm(s) can affect how much of the antigen or other protein is exposed for binding on the surface of the yeast. Thus, depending on which antigen(s) or other protein(s) are being used, one of skill in the art will select a spacer arm that effectuates appropriate spacing for the antigen or other protein on the yeast surface. In one embodiment, the spacer arm is a yeast protein of at least 450 amino acids. Spacer arms have been discussed in detail above.

Another consideration for optimizing antigen surface expression is whether the antigen and spacer arm combination should be expressed as a monomer or as dimer or as a trimer, or even more units connected together. This use of monomers, dimers, trimers, etc. allows for appropriate spacing or folding of the antigen such that some part, if not all, of the antigen is displayed on the surface of the yeast vehicle in a manner that makes it more immunogenic.

In yet another embodiment, the yeast vehicle and the antigen or other protein are associated with each other by a more passive, non-specific or non-covalent binding mechanism, such as by gently mixing the yeast vehicle and the antigen or other protein together in a buffer or other suitable formulation (e.g., admixture).

In one embodiment of the invention, the yeast vehicle and the antigen or other protein are both loaded intracellularly into a carrier such as a dendritic cell or macrophage to form the therapeutic composition or vaccine of the present invention. Alternatively, an antigen or other protein can be loaded into a dendritic cell in the absence of the yeast vehicle.

In one embodiment, intact yeast (with or without expression of heterologous antigens or other proteins) can be ground up or processed in a manner to produce yeast cell wall preparations, yeast membrane particles or yeast fragments (i.e., not intact) and the yeast fragments can, in some embodiments, be provided with or administered with other compositions that include antigens (e.g., DNA vaccines, protein subunit vaccines, killed or inactivated pathogens) to enhance immune response. For example, enzymatic treatment, chemical treatment or physical force (e.g., mechanical shearing or sonication) can be used to break up the yeast into parts that are used as an adjuvant.

In one embodiment of the invention, yeast vehicles useful in the invention include yeast vehicles that have been killed or inactivated. Killing or inactivating of yeast can be accomplished by any of a variety of suitable methods known in the art. For example, heat inactivation of yeast is a standard way of inactivating yeast, and one of skill in the art can monitor the structural changes of the target antigen, if desired, by standard methods known in the art. Alternatively, other methods of inactivating the yeast can be used, such as chemical, electrical, radioactive or UV methods. See, for example, the methodology disclosed in standard yeast culturing textbooks such as Methods of Enzymology, Vol. 194, Cold Spring Harbor Publishing (1990). Any of the inactivation strategies used should take the secondary, tertiary or quaternary structure of the target antigen into consideration and preserve such structure as to optimize its immunogenicity.

Yeast vehicles can be formulated into yeast-based immunotherapy compositions or products of the present invention, including preparations to be administered to a subject directly or first loaded into a carrier such as a dendritic cell, using a number of techniques known to those skilled in the art. For example, yeast vehicles can be dried by lyophilization. Formulations comprising yeast vehicles can also be prepared by packing yeast in a cake or a tablet, such as is done for yeast used in baking or brewing operations. In addition, yeast vehicles can be mixed with a pharmaceutically acceptable excipient, such as an isotonic buffer that is tolerated by a host or host cell. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity-enhancing agents, such as sodium carboxymethylcellulose, sorbitol, glycerol or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise, for example, dextrose, human serum albumin, and/or preservatives to which sterile water or saline can be added prior to administration.

In one embodiment of the present invention, a composition can include additional agents and/or biological response modifier compounds, or the ability to produce such modifiers (i.e., by transfection of the yeast vehicle with nucleic acid molecules encoding such modifiers).

Such agents may include, but are not limited to, cytokines, chemokines, hormones, lipidic derivatives, peptides, proteins, polysaccharides, small molecule drugs, antibodies and antigen binding fragments thereof (including, but not limited to, anti-cytokine antibodies, anti-cytokine receptor antibodies, anti-chemokine antibodies), vitamins, polynucleotides, nucleic acid binding moieties, aptamers, and growth modulators. Some suitable agents include, but are not limited to, IL-1 or agonists of IL-1 or of IL-1R, anti-IL-1 or other IL-1 antagonists; IL-6 or agonists of IL-6 or of IL-6R, anti-IL-6 or other IL-6 antagonists; IL-12 or agonists of IL-12 or of IL-12R, anti-IL-12 or other IL-12 antagonists; IL-17 or agonists of IL-17 or of IL-17R, anti-IL-17 or other IL-17 antagonists; IL-21 or agonists of IL-21 or of IL-21R, anti-IL-21 or other IL-21 antagonists; IL-22 or agonists of IL-22 or of IL-22R, anti-IL-22 or other IL-22 antagonists; IL-23 or agonists of IL-23 or of IL-23R, anti-IL-23 or other IL-23 antagonists; IL-25 or agonists of IL-25 or of IL-25R, anti-IL-25 or other IL-25 antagonists; IL-27 or agonists of IL-27 or of IL-27R, anti-IL-27 or other IL-27 antagonists; type I interferon (including IFN-α) or agonists or antagonists of type I interferon or a receptor thereof; type II interferon (including IFN-γ) or agonists or antagonists of type II interferon or a receptor thereof; anti-CD40, CD40L, anti-CTLA-4 antibody (e.g., to release anergic T cells); T cell co-stimulators (e.g., anti-CD137, anti-CD28, anti-CD40); alemtuzumab (e.g., CamPath®), denileukin diftitox (e.g., ONTAK®); anti-CD4; anti-CD25; anti-PD-1, anti-PD-L1, anti-PD-L2; agents that block FOXP3 (e.g., to abrogate the activity/kill CD4+/CD25+ T regulatory cells); Flt3 ligand, imiquimod (Aldara™), granulocyte-macrophage colony stimulating factor (GM-CSF); granulocyte-colony stimulating factor (G-CSF), sargramostim (Leukine®); hormones including without limitation prolactin and growth hormone; Toll-like receptor (TLR) agonists, including but not limited to TLR-2 agonists, TLR-4 agonists, TLR-7 agonists, and TLR-9 agonists; TLR antagonists, including but not limited to TLR-2 antagonists, TLR-4 antagonists, TLR-7 antagonists, and TLR-9 antagonists; anti-inflammatory agents and immunomodulators, including but not limited to, COX-2 inhibitors (e.g., Celecoxib, NSAIDS), glucocorticoids, statins, and thalidomide and analogues thereof including IMiD™s (which are structural and functional analogues of thalidomide (e.g., REVLIMID® (lenalidomide), ACTIMID® (pomalidomide)); proinflammatory agents, such as fungal or bacterial components or any proinflammatory cytokine or chemokine; immunotherapeutic vaccines including, but not limited to, virus-based vaccines, bacteria-based vaccines, or antibody-based vaccines; and any other immunomodulators, immunopotentiators, anti-inflammatory agents, and/or pro-inflammatory agents. Any combination of such agents is contemplated by the invention, and any of such agents combined with or administered in a protocol with (e.g., concurrently, sequentially, or in other formats with) a yeast-based immunotherapeutic is a composition encompassed by the invention. Such agents are well known in the art. These agents may be used alone or in combination with other agents described herein.

Agents of the invention can, in some aspects be referred to as biological response modifier compounds, and the invention includes the ability to produce such modifiers (i.e., by transfection of the yeast vehicle with nucleic acid molecules encoding such modifiers). For example, a yeast vehicle can be transfected with or loaded with at least one antigen and at least one biological response modifier compound, or a composition of the invention can be administered in conjunction with at least one biological response modifier. Biological response modifiers include adjuvants and other compounds that can modulate immune responses, which may be referred to as immunomodulatory compounds, as well as compounds that modify the biological activity of another compound or agent, such as a yeast-based immunotherapeutic, such biological activity not being limited to immune system effects. Certain immunomodulatory compounds can stimulate a protective immune response whereas others can suppress a harmful immune response, and whether an immunomodulatory is useful in combination with a given yeast-based immunotherapeutic may depend, at least in part, on the disease state or condition to be treated or prevented, and/or on the individual who is to be treated. Certain biological response modifiers preferentially enhance a cell-mediated immune response whereas others preferentially enhance a humoral immune response (i.e., can stimulate an immune response in which there is an increased level of cell-mediated compared to humoral immunity, or vice versa.). Certain biological response modifiers have one or more properties in common with the biological properties of yeast-based immunotherapeutics or enhance or complement the biological properties of yeast-based immunotherapeutics. There are a number of techniques known to those skilled in the art to measure stimulation or suppression of immune responses, as well as to differentiate cell-mediated immune responses from humoral immune responses.

Agents can include agonists and antagonists of a given protein or peptide or domain thereof. As used herein, an “agonist” is any compound or agent, including without limitation small molecules, proteins, peptides, antibodies, nucleic acid binding agents, etc., that binds to a receptor or ligand and produces or triggers a response, which may include agents that mimic the action of a naturally occurring substance that binds to the receptor or ligand. An “antagonist” is any compound or agent, including without limitation small molecules, proteins, peptides, antibodies, nucleic acid binding agents, etc., that blocks or inhibits or reduces the action of an agonist.

Compositions of the invention can further include any other compounds that are useful for protecting a subject from a particular infectious disease or any compounds that treat or ameliorate any symptom of such an infection.

Accordingly, the invention also includes a variety of compositions that are useful in the methods of the invention, various aspects of which have been described in detail herein.

The invention also includes a kit comprising any of the compositions described herein, or any of the individual components of the compositions described herein.

### Methods for Administration or Use of Compositions of the Invention

The present invention includes the delivery (administration, immunization) of a composition of the invention to a subject. The administration process can be performed ex vivo or in vivo, but is typically performed in vivo. Ex vivo administration refers to performing part of the regulatory step outside of the patient, such as administering a composition of the present invention to a population of cells (dendritic cells) removed from a patient under conditions such that a yeast vehicle, antigen(s) and any other agents or compositions are loaded into the cell, and returning the cells to the patient. The therapeutic composition of the present invention can be returned to a patient, or administered to a patient, by any suitable mode of administration.

Administration of a composition can be systemic, mucosal and/or proximal to the location of the target site (e.g., near a site of infection). Suitable routes of administration will be apparent to those of skill in the art, depending on the type of condition to be prevented or treated, the antigen used, and/or the target cell population or tissue. Various acceptable methods of administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, aural, intranasal, oral, pulmonary administration, impregnation of a catheter, and direct injection into a tissue. In one aspect, routes of administration include: intravenous, intraperitoneal, subcutaneous, intradermal, intranodal, intramuscular, transdermal, inhaled, intranasal, oral, intraocular, intraarticular, intracranial, and intraspinal. Parenteral delivery can include intradermal, intramuscular, intraperitoneal, intrapleural, intrapulmonary, intravenous, subcutaneous, atrial catheter and venal catheter routes. Aural delivery can include ear drops, intranasal delivery can include nose drops or intranasal injection, and intraocular delivery can include eye drops. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Other routes of administration that modulate mucosal immunity are useful in the treatment of viral infections. Such routes include bronchial, intradermal, intramuscular, intranasal, other inhalatory, rectal, subcutaneous, topical, transdermal, vaginal and urethral routes. In one aspect, a yeast-based immunotherapeutic composition of the invention is administered subcutaneously.

Various methods of the invention treat a disease or condition by administering compositions of the invention. As used herein, the phrase “treat a disease”, or any permutation thereof (e.g., “treated for a disease”, etc.) can generally refer to preventing a disease, preventing at least one symptom of the disease, delaying onset of a disease, reducing one or more symptoms of the disease, reducing the occurrence of the disease, and/or reducing the severity of the disease. With respect to infectious disease and other diseases, the methods of the invention can result in one or more of: prevention of the disease or condition, prevention of infection, delay the onset of disease or symptoms caused by the infection, increased survival, reduction of pathogen burden (e.g., reduction of viral titer), reduction in at least one symptom resulting from the infection in the individual, reduction of organ or physiological system damage resulting from the infection or disease, improvement in organ or system function, and/or improved general health of the individual.

With respect to the yeast-based immunotherapy compositions of the invention, in general, a suitable single dose is a dose that is capable of effectively providing a yeast vehicle and an antigen (if included) to a given cell type, tissue, or region of the patient body in an amount effective to elicit an antigen-specific immune response, when administered one or more times over a suitable time period. For example, in one embodiment, a single dose of a yeast vehicle of the present invention is from about 1×10⁵ to about 5×10⁷ yeast cell equivalents per kilogram body weight of the organism being administered the composition. In one aspect, a single dose of a yeast vehicle of the present invention is from about 0.1 Y.U. (1×10⁶ cells) to about 100 Y.U. (1×10⁹ cells) per dose (i.e., per organism), including any interim dose, in increments of 0.1×10⁶ cells (i.e., 1.1×10⁶, 1.2×10⁶, 1.3×10⁶ . . . ). In one embodiment, doses include doses between 1 Y.U and 40 Y.U. and in one aspect, between 10 Y.U. and 40 Y.U. In one embodiment, the doses are administered at different sites on the individual but during the same dosing period. For example, a 40 Y.U. dose may be administered via by injecting 10 Y.U. doses to four different sites on the individual during one dosing period.

“Boosters” or “boosts” of a therapeutic composition are administered, for example, when the immune response against the antigen has waned or as needed to provide an immune response or induce a memory response against a particular antigen or antigen(s). Boosters can be administered from about 1, 2, 3, 4, 5, 6, 7, or 8 weeks apart, to monthly, to bimonthly, to quarterly, to annually, to several years after the original administration. In one embodiment, an administration schedule is one in which from about 1×10⁵ to about 5×10⁷ yeast cell equivalents of a composition per kg body weight of the organism is administered at least 1, 2, 3, 4, 5, 6, 7, 8, 9, 10, or more times over a time period of from weeks, to months, to years.

In one aspect of the invention, the yeast-based immunotherapy composition. In one aspect of the invention, the agent is administered sequentially with the yeast-based immunotherapy composition. In another embodiment, the agent is administered before the yeast-based immunotherapy composition is administered. In another embodiment, the agent is administered after the yeast-based immunotherapy composition is administered. In one embodiment, the agent is administered in alternating doses with the yeast-based immunotherapy composition, or in a protocol in which the yeast-based composition is administered at prescribed intervals in between or with one or more consecutive doses of the agent, or vice versa. In one embodiment, the yeast-based immunotherapy composition is administered in one or more doses over a period of time prior to commencing the administration of the agent. In other words, the yeast-based immunotherapeutic composition is administered as a monotherapy for a period of time, and then the agent administration is added, either concurrently with new doses of yeast-based immunotherapy, or in an alternating fashion with yeast-based immunotherapy. Alternatively, the agent may be administered for a period of time prior to beginning administration of the yeast-based immunotherapy composition. In one aspect, the yeast is engineered to express or carry the agent, or a different yeast is engineered or produced to express or carry the agent.

In one aspect, the one or more therapies are used in conjunction with immunotherapy, include administering one or both of at least one interferon and at least one anti-viral compound. In this embodiment, the additional therapies, such as the interferon and anti-viral compound, are first administered at least 4 weeks after the immunotherapeutic composition is first administered. In other aspects of this embodiment, the additional therapies such as interferon and anti-viral compound are first administered at least 4 to 12 weeks after the immunotherapeutic composition is first administered, and in another aspect, at least 12 weeks after the immunotherapeutic composition is first administered. Preferably, interferon is administered to the subject weekly for between 24 and 48 weeks, or longer, and over the same period of time, the anti-viral compound is administered daily. In one aspect, the anti-viral compound is ribavirin. In another aspect, the interferon is administered to the subject during concurrent anti-viral therapy every 2, 3 or 4 weeks, for at least 24 weeks, 48 weeks, or longer. In one embodiment, the dosing of anti-viral compound is daily, every 2 days, every 3 days, every 4 days, every 5 days, every 6 days, or weekly, with daily being one preferred embodiment.

As used herein, the term “anti-viral compound” refers to any compound, typically a small-molecule inhibitor or antibody, which targets one or more various steps in the HCV life cycle with direct antiviral therapeutic effects. Anti-viral compounds for HCV treatment are sometimes called “Specifically Targeted Antiviral Therapy for Hepatitis C” or “STAT-C”. Examples of anti-viral compounds include, but are not limited to, viral protease inhibitors (e.g., TELAPREVIR™, an NS3 protease inhibitor from Vertex/Johnson & Johnson/Mitsubishi; BOCEPREVIR™, an NS3 protease inhibitor from Merck & Co., Inc.; RG7227, an inhibitor of the HCV NS3/4 protease, InterMune, Inc./Roche), polymerase inhibitors (e.g., R-7128, a prodrug of an oral cytidine nucleoside analog and an NS5b polymerase inhibitor from Roche/Pharmasset; PSI-7977, a uridine nucleotide analog polymerase inhibitor from Pharmasset; PSI-938, a guanine nucleotide analog polymerase inhibitor from Pharmasset), or other viral inhibitors (e.g., TARIBAVIRIN™ (viramidine) from Valeant). The term “anti-viral compound” as used herein also includes host enzyme inhibitors. Anti-viral compounds for HBV treatment include lamivudine (EPIVIR®), adefovir (HEPSERA®), tenofovir (VIREAD®), telbivudine (TYZEKA®) and entecavir (BARACLUDE®).

Ribavirin is an example of an anti-viral compound useful in the invention, although the invention is not limited to this anti-viral compound. Other anti-viral compounds and their dosing regimens are discussed elsewhere herein and/or are known in the art. “Ribavirin” is an ribosyl purine analogue with an incomplete purine 6-membered ring. The chemical name of ribavirin is 1-(beta)-D-ribofuranosyl-1H-1,2,4-triazole-3-carboxamide. The empirical formula of ribavirin is C₈H₁₂N₄O₅ and the molecular weight is 244.2. Ribavirin is a white to off-white powder. It is freely soluble in water and slightly soluble in anhydrous alcohol. Ribavirin's carboxamide group can make the native nucleoside drug resemble adenosine or guanosine, depending on its rotation. Ribavirin is a prodrug that is activated by cellular kinases, which change it into the 5′ triphosphate nucleotide. In this form, it interferes with aspects of RNA metabolism related to viral replication. Derivatives of ribavirin are well-known in the art and are marketed as (COPEGUS™; REBETOL™; RIBASPHERE™; VILONA™, VIRAZOLE™, also generics from Sandoz, Teva, Warrick). Ribavirin is commercially available in 200 mg tablets or capsules, although any suitable form of dose or delivery type is encompassed by the invention. The dose can be varied according to the preferences and recommendations of the physician, and whether the ribavirin is combined interferon, and it is within the abilities of those of skill in the art to determine the proper dose. A suitable dose of ribavirin, when used in conjunction with interferon, can range from approximately 800 mg to approximately 1200 mg daily, including any increment in between these doses (e.g., 900 mg, 1000 mg, 1100 mg, etc.). Typically, dosing is determined based on body weight, where persons of higher weight take a higher dose of ribavirin. In a preferred embodiment, ribavirin is administered daily at between 1000 mg (subject <75 kg) to 1200 mg (subject ≧75 kg), administered orally in two divided doses. The dose is preferably individualized to the patient depending on baseline weight and tolerability of the regimen (according to product directions).

“Host Enzyme Inhibitors” act indirectly, as they target neither the virus nor the immune system. These molecules work by inhibiting a host cell function exploited by a virus. Examples of such inhibitors include, but are not limited to, cyclophilin B inhibitors, alpha glucosidase inhibitors, PFOR inhibitors, and IRES inhibitors. Exemplary host enzyme inhibitors include, but are not limited to, DEBIO-025™ (Debiopharma), a cyclophilin B inhibitor; CELGOSIVIR™ (Migenix), an oral alpha glucosidase inhibitor; NIM811™ (Novartis), a cyclophilin B inhibitor; ALINIA™ (nitazoxanide, by Romark), a PFOR inhibitor; and VGX-410C™ (VGX Pharma), an oral IRES inhibitor.

BOCEPREVIR® is an NS3 protease inhibitor. For the treatment of HCV, the drug is currently administered orally at a dose of 800 mg three times a day.

TELAPREVIR® is an NS3 protease inhibitor. For the treatment of HCV, the drug is currently administered orally at a dose of 750 mg.

“Lamivudine”, or 2′,3′-dideoxy-3′-thiacytidine, commonly called 3TC, is a potent nucleoside analog reverse transcriptase inhibitor (nRTI). For the treatment of HBV infection, lamivudine is administered as a pill or oral solution taken at a dose of 100 mg once a day (1.4-2 mg/lb. twice a day for children 3 months to 12 years old).

“Adefovir” (adefovir dipivoxil), or 9-[2-[[bis[(pivaloyloxy)methoxy]-phosphinyl]-methoxy]ethyl]adenine, is an orally-administered nucleotide analog reverse transcriptase inhibitor (ntRTI). For the treatment of HBV infection, adefovir is administered as a pill taken at a dose of 10 mg once daily.

“Tenofovir” (tenofovir disoproxil fumarate), or ({[(2R)-1-(6-amino-9H-purin-9-yl)propan-2-yl]oxy}methyl)phosphonic acid, is a nucleotide analogue reverse transcriptase inhibitor (nRTIs). For the treatment of HBV, tenofovir is administered as a pill taken at a dose of 300 mg (tenofovir disproxil fumarate) once daily.

“Telbivudine”, or 1-(2-deoxy-β-L-erythro-pentofuranosyl)-5-methylpyrimidine-2,4(1H,3H)-dione, is a synthetic thymidine nucleoside analogue (the L-isomer of thymidine). For the treatment of HBV infection, telbivudine is administered as a pill or oral solution taken at a dose of 600 mg once daily.

“Entecavir”, or 2-Amino-9-[(1S,3R,4S)-4-hydroxy-3-(hydroxymethyl)-2-methylidenecyclopentyl]-6,9-dihydro-3H-purin-6-one, is a nucleoside analog (guanine analogue) that inhibits reverse transcription, DNA replication and transcription of the virus. For the treatment of HBV infection, entecavir is administered as a pill or oral solution taken at a dose of 0.5 mg once daily (1 mg daily for lamivudine-refractory or telbivudine resistance mutations).

In any of the embodiments of the invention that include the administration of interferon, the interferon can be any interferon. As used herein, the term “interferon” refers to a cytokine that is typically produced by cells of the immune system and by a wide variety of cells in response to the presence of double-stranded RNA. Interferons assist the immune response by inhibiting viral replication within host cells, activating natural killer cells and macrophages, increasing antigen presentation to lymphocytes, and inducing the resistance of host cells to viral infection. Type I interferons include interferon-α. Interferons useful in the methods of the present invention include any type I interferon, and preferably interferon-α, and more preferably, interferon-α2, and more preferably, longer lasting forms of interferon, including, but not limited to, pegylated interferons, interferon fusion proteins (interferon fused to albumin), and controlled-release formulations comprising interferon (e.g., interferon in microspheres or interferon with polyaminoacid nanoparticles).

In one embodiment of the invention, the interferon is not interferon-α. In one embodiment, the interferon is an interferon-α. In one embodiment, the interferon is not an interferon-λ. In one embodiment, the interferon is an interferon-λ. In one embodiment, the interferon is a “consensus interferon”, or CIFN, which is a new, non-natural type I interferon, approved by the Federal Drug Administration for treatment of chronic hepatitis C virus infection. CIFN was bioengineered to be composed of the most frequently observed amino acid in each corresponding position in the natural alpha interferons (see, e.g., Melian and Plosker, Drugs, 2001, 61(11):1661-91).

One interferon, PEGASYS®, peginterferon α-2a, is a covalent conjugate of recombinant α-2a interferon (approximate molecular weight [MW] 20,000 daltons) with a single branched bis-monomethoxy polyethylene glycol (PEG) chain (approximate MW 40,000 daltons). The PEG moiety is linked at a single site to the interferon a moiety via a stable amide bond to lysine. Peginterferon α-2a has an approximate molecular weight of 60,000 daltons. Interferon-α-2a is produced using recombinant DNA technology in which a cloned human leukocyte interferon gene is inserted into and expressed in Escherichia coli. In one embodiment, the interferon is interferon-α, including, but not limited to interferon-α2 or pegylated interferon-α2.

Another interferon, PEGINTRON®, pegylated interferon-α2b, is a covalent conjugate of recombinant α-2b interferon with monomethoxy polyethylene glycol (PEG) (approximate MW 12,000 daltons).

Interferon is typically administered by intramuscular or subcutaneous injection, and can be administered in a dose of between 3 and 10 million units, with 3 million units being preferred in one embodiment. In another embodiment, the recommended dose of interferon when used in combination with ribavirin for chronic hepatitis C is 180 μg (1.0 mL vial or 0.5 mL prefilled syringe) once weekly (e.g., for PEGASYS®).

Doses of interferon are administered on a regular schedule, which can vary from 1, 2, 3, 4, 5, or 6 times a week, to weekly, biweekly, every three weeks, or monthly. A typical dose of interferon that is currently available is provided weekly, and that is a preferred dosing schedule for interferon, according to the present invention. The dose amount and timing can be varied according to the preferences and recommendations of the physician, as well as according to the recommendations for the particular interferon being used, and it is within the abilities of those of skill in the art to determine the proper dose.

Preferably, when the course of interferon and anti-viral compound therapy begins, additional doses of the immunotherapeutic composition are administered over the same period of time, or for at least a portion of that time, and may continue to be administered once the course of interferon and anti-viral compound has ended. However, the dosing schedule for the immunotherapy over the entire period may be, and is preferably, different than that for the interferon and/or anti-viral compound. For example, the immunotherapeutic composition may be administered on the same days or at least 3-4 days after the last given (most recent) dose of interferon (or any suitable number of days after the last dose), and may be administered weekly, biweekly, monthly, bimonthly, or every 3-6 months. During the initial period of monotherapy administration of the immunotherapeutic composition, the composition is preferably administered weekly for between 4 and 12 weeks, followed by monthly administration (regardless of when the additional interferon/anti-viral therapy is added into the protocol). In one aspect, the immunotherapeutic composition is administered weekly for five weeks, followed by monthly administration thereafter, until conclusion of the complete treatment protocol.

In aspects of the invention, a yeast-based immunotherapeutic composition and other agents can be administered together (concurrently). As used herein, concurrent use does not necessarily mean that all doses of all compounds are administered on the same day at the same time. Rather, concurrent use means that each of the therapy components (e.g., immunotherapy and interferon therapy, and the anti-viral therapy, if added) are started at approximately the same period (within hours, or up to 1-7 days of each other) and are administered over the same general period of time, noting that each component may have a different dosing schedule (e.g., interferon weekly and immunotherapy monthly, with addition of daily doses of ribavirin, etc.). In addition, before or after the concurrent administration period, any one of the agents or immunotherapeutic compositions can be administered without the other agent(s).

In the method of the present invention, compositions and therapeutic compositions can be administered to animal, including any vertebrate, and particularly to any member of the Vertebrate class, Mammalia, including, without limitation, primates, rodents, livestock and domestic pets. Livestock include mammals to be consumed or that produce useful products (e.g., sheep for wool production). Mammals to protect include humans, dogs, cats, mice, rats, goats, sheep, cattle, horses and pigs.

An “individual” is a vertebrate, such as a mammal, including without limitation a human. Mammals include, but are not limited to, farm animals, sport animals, pets, primates, mice and rats. The term “individual” can be used interchangeably with the term “animal”, “subject” or “patient”.

### General Techniques Useful in the Invention

The practice of the present invention will employ, unless otherwise indicated, conventional techniques of molecular biology (including recombinant techniques), microbiology, cell biology, biochemistry, nucleic acid chemistry, and immunology, which are well known to those skilled in the art. Such techniques are explained fully in the literature, such as, Methods of Enzymology, Vol. 194, Guthrie et al., eds., Cold Spring Harbor Laboratory Press (1990); Biology and activities of yeasts, Skinner, et al., eds., Academic Press (1980); Methods in yeast genetics: a laboratory course manual, Rose et al., Cold Spring Harbor Laboratory Press (1990); The Yeast Saccharomyces: Cell Cycle and Cell Biology, Pringle et al., eds., Cold Spring Harbor Laboratory Press (1997); The Yeast Saccharomyces: Gene Expression, Jones et al., eds., Cold Spring Harbor Laboratory Press (1993); The Yeast Saccharomyces: Genome Dynamics, Protein Synthesis, and Energetics, Broach et al., eds., Cold Spring Harbor Laboratory Press (1992); Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y. (jointly referred to herein as “Harlow and Lane”), Beaucage et al. eds., Current Protocols in Nucleic Acid Chemistry John Wiley & Sons, Inc., New York, 2000); Casarett and Doull's Toxicology The Basic Science of Poisons, C. Klaassen, ed., 6th edition (2001), and Vaccines, S. Plotkin and W. Orenstein, eds., 3rd edition (1999).

### GENERAL DEFINITIONS

“Standard Of Care (SOC)” generally refers to the current approved standard of care for the treatment of a specific infectious disease, or a diagnostic or treatment process that a clinician should follow for a certain type of patient, illness, or clinical circumstance. In some diseases, such as in chronic HBV infection, SOC may include one of several different approved therapeutic protocols which include various anti-viral drugs (lamivudine (EPIVIR®), adefovir (HEPSERA®), tenofovir (VIREAD®), telbivudine (TYZEKA®) and entecavir (BARACLUDE®)) or type I interferon (e.g., pegylated interferon-α). With respect to HCV infection, SOC refers to the current standard of care for the treatment of hepatitis C virus, which consists essentially of the administration of a combination of interferon (preferably interferon-α2, and more preferably, pegylated interferon-α) with the anti-viral compound, ribavirin. The combination is typically administered by subcutaneous injection of interferon once weekly for 24 weeks (HCV genotypes 2 and 3) or 48 weeks (HCV genotypes 1 and 4), with concurrent administration of ribavirin, typically administered orally on a daily dosing schedule. New protocols for HCV therapy which may ultimately be considered to be SOC for HCV include the combination of current standard of care (pegylated interferon-α and ribavirin), with one of two protease inhibitors known as TELAPREVIR™, an NS3 protease inhibitor from Vertex/Johnson & Johnson/Mitsubishi or BOCEPREVIR™, an NS3 protease inhibitor from Merck & Co., Inc. In addition, other anti-virals are in development, including a polymerase inhibitor PSI-7977, a uridine nucleotide analog polymerase inhibitor from Pharmasset, which is used in combination with pegylated interferon-α and ribavirin, or the combination of PSI-7977 and PSI-938, a guanine nucleotide analog polymerase inhibitor from Pharmasset, which are used together in the absence of interferon.

“Viral negativity” or “complete response”, which terms may be capitalized, can be used interchangeably herein and are defined for HCV as HCV RNA <25 IU/ml, which includes undetectable virus. A “complete responder” is a subject who has achieved a complete response. For HBV, viral negativity is typically defined as below detectable levels by PCR or <2000 IU/ml.

“Rapid Virologic Response (RVR)” for HCV is defined as viral negativity after 4 weeks of interferon-based therapy.

“Early Virologic Response (EVR)” for HCV is defined as >2 log 10 reduction in viral load by week 12 of interferon-based therapy.

“Complete EVR (cEVR)” for HCV is defined as viral negativity by week 12 of interferon-based therapy.

“End of Treatment Response (ETR)” for HCV is defined as viral negativity by 48 weeks of interferon-based therapy for interferon-naïve subjects, and as viral negativity by 72 weeks for Non-Responders (for genotype 1 patients).

“Sustained Virologic Response (SVR or SVR24)” for HCV is defined as viral negativity at 6 months post ETR.

“Naïve” or “Interferon-naïve” subjects (patients) for HCV are subjects who have not been previously treated with interferon or SOC (interferon plus ribavirin).

“Null Responders” are HCV infected subjects that cannot achieve at least a 1 log 10 reduction in viral load by week 12 on SOC.

“Non-Responders” are subjects who have received a 12-week course of interferon-based therapy and failed to achieve EVR.

“Partial Responders” are defined as subjects who have >2 log 10 viral load reduction by 12 weeks, but never achieve viral negativity.

“Poor Responders” are defined as subjects who have between 1-2 logᵢₙ viral load reduction by 12 weeks, but never achieve viral negativity.

“Breakthrough” subjects or “Treatment-breakthrough” subjects are subjects who achieve viral negativity during treatment, but whose viral loads return to detectable levels before end of treatment (ETR endpoint).

“Relapsers” are subjects who achieve viral eradication (negativity) by end of treatment (ETR endpoint), but whose viral load returns to detectable levels during the 24 week follow up.

“Seroconversion” in HBV patients refers to HBeAg/HBsAg seroconversion, which is loss of HBeAg and HBsAg and the development of antibodies against the hepatitis B surface antigen (anti-HBs) and/or antibodies against HBeAg. Clinical studies have defined seroconversion, or a protective antibody (anti-HBs) level as: (a) 10 or more sample ratio units (SRU) as determined by radioimmunoassay; (b) a positive result as determined by enzyme immunoassay; or (c) detection of an antibody concentration of >10 mIU/ml (10 SRU is comparable to 10 mIU/mL of antibody).

“ALT” is a well-validated measure of hepatic injury and serves as a surrogate for hepatic inflammation. In prior large hepatitis trials, reductions and/or normalization of ALT levels (ALT normalization) have been shown to correlate with improved liver function and reduced liver fibrosis as determined by serial biopsy.

An “immunotherapeutic composition” is a composition that elicits an immune response sufficient to achieve at least one therapeutic benefit in a subject.

A “TARMOGEN®” (GlobeImmune, Inc., Louisville, Colo.) is an example of a yeast-based immunotherapeutic and generally refers to a yeast vehicle expressing one or more heterologous antigens extracellularly (on its surface), intracellularly (internally or cytosolically) or both extracellularly and intracellularly. TARMOGEN® products have been generally described (see, e.g., U.S. Pat. No. 5,830,463). Certain yeast-based immunotherapy compositions, and methods of making and generally using the same, are also described in detail, for example, in U.S. Pat. No. 5,830,463, U.S. Pat. No. 7,083,787, U.S. Pat. No. 7,736,642, Stubbs et al., Nat. Med. 7:625-629 (2001), Lu et al., Cancer Research 64:5084-5088 (2004), and in Bernstein et al., Vaccine 2008 Jan. 24; 26(4):509-21, each of which is incorporated herein by reference in its entirety.

As used herein, the term “analog” refers to a chemical compound that is structurally similar to another compound but differs slightly in composition (as in the replacement of one atom by an atom of a different element or in the presence of a particular functional group, or the replacement of one functional group by another functional group). Thus, an analog is a compound that is similar or comparable in function and appearance, but has a different structure or origin with respect to the reference compound.

The terms “substituted”, “substituted derivative” and “derivative”, when used to describe a compound, means that at least one hydrogen bound to the unsubstituted compound is replaced with a different atom or a chemical moiety.

Although a derivative has a similar physical structure to the parent compound, the derivative may have different chemical and/or biological properties than the parent compound. Such properties can include, but are not limited to, increased or decreased activity of the parent compound, new activity as compared to the parent compound, enhanced or decreased bioavailability, enhanced or decreased efficacy, enhanced or decreased stability in vitro and/or in vivo, and/or enhanced or decreased absorption properties.

In general, the term “biologically active” indicates that a compound has at least one detectable activity that has an effect on the metabolic or other processes of a cell or organism, as measured or observed in vivo (i.e., in a natural physiological environment) or in vitro (i.e., under laboratory conditions).

According to the present invention, the general use herein of the term “antigen” refers: to any portion of a protein (peptide, partial protein, full-length protein), wherein the protein is naturally occurring or synthetically derived, to a cellular composition (whole cell, cell lysate or disrupted cells), to an organism (whole organism, lysate or disrupted cells) or to a carbohydrate, or other molecule, or a portion thereof. An antigen may elicit an antigen-specific immune response (e.g., a humoral and/or a cell-mediated immune response) against the same or similar antigens that are encountered by an element of the immune system (e.g., T cells, antibodies).

An antigen can be as small as a single epitope, or larger, and can include multiple epitopes. As such, the size of an antigen can be as small as about 5-12 amino acids (e.g., a peptide) and as large as: a full length protein, including a multimer and fusion proteins, chimeric proteins, whole cells, whole microorganisms, or portions thereof (e.g., lysates of whole cells or extracts of microorganisms). In addition, antigens can include carbohydrates, which can be loaded into a yeast vehicle or into a composition of the invention. It will be appreciated that in some embodiments (i.e., when the antigen is expressed by the yeast vehicle from a recombinant nucleic acid molecule), the antigen is a protein, fusion protein, chimeric protein, or fragment thereof, rather than an entire cell or microorganism.

When referring to stimulation of an immune response, the term “immunogen” is a subset of the term “antigen”, and therefore, in some instances, can be used interchangeably with the term “antigen”. An immunogen, as used herein, describes an antigen which elicits a humoral and/or cell-mediated immune response (i.e., is immunogenic), such that administration of the immunogen to an individual mounts an antigen-specific immune response against the same or similar antigens that are encountered by the immune system of the individual.

An “immunogenic domain” of a given antigen can be any portion, fragment or epitope of an antigen (e.g., a peptide fragment or subunit or an antibody epitope or other conformational epitope) that contains at least one epitope that acts as an immunogen when administered to an animal. For example, a single protein can contain multiple different immunogenic domains. Immunogenic domains need not be linear sequences within a protein, such as in the case of a humoral immune response.

An epitope is defined herein as a single immunogenic site within a given antigen that is sufficient to elicit an immune response. Those of skill in the art will recognize that T cell epitopes are different in size and composition from B cell epitopes, and that epitopes presented through the Class I MHC pathway differ from epitopes presented through the Class II MHC pathway. Epitopes can be linear sequence or conformational epitopes (conserved binding regions).

An “individual” or a “subject” or a “patient”, which terms may be used interchangeably, is a vertebrate, preferably a mammal, more preferably a human. Mammals include, but are not limited to, farm animals, sport animals, pets, primates, mice and rats.

According to the present invention, the term “modulate” can be used interchangeably with “regulate” and refers generally to upregulation or downregulation of a particular activity. As used herein, the term “upregulate” can be used generally to describe any of: elicitation, initiation, increasing, augmenting, boosting, improving, enhancing, amplifying, promoting, or providing, with respect to a particular activity. Similarly, the term “downregulate” can be used generally to describe any of: decreasing, reducing, inhibiting, ameliorating, diminishing, lessening, blocking, or preventing, with respect to a particular activity.

According to the present invention, “heterologous amino acids” are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived.

According to the present invention, reference to a “heterologous” protein or “heterologous” antigen, including a heterologous fusion protein, in connection with a yeast vehicle of the invention means that the protein or antigen is not a protein or antigen that is naturally expressed by the yeast, although a fusion protein may include yeast sequences or proteins or portions thereof that are naturally expressed by yeast.

In one embodiment of the present invention, any of the amino acid sequences described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. As discussed above, according to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

According to the present invention, the phrase “selectively binds to” refers to the ability of an antibody, antigen-binding fragment or binding partner of the present invention to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen-binding fragment thereof in the absence of the antigen is considered to be background. Binding can be measured using a variety of methods standard in the art including enzyme immunoassays (e.g., ELISA), immunoblot assays, etc.).

Reference to an isolated protein or polypeptide in the present invention includes full-length proteins, fusion proteins, or any fragment, domain, conformational epitope, or homologue of such proteins. More specifically, an isolated protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein of the present invention is produced recombinantly. According to the present invention, the terms “modification” and “mutation” can be used interchangeably, particularly with regard to the modifications/mutations to the amino acid sequence of proteins or portions thereof (or nucleic acid sequences) described herein.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein. Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

A homologue of a given protein including any protein or immunogenic domain described herein, may comprise, consist essentially of, or consist of, an amino acid sequence that is at least about 45%, or at least about 50%, or at least about 55%, or at least about 60%, or at least about 65%, or at least about 70%, or at least about 75%, or at least about 80%, or at least about 85%, or at least about 90%, or at least about 95% identical, or at least about 95% identical, or at least about 96% identical, or at least about 97% identical, or at least about 98% identical, or at least about 99% identical (or any percent identity between 45% and 99%, in whole integer increments), to the amino acid sequence of the reference protein. In one embodiment, the homologue comprises, consists essentially of, or consists of, an amino acid sequence that is less than 100% identical, less than about 99% identical, less than about 98% identical, less than about 97% identical, less than about 96% identical, less than about 95% identical, and so on, in increments of 1%, to less than about 70% identical to the naturally occurring amino acid sequence of the reference protein.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

An isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene. An isolated nucleic acid molecule that includes a gene is not a fragment of a chromosome that includes such gene, but rather includes the coding region and regulatory regions associated with the gene, but no additional genes that are naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein or domain of a protein.

A recombinant nucleic acid molecule is a molecule that can include at least one of any nucleic acid sequence encoding any one or more proteins described herein operatively linked to at least one of any transcription control sequence capable of effectively regulating expression of the nucleic acid molecule(s) in the cell to be transfected. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. In addition, the phrase “recombinant molecule” primarily refers to a nucleic acid molecule operatively linked to a transcription control sequence, but can be used interchangeably with the phrase “nucleic acid molecule” which is administered to an animal.

A recombinant nucleic acid molecule includes a recombinant vector, which is any nucleic acid sequence, typically a heterologous sequence, which is operatively linked to the isolated nucleic acid molecule encoding a fusion protein of the present invention, which is capable of enabling recombinant production of the fusion protein, and which is capable of delivering the nucleic acid molecule into a host cell according to the present invention. Such a vector can contain nucleic acid sequences that are not naturally found adjacent to the isolated nucleic acid molecules to be inserted into the vector. The vector can be either RNA or DNA, either prokaryotic or eukaryotic, and preferably in the present invention, is a virus or a plasmid. Recombinant vectors can be used in the cloning, sequencing, and/or otherwise manipulating of nucleic acid molecules, and can be used in delivery of such molecules (e.g., as in a DNA composition or a viral vector-based composition). Recombinant vectors are preferably used in the expression of nucleic acid molecules, and can also be referred to as expression vectors. Preferred recombinant vectors are capable of being expressed in a transfected host cell.

In a recombinant molecule of the present invention, nucleic acid molecules are operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include nucleic acid molecules that are operatively linked to one or more expression control sequences. The phrase “operatively linked” refers to linking a nucleic acid molecule to an expression control sequence in a manner such that the molecule is expressed when transfected (i.e., transformed, transduced or transfected) into a host cell.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells, such as algae, bacteria and yeast. In microbial systems, the term “transformation” is used to describe an inherited change due to the acquisition of exogenous nucleic acids by the microorganism and is essentially synonymous with the term “transfection.” Therefore, transfection techniques include, but are not limited to, transformation, chemical treatment of cells, particle bombardment, electroporation, microinjection, lipofection, adsorption, infection and protoplast fusion.

The following experimental results are provided for purposes of illustration and are not intended to limit the scope of the invention.

### EXAMPLES

### Example 1

The following example describes the sustained virologic response (SVR) endpoint analysis of a phase 2 trial of subjects treated with GI-5005 immunotherapy in combination with interferon/ribavirin therapy.

GI-5005 is a whole heat-killed Saccharomyces cerevisiae expressing high levels of HCV NS3 and Core antigens. The amino acid sequence of the fusion protein expressed in GI-5005 is represented herein by SEQ ID NO:2. GI-5005 has been designed to elicit antigen-specific host CD4 and CD8 T-cell responses with the goal of improving the rate of immune clearance of HCV, particularly through the immune-mediated elimination of HCV-infected hepatocytes. The GI-5005-02 phase 2 study evaluates the efficacy and safety of GI-5005 plus peg-IFN/ribavirin (SOC) in subjects with genotype 1 chronic HCV infection.

FIG. 1 shows the schematic design of the phase 2 study of GI-5005 (GI-5005-02) in combination with SOC (triple therapy). Genotype 1 subjects with chronic HCV infection who were treatment naïve or non-responders to prior interferon (IFN) or peginterferon (pegIFN) based therapy were eligible (prior null responders and relapsers were excluded). Patients (140 total enrolled) were randomized 1:1, and stratified by virologic response during their prior course of treatment in this open label trial. In Arm 1, GI-5005 was initially administered as a monotherapy run-in consisting of five weekly followed by 2 monthly subcutaneous (SC) doses of 40 YU (1 YU=10,000,000 yeast) GI-5005 over 12 weeks (administered as 10 YU doses to four separate sites on the patient), followed by triple therapy consisting of monthly 40 YU GI-5005 doses plus pegylated interferon (pegIFN) and ribavirin (triple therapy treatment period is 48 weeks in naïve patients and 72 weeks in prior non-responders). Arm 2 patients received treatment with SOC alone (without GI-5005) for the same time periods (48 weeks for naïve or 72 weeks for prior non-responders), and did not receive antecedent GI-5005 monotherapy.

PEGASYS®, or pegylated interferon-α2a, is a covalent conjugate of recombinant α-2a interferon (approximate molecular weight [MW] 20,000 daltons) with a single branched bis-monomethoxy polyethylene glycol (PEG) chain (approximate MW 40,000 daltons). The PEG moiety is linked at a single site to the interferon-α moiety via a stable amide bond to lysine. Pegylated interferon-α2a has an approximate molecular weight of 60,000 daltons. Interferon-α2a is produced using recombinant DNA technology in which a cloned human leukocyte interferon gene is inserted into and expressed in Escherichia coli.

The chemical name of ribavirin is 1-(beta)-D-ribofuranosyl-1H-1,2,4-triazole-3-carboxamide. The empirical formula of ribavirin is C₈H₁₂N₄O₅ and the molecular weight is 244.2. Ribavirin is a white to off-white powder. It is freely soluble in water and slightly soluble in anhydrous alcohol. Ribavirin is a synthetic nucleoside analogue. The mechanism by which the combination of ribavirin and an interferon product exerts its effects against the hepatitis C virus has not been fully established.

Ribavirin and interferon (pegylated IFN-α2a) were administered according to the following recommended dosing information. The recommended dose of PEGASYS® when used in combination with ribavirin for chronic hepatitis C is 180 μg (1.0 mL vial or 0.5 mL prefilled syringe) once weekly. The daily dose of ribavirin is 1000 mg (subject <75 kg) to 1200 mg (subject ≧75 kg) administered orally in two divided doses. The dose should be individualized to the patient depending on baseline weight and tolerability of the regimen.

The study was conducted in 40 centers in the United States, India and Europe. 74% of the total enrolled patients were naïve to prior interferon-based therapy (referred to herein as “interferon-naïve” or just “naïve” individuals); 26% were prior treatment failures (referred to herein as “non-responders”).

All patients in Arm 1 completed GI-5005 triple therapy, all patients in Arm 2 completed SOC therapy, and all naïve and non-responder patients have completed 24 weeks of post-treatment follow up (i.e., all patients have reached the SVR endpoint). ETR*, SVR**, and ALT normalization*** are described in Table 1 below.

TABLE 1

| Endpoints | Triple | SOC | p-value(ITT)# |
|---|---|---|---|
| ETR* all (nₜ = 68, n_{soc} = 65) | 63% | 45% | 0.024 [symbol] |
| ETR naives (nₜ = 50, n_{soc} = 46) | 74% | 59% | 0.085¹ |
| ETR NRs (nₜ = 18, n_{soc} = 19) | 33% | 11% | 0.099¹ |
| SVR** all (nₜ = 68, n_{soc} = 65) | 47% | 35% | 0.117¹ |
| SVR naives (nₜ = 50, n_{soc} = 46) | 58% | 48% | 0.214¹ |
| Relapse (nₜ = 36, n_{soc} = 26) | 19% | 15% | ND |
| missing censored | | | |
| On-treatment breakthrough | 8% | 11% | ND |
| (nₜ = 36, n_{soc} = 28) | | | |
| SVR naives IL28 T/T (nₜ = 5, n_{soc} = 5) | 60% | 0% | 0.166 |
| SVR NRs (nₜ = 18, n_{soc} = 19) | 17% | 5% | 0.214¹ |
| ALT normalization at EoT *** all | 61% | 36% | 0.018² |
| (nₜ = 61 , n_{soc} = 44) | | | |

*ETR = % patients HCV RNA negative by PCR at end of treatment,
**SVR = % with HCV RNA < 25 IU/mL 24 weeks after completion of therapy;
*** % patients with ALT > ULN at baseline (Day 1 of Run-In for Arm 1 and Day 1 of SOC for Arm 2) and at least 2 consecutive visits < ULN,
#Fisher's 1-sided analysis¹ or 2-sided analysis² for ITT includes all patients who received at least 1 dose of triple therapy or SOC. [symbol]
indicates data missing or illegible when filed

The results showed that triple therapy (GI-5005 plus SOC) was well tolerated with no significant new toxicities, related serious adverse events, or growth factor use for anemia or neutropenia observed, and an equivalent number of SOC discontinuations due to adverse events in each group: triple therapy=9/68 (13.2%) and SOC=8/65 (12.3%). At ETR, a statistically significant (p≦0.05) improvement in end of treatment response (ETR) was observed in the group of all patients receiving triple therapy as compared to SOC alone (triple therapy 43/68 [63%] vs. SOC 28/65 [45%]). 47% of all patients receiving triple therapy (32/68) achieved SVR as compared to 35% of all patients receiving SOC alone (23/65); see also FIG. 5.

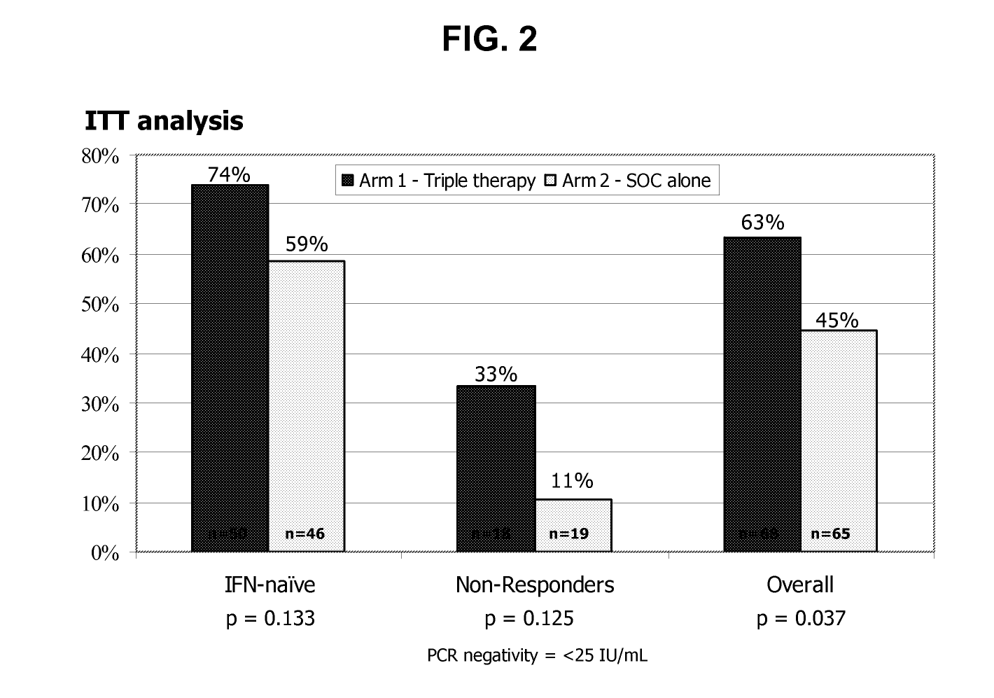

As a group, and as illustrated in FIGS. 2 and 5, naïve patients receiving triple therapy showed a trend toward improvement in ETR over Naïve patients receiving SOC alone (triple therapy 37/50 [74%] vs. SOC 27/46 [59%]), representing a 12% advantage of triple therapy in this study. 58% of Naïve patients receiving triple therapy (29/50) achieved SVR as compared to 48% of naïve patients receiving SOC alone (22/46), representing a 10% advantage of triple therapy. SVRs occurred in both the IL-28B C/C subgroup and notably, in the IL28B T/T subgroup receiving triple therapy (no SVRs occurred in the IL28B T/T subgroup that received SOC alone).

As shown in FIG. 5, a trend toward improvement in end of treatment response (ETR) (triple therapy 6/18 [33%] vs SOC 2/19 [11%]) and SVR (Triple 3/18 [17%] vs SOC 1/19 [5%]) was observed in Non-Responder patients. Due to the small number of Non-Responder patients in each treatment arm, none of these differences were statistically significant (see table). SVR in Non-Responders occurred only in IL28B C/T subjects (Triple 3/11[28%] vs SOC 1/12 [8%]).

In summary, GI-5005 triple therapy delivered improved ETR and SVR (A ranging from 10-22%) in all patients, as well as the treatment-Naïve and Non-Responder subgroups, compared to SOC alone (see table).

Results of the analysis are illustrated in FIGS. 2, 3, 4 and 5. FIG. 2 is a bar graph showing the ITT (Intent To Treat) analysis for End of Treatment (ETR) responses in the phase 2 clinical trial for all patients (Overall), interferon-naïve patients (IFN-naïve), and patients who were previously non-responsive to interferon therapy (Non-Responders), including p-values determined by 1-sided or 2-sided Fisher's exact test, demonstrating that triple therapy (black bars) improved ETR as compared to SOC alone (light bars).

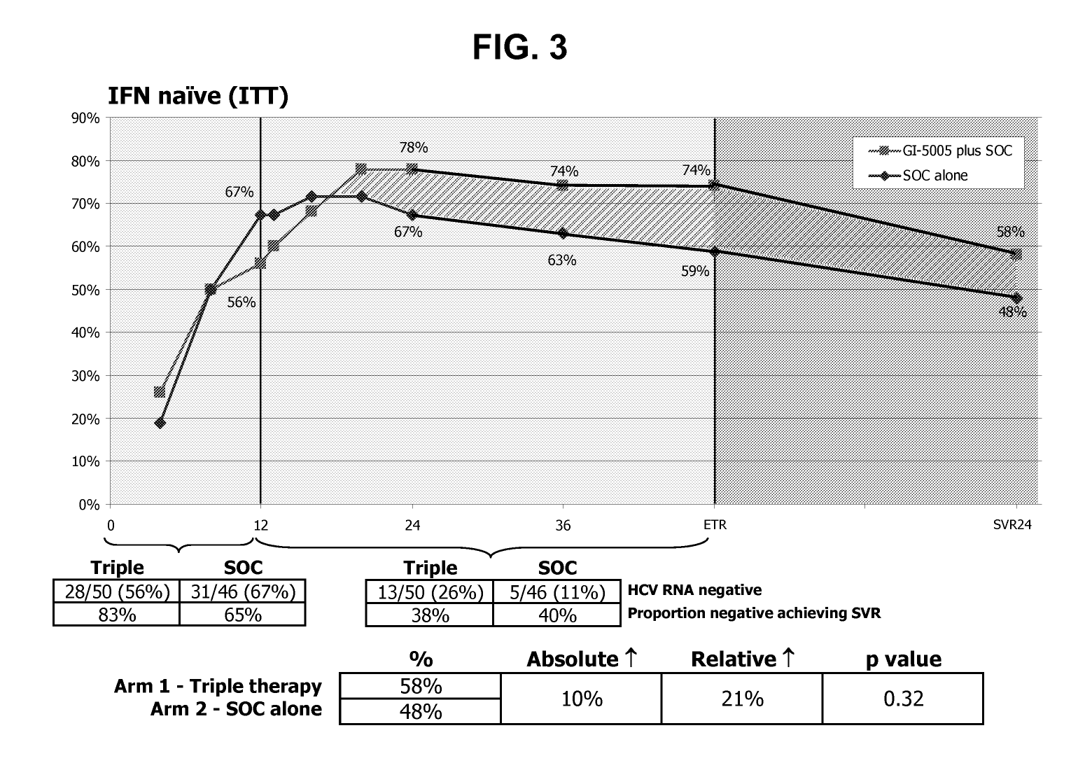

FIG. 3 is a graph showing response kinetics for interferon-naïve subjects receiving triple therapy versus SOC alone, demonstrating that interferon-naïve subjects receiving triple therapy (squares) showed a 10% absolute improvement in SVR (Sustained Virologic Response) and a 21% relative improvement in SVR over interferon-naïve subjects receiving SOC alone (diamonds). FIG. 3 also shows that more subjects receiving triple therapy and achieving viral negativity during the first 12 weeks of treatment (RVR) went on to achieve SVR than subjects receiving SOC alone and achieving viral negativity during the first 12 weeks of treatment.

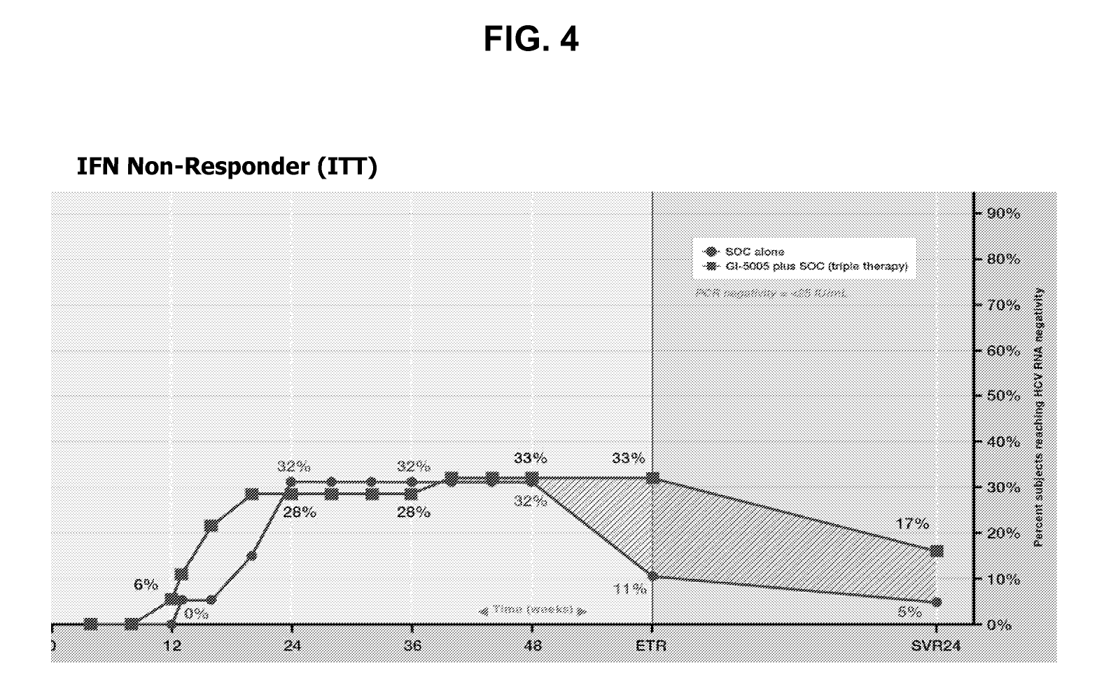

FIG. 4 is a graph showing response kinetics for non-responder subjects receiving triple therapy versus SOC alone, demonstrating that non-responder subjects receiving triple therapy (squares) showed a 12% absolute improvement in SVR (Sustained Virologic Response) over non-responder subjects receiving SOC alone (circles).

FIG. 5 shows the cumulative ETR and SVR data for all patients (Overall), interferon-naïve patients (IFN-naïve), and patients who were previously non-responsive to interferon therapy (Non-Responders), showing that triple therapy improved ETR and SVR in all groups as compared to SOC alone.

In summary, triple therapy significantly improved ETR and ALT normalization (see Example 3) compared to SOC alone. An improvement over SOC alone of 10% in SVR was observed in naïve patients 24 weeks after the completion of therapy, and an improvement over SOC alone of 12% in SVR was observed in non-responder patients 24 weeks after completion of therapy. These data indicate that GI-5005 in combination with SOC, as well as GI-5005 used in novel combinations (e.g., with direct-acting antiviral agents) is a highly effective treatment for chronic HCV infection, and is expected to be effective for treatment of other hepatitis infection (e.g., HBV).

### Example 2

The following example demonstrates that IL28B genotype influences how individuals and/or particular groups of individuals respond to immunotherapy, and also demonstrates that immunotherapy can alter a response of therapy for infectious disease.

IL28B genotypes (C/C, C/T, T/T) predict sustained virologic response (SVR) to standard of care (SOC; PegIFN/ribavirin) and spontaneous clearance of acute HCV (see Ge et al., supra; and Thomas et al., supra). Since GI-5005 generates HCV-specific T-cells involved in spontaneous HCV clearance, the experiment described in this example assessed the influence of IL28B on end of treatment responses (ETR) and SVR responses to GI-5005 plus SOC in naïve and non-responder genotype-1 chronic HCV.

The IL28B locus from all patients was PCR amplified from patient genomic DNA and genotyped by bi-directional sequencing. Briefly, a region encompassing a SNP upstream of the human IL28B gene (rs12979860) was amplified by PCR from genomic DNA isolated from peripheral blood mononuclear cells (PBMCs) or by semi-nested PCR directly from dried blood spots. Samples were assigned randomized numbers and blinded to personnel before testing. PCR primers were as follows: Sense: 5′-TATGTCAGCGCCCACAATTC-3′ (SEQ ID NO:21) and antisense: 5′-GGCTCAGGGTCAATCACAGA-3′ (SEQ ID NO:22) and: 5′-GGAAGGAGCAGTTGCGCTGC-3′ (SEQ ID NO:23).

Genomic DNA (100 ng) was added to a PCR mix consisting of 0.2 mM of dNTPs, 1× high fidelity (HF) PCR buffer (containing 1.5 mM MgCl2, NEB), 0.4 μM of sense and anti-sense primers and 1 unit of thermostable polymerase (Phusion® taq, NEB) in a total volume of 50 μL. PCR was run with a touchdown program as follows: segment i) 98° C. for 2 min; segment ii) 20 cycles of [98° C. for 10 sec, followed by touch down annealing from 64° C. for 30 sec followed by extension at 72° C. for 20 sec]; segment iii) 15 cycles of [98° C. for 10 sec, 60° C. for 30 sec and 72° C. for 20 sec]. The PCR product was cleaned of extra primers and dNTPs with a 15 minute incubation at 37° C. with ExoSAP-IT (GE healthcare) and then bi-directionally sequenced with primers 5′-GGCTCAGGGTCAATCACAGA-3′ (SEQ ID NO:22) and 5′-GGAAGGAGCAGTTGCGCTGC-3′ (SEQ ID NO:23) to determine the identity of the nucleotide at the predicted SNP locus.

IL28B genotypes were balanced in both arms, as shown in Tables 2-4, with the exception of the C/C group of non-responders in Arm 2 (SOC alone).

TABLE 2

| All Subjects | C/C | C/T | T/T | Total |
|---|---|---|---|---|
| Arm 1—Triple therapy | 31% | 57% | 12% | 68 |
| Arm 2—SOC alone | 27% | 57% | 16% | 63 |

TABLE 3

| Interferon-Naïve Subjects | C/C | C/T | T/T | Total |
|---|---|---|---|---|
| Arm 1—Triple therapy | 38% | 52% | 10% | 50 |
| Arm 2—SOC alone | 37% | 52% | 11% | 46 |

TABLE 4

| Non-Responders | C/C | C/T | T/T | Total |
|---|---|---|---|---|
| Arm 1—Triple therapy | 11% | 72% | 17% | 18 |
| Arm 2—SOC alone | 0% | 71% | 29% | 17 |

The results of the IL28B genotyping in interferon-naïve patients are shown in Tables 5 and 6:

TABLE 5
Influence of IL28B Alleles on ETR48 and SVR24
in Interferon-Naïve Patients

| IL28B genotype | Endpoint | Triple | SOC | Δ |
|---|---|---|---|---|
| All | ETR | 74% | 59% | 15% |
| {nₜ = 50, n_{soc} = 46} | SVR24 | 58% | 48% | 10% |
| C/C | ETR | 84% | 76% | 8% |
| {nₜ = 19 (38%), n_{soc} = 17 (37%)} | SVR24 | 74% | 65% | 9% |
| C/T | ETR | 69% | 54% | 15% |
| {nₜ = 26 (52%), n_{soc} = 24 (52%)} | SVR24 | 46% | 46% | 0% |
| T/T | ETR | 60% | 20% | 40% |
| {nₜ = 5 (10%), n_{soc} = 5 (11%)} | SVR24 | 60% | 0% | 60% |

TABLE 6
Influence of IL28B Alleles on Kinetics of HCV Clearance and SVR24 (Naives)

| | SVR when first RNA | | SVR when first RNA | | SVR when first RNA | |
|---|---|---|---|---|---|---|
| | negative | | negative D30-D85 | | negative D86-D337 | |
| IL28B | D1-D29 (RVR) | | (cEVR) | | (Slow Responder) | |
| genotype | Triple | SOC | Triple | SOC | Triple | SOC |
| CC | 10/10 (100%) | 5/6 (83%) | 4/7 (57%) | 6/10 (60%) | 0/0 | 0/0 |
| CT | 2/3 (67%) | 1/2 (50%) | 7/7 (100%) | 8/11 (73%) | 3/10 (30%) | 2/5 (40%) |
| TT | 0/0 | 0/1 | 1/2 (50%) | 0/1 | 2/3 (67%) | 0/0 |
| All | 12/13 (92%) | 6/9 (67%) | 12/16 (75%) | 14/22 (64%) | 5/13 (38%) | 2/5 (40%) |

FIG. 6 is a bar graph illustrating SVR rates according to IL28B genotype (C/C versus C/T versus T/T as compared to Overall) in interferon-naïve subjects receiving triple therapy versus SOC alone. FIG. 7 is a bar graph comparing the virologic responses (ETR and SVR) according to IL28B genotype (C/C versus C/T versus T/T as compared to Total (overall)) in interferon-naïve subjects receiving triple therapy versus SOC alone. These results show that GI-5005 triple therapy subjects with the IL28B T/T genotype had the greatest advantage in SVR.

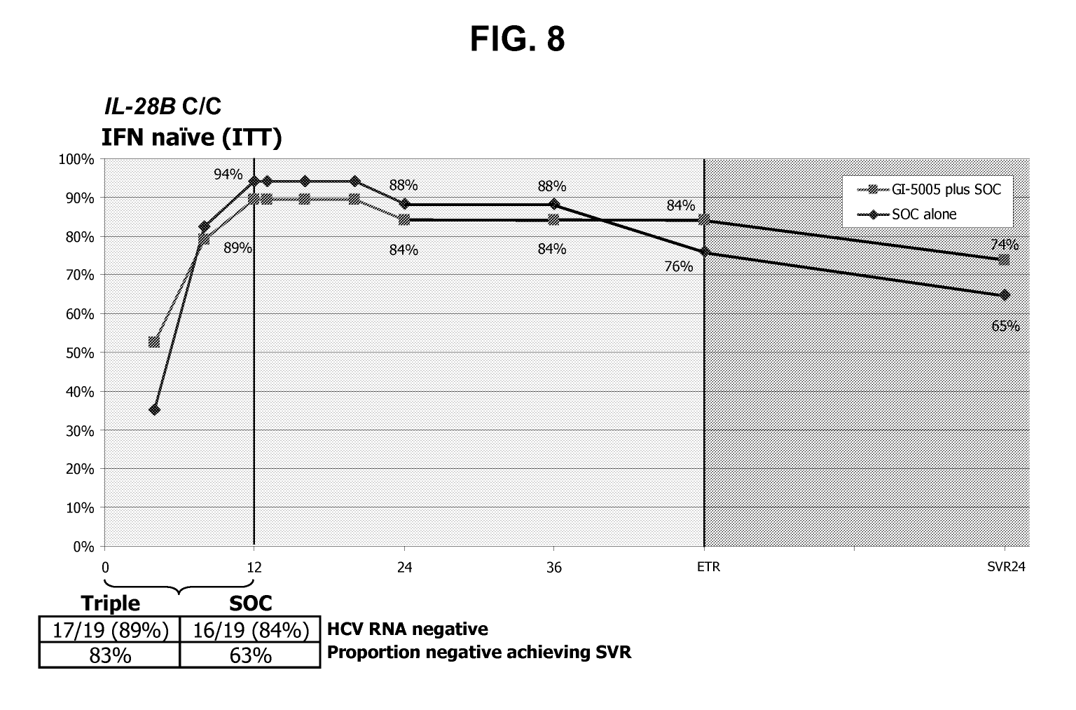

FIG. 8 is a graph showing response kinetics for interferon-naïve subjects who have the IL28B C/C genotype (triple therapy versus SOC alone), demonstrating that more IL28B C/C subjects receiving triple therapy achieved SVR than subjects receiving SOC alone (74% vs. 65%). FIG. 8 also shows that more IL28B C/C subjects receiving triple therapy and achieving viral negativity during the first 12 weeks of treatment (RVR) went on to achieve SVR than subjects receiving SOC alone and achieving viral negativity during the first 12 weeks of treatment (83% vs. 63%).

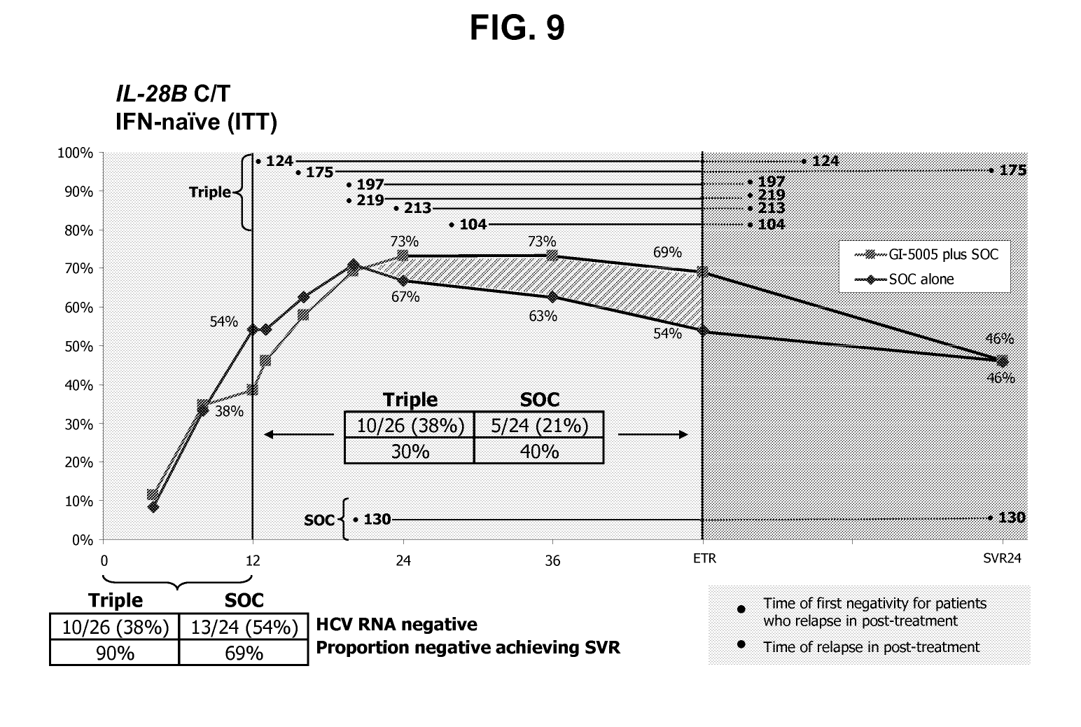

FIG. 9 is a graph showing response kinetics for interferon-naïve subjects who have the IL28B C/T genotype (triple therapy versus SOC alone), demonstrating that more IL28B C/T subjects receiving triple therapy and achieving viral negativity during the first 12 weeks of treatment (RVR) went on to achieve SVR than subjects receiving SOC alone and achieving viral negativity during the first 12 weeks of treatment (90% vs. 69%). FIG. 9 also shows that more IL28B C/T subjects receiving triple therapy reach viral negativity at ETR than IL28B C/T subject receiving SOC alone (69% vs. 54%), and that subjects who reach first viral negativity later during treatment appear to be more likely to relapse post-treatment.

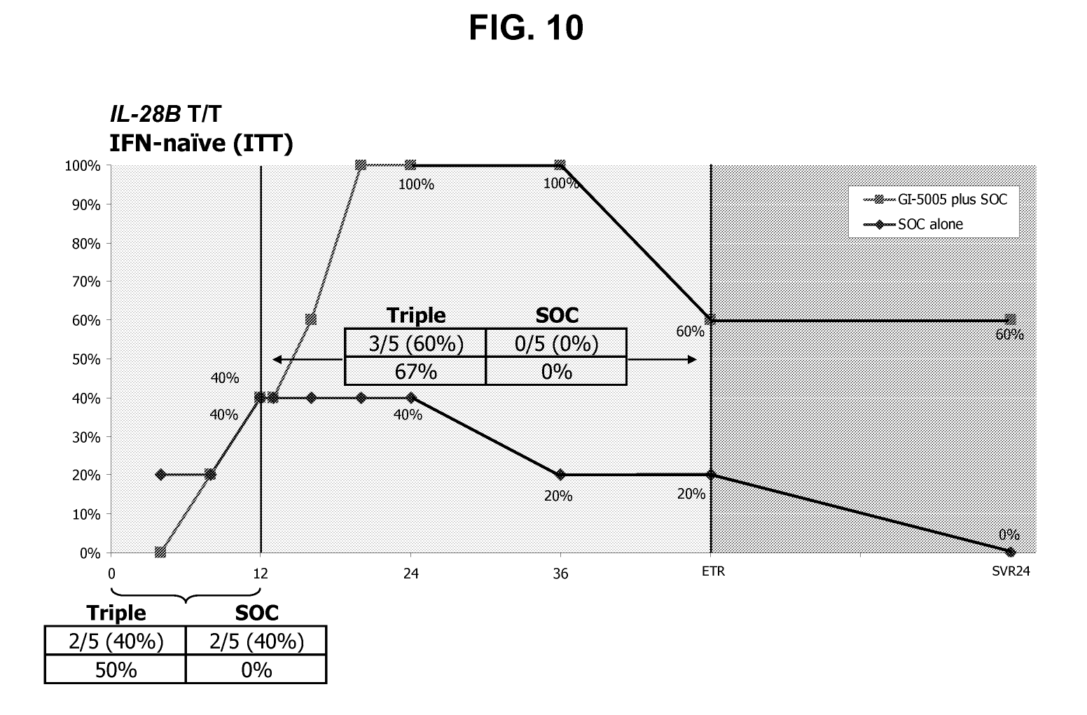

FIG. 10 is a graph showing response kinetics for interferon-naïve subjects who have the IL28B C/T genotype (triple therapy versus SOC alone), demonstrating that a significant percentage of IL28B T/T subjects receiving triple therapy achieved SVR where as no IL28B T/T subjects receiving SOC alone achieved SVR (60% vs. 0%). FIG. 10 also shows that while equal numbers of triple therapy and SOC alone IL28B T/T subjects achieved viral negativity during the first 12 weeks of treatment (RVR), only those receiving triple therapy went on to achieve SVR (50% vs. 0%). FIG. 10 also shows that IL28B T/T subjects receiving triple therapy continued to achieve viral negativity after the first 12 weeks of treatment, whereas no additional IL28B T/T subjects receiving SOC alone achieved viral negativity after the first 12 weeks of treatment.

The results of the IL28B genotyping in non-responder patients are shown in Table 7:

TABLE 7
Influence of IL28B Alleles on ETR
and SVR24 in Non-Responder Patients

| IL28B genotype | Endpoint | Triple | SOC | Δ |
|---|---|---|---|---|
| All | ETR | 33% | 11% | 22% |
| {nₜ = 18, n_{soc} = 19} | SVR24 | 17% | 5% | 12% |
| C/C | SVR24 | 0% | 0% | 0% |
| {nₜ = 2, n_{soc} = 0} | | | | |
| C/T | SVR24 | 23% | 8% | 15% |
| {nₜ = 13, n_{soc} = 13} | | | | |
| T/T | SVR24 | 0% | 0% | 0% |
| {nₜ = 3, n_{soc} = 5} | | | | |

The results of the IL28B genotyping in all patients are shown in Table 8:

TABLE 8
Influence of IL28B Alleles on SVR24 in All Patients

| IL28B genotype | Endpoint | Triple | SOC | Δ |
|---|---|---|---|---|
| All | ETR | 63% | 45% | 18% |
| {nₜ = 68, n_{soc} = 65} | SVR24 | 47% | 35% | 12% |
| C/C | SVR24 | 67% | 65% | 2% |
| {nₜ = 21, n_{soc} = 17} | | | | |
| C/T | SVR24 | 38% | 32% | 6% |
| {nₜ = 39, n_{soc} = 37} | | | | |
| T/T | SVR24 | 38% | 0% | 38% |
| {nₜ = 8, n_{soc} = 10} | | | | |
| IL28B Status Unknown | SVR24 | 0% | 0% | 0% |
| {nₜ = 0, n_{soc} = 1} | | | | |

The results of these studies demonstrate that pharmacogenomic analyses can provide valuable insights into therapeutic trial results. In interferon-naïve patients, triple therapy improved ETR regardless of IL28B genotype; delivering more C/C and C/T RVRs, more C/T and T/T cEVRs, and more T/T slow responders. The effect of GI-5005 on SVR is greatest in patients with the poorest prognosis genotype (TT).

In prior non-responders, triple therapy improved outcomes in this patient group as a whole, which was a result of SVRs attained in IL28B C/T genotype patients. It is noted that the non-responder group in this study consisted only of prior partial responders to interferon therapy (defined in this study as patients achieving >2 log reduction in virus by at least 12 weeks but no clearance after 24 weeks) and prior poor responders (defined in this study as patients achieving between 1 and 2 log reduction in virus by at least 12 weeks of therapy but no clearance after 24 weeks), but excluded null responders, relapsers, or treatment-breakthrough patients, which are sometimes included in “non-responder” groups in other studies. Since prior relapsers and prior treatment-breakthrough patients (see General Definitions) can generally be expected to perform better in response to re-treatment as compared to other types of non-responders, the non-responders in the present study are believed to represent a subpopulation of chronically infected patients that is particularly difficult to treat.

Taken together, the IL28B genotyping indicates that the GI-5005 therapeutic vaccine augments therapeutic response in those with unfavorable IL28B types, supporting the combination of immunotherapy with SOC for the treatment of infectious disease such as HCV infection, as well as the use of immunotherapy with other HCV inhibitory agents for the treatment of such diseases.

C/Cs, which are individuals having the C/C genotype at the IL28B locus (described in more detail below), are predicted to have the best prognosis for responding to SOC therapy (approximately 78% of these individuals will achieve SVR in response to SOC) (Ge et al., supra). In addition, C/C individuals are the most likely to spontaneously clear an HCV infection (Thomas et al., supra). The inventors have now discovered how C/C individuals respond to immunotherapy. A substantial number of C/C individuals responded to triple therapy early in treatment (the first 12 weeks), and the same was true for C/C individuals receiving SOC; however, a greater percentage of C/C individuals receiving triple therapy who reached RVR or cEVR went on to achieve complete responses at the end of treatment and SVR, as compared to C/C individuals receiving SOC alone. Therefore, while C/C individuals generally respond well to both triple therapy and SOC alone and with similar overall kinetics, triple therapy delivered substantially more C/C patients to complete response by the ETR and SVR endpoints (see FIG. 8).

C/Ts, who are individuals having the heterozygous C/T genotype at the IL28B locus (described in detail below), are predicted to have a moderate prognosis of responding to SOC therapy (approximately 37% of these individuals will achieve SVR in response to SOC therapy) (Ge et al., supra. The present invention provides evidence that the response rates of C/T individuals can be substantially improved by using immunotherapy. More particularly, although both triple therapy and SOC C/T interferon-naïve individuals achieved the same rates of SVR in the study described herein (see FIG. 9), examination of the response kinetics (see FIG. 9) reveals characteristics of the response kinetics to immunotherapy (triple therapy) that can now be used to improve the response of C/Ts. Specifically, in both triple therapy and SOC alone, C/Ts had a later time course to complete response, showing increased numbers of individuals reaching viral negativity after the first 12 weeks. However, the SOC treatment group lost C/T responders on therapy (i.e., between weeks 24-48), whereas the triple therapy treatment group substantially maintained complete responses in C/Ts during this same period of time on therapy, achieving a better ETR for C/Ts on triple therapy (see FIG. 9). It was only after treatment ended at 48 weeks (ETR) that C/Ts in the triple therapy group experienced enough relapses to move the total percentage of complete responses at SVR to the same rate as C/Ts on SOC alone (notably, C/Ts in the SOC group also lost responders post-treatment). A review of C/T individuals who first achieved viral negativity during the 24-48 week period and who subsequently relapsed post-treatment provides additional insight into the response of these individuals to immunotherapy. Specifically, referring to FIG. 9, in the triple therapy group, the later that an individual achieves viral negativity on treatment, the sooner the individual appears to relapse post-treatment. These data indicate that C/Ts respond more slowly to therapy (either type) and while on triple therapy, appear to maintain viral negativity, in contrast to SOC. Moreover, among prior non-responders to interferon-based therapy, triple therapy delivered 23% of patients to SVR, as compared to only 8% of the patients receiving SOC alone.

Based on the results provided herein, the present inventors believe that C/Ts receiving immunotherapy, particularly those who are slow responders to triple therapy (e.g., those who respond after 12 weeks of triple therapy), should continue receiving triple therapy beyond the standard end of treatment at 48 weeks. By extending and/or modifying triple therapy, it is believed that a substantially larger percentage of C/T individuals will achieve complete response at SVR. Moreover, the data presented herein shows that single patients can be monitored for responsiveness under immunotherapy-based regimens and their therapy can be personalized by extending or modifying treatment based on genotype combined with first time to responsiveness, in order to optimize their chance of achieving a complete response to the therapy. Prior to the present invention, such a personalized approach, or response-guided approach, to therapy for HCV and other infectious disease was not available.

The effect of immunotherapy on outcome of therapy was the greatest in patients with the poorest prognosis genotype (T/T). T/Ts, who are individuals having the T/T genotype at the IL28B locus (described in detail below), are predicted to have a poor prognosis of responding to SOC therapy (only approximately 26% of these individuals will achieve SVR in response to SOC therapy) (Ge et al., supra). The present invention provides evidence that the response rates of T/T individuals can be significantly improved by using immunotherapy. More particularly, both ETR and SVR rates in T/T patients were significantly greater for triple therapy compared to SOC or historical controls (see FIG. 10), with 60% of the T/T patients achieving ETR and maintaining negativity to SVR, demonstrating that immunotherapy has a substantial impact in this high risk patient group. Triple therapy delivered patients who reached viral negativity prior to 12 weeks or after 12 weeks (slow responders) to SVR, whereas SOC alone did not in this study. All T/T patients in the triple therapy group reached viral negativity by 24 weeks. Compared to the dismal SVR rate of 26% reported historically for SOC alone and of 0% reported in this study, immunotherapy demonstrated that the outcomes of this subgroup of patients can be changed from poor to good. Accordingly, by simply adding immunotherapy to standard regimens such as SOC or new regimens that may include other antivirals and interferons, T/T patients can be treated with a greater likelihood of having a positive outcome. In addition, the results described herein indicate that in this subgroup of patients, as with the C/T genotype patients described above, some of the T/T patients achieved negatively later in therapy, after the 12 week EVR endpoint that is used in SOC as a predictor for positive outcomes. Such patients, if treated for an extended period of time (e.g., longer than 48 weeks total), can be expected to have an improved likelihood of reaching SVR as compared to patients for whom the standard SOC protocol is utilized. Accordingly, T/T patients can be monitored for responsiveness under immunotherapy-based regimens, and their therapy can be personalized by extending and/or modifying treatment based on genotype combined with first time to responsiveness, in order to optimize their chance of achieving a complete response to the therapy.

### Example 3

The following example demonstrates that immunotherapy in combination with SOC improves liver function in individuals chronically infected with hepatitis C virus.

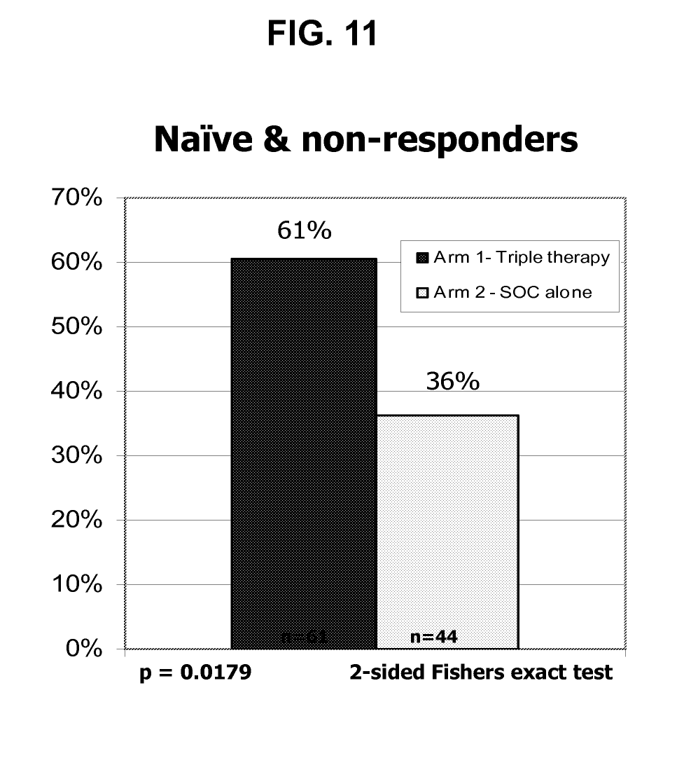

“ALT” is a well-validated measure of hepatic injury and serves as a surrogate for hepatic inflammation. In prior large hepatitis trials, reductions and/or normalization of ALT levels (ALT normalization) have been shown to correlate with improved liver function and reduced liver fibrosis as determined by serial biopsy. Patients in the phase 2 clinical trial for chronic HCV infection were examined for ALT levels. ALT normalization results at end of treatment (all patients) and SVR24 (interferon-naïve subjects) is shown in FIGS. 11-13.

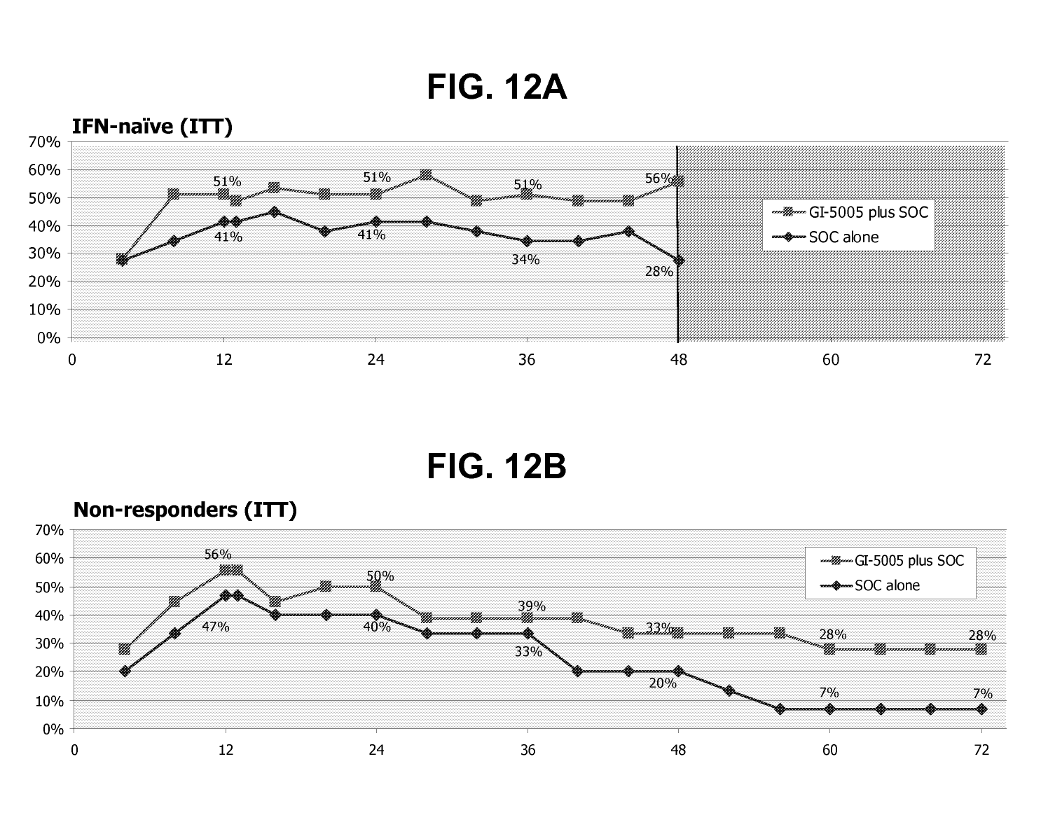

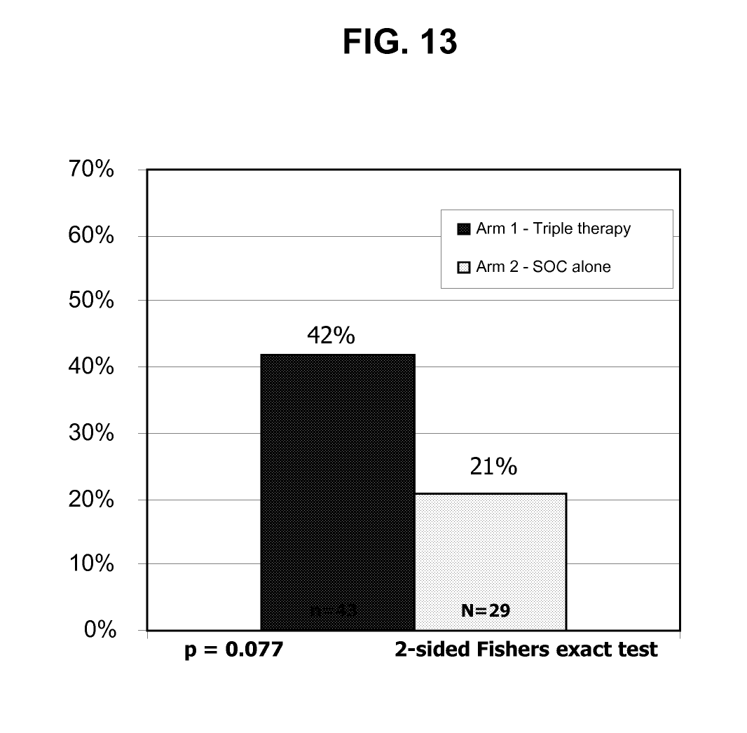

FIG. 11 is a bar graph showing that at end of treatment, the group of interferon-naïve and non-responders on triple therapy had improved ALT normalization as compared to subjects receiving SOC alone (61% vs. 36%). FIG. 12A shows that at end of treatment for interferon-naïve (IFN-naïve) subjects (48 weeks), triple therapy demonstrated an improvement in ALT normalization as compared to subjects receiving SOC alone (56% vs. 28%). FIG. 12B shows that at end of treatment for Non-responder subjects (72 weeks), triple therapy demonstrated an improvement in ALT normalization as compared to subjects receiving SOC alone (28% vs. 7%). FIG. 13 is a graph showing that at 24 weeks post-treatment (SVR24), interferon-naïve subjects who received triple therapy demonstrated a sustained improvement in ALT normalization as compared to subjects receiving SOC alone (42% vs. 21%).

### Example 4

The following example describes the impact of adding immunotherapy to SOC on HCV-specific T cell responses in subjects with the IL28B T/T genotype.

Peripheral blood mononuclear cells (PBMCs) were obtained from the patients in the clinical study described in Examples 1-3 above. HCV-specific T-cell activation was analyzed by interferon-γ ELISpot assay of peripheral blood mononuclear cells (PBMCs) stimulated ex vivo with HCV peptide antigens. The magnitude, breadth and specificity of the T cell response was evaluated using the panel of peptides.

More specifically, for ELISpot immune analysis, peripheral blood mononuclear cells (PBMCs) isolated from patients in the study were cryopreserved until assay. All timepoints from a subject were assayed on the same day (longitudinal analysis). At the time of assay, the PBMCs were thawed and incubated with HCV peptides ex vivo. The peptides comprised a panel of 407 overlapping peptides (15 to 20 mers) spanning all expressed HCV proteins. A second panel of 93 peptides (8 to 12 mers) identified as cognate peptides for human CD8+ T cell responses was also analyzed.

T cell responses were analyzed by IFNγ production (hallmark of T cell activation). Phorbol myristate acetate (PMA) plus ionomycin was added as a positive control. Medium alone was added to six replicate wells to generate control background values, and cells (or “spots”) per million PBMCs that produce IFNγ were enumerated. The cores were adjusted by subtracting the average background value from each peptide pool and also by correcting the peptide pool score at each timepoint by subtraction of the baseline value for that given peptide pool.

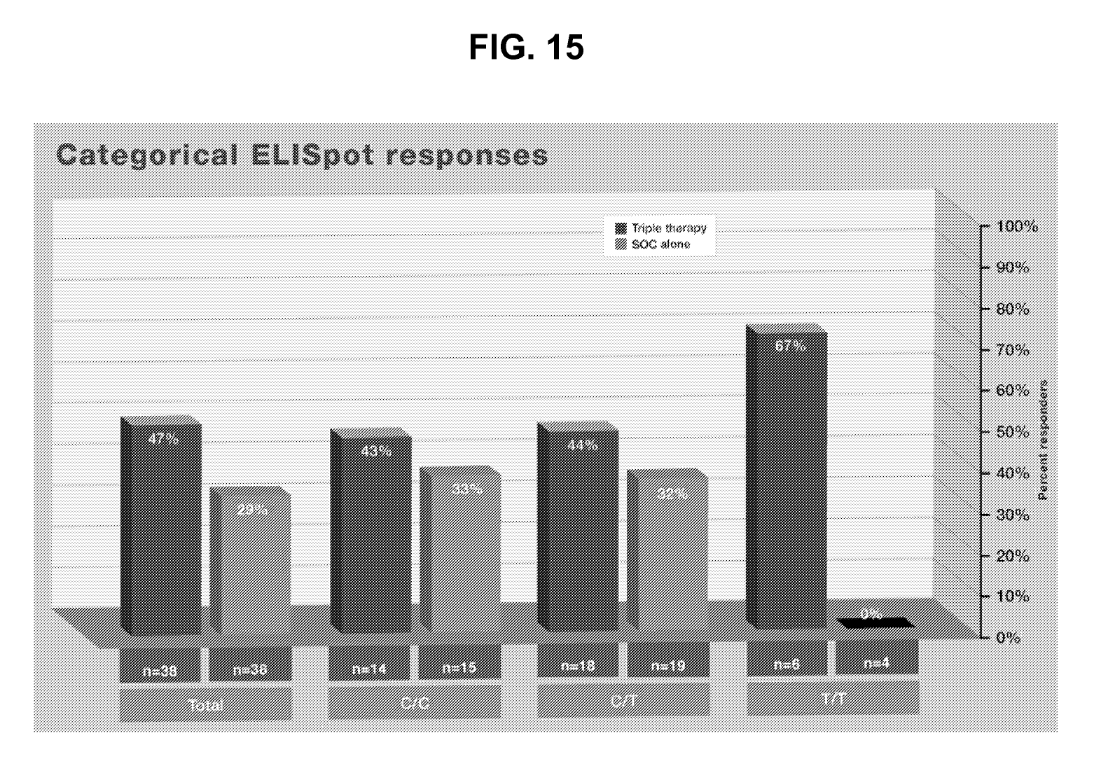

The categorical cellular immune responses for interferon-naïve subjects, represented by IL28B subgroup and overall (total) are shown in FIG. 15. A “categorical immune response” is an algorithm that was pre-specified to evaluate the IFNγ T cell response in terms of breadth, duration and magnitude, since it has been previously shown that acutely infected HCV subjects generate T cell responses that are robust in magnitude and show broad HCV epitope recognition (Rehermann and Chisari, Current Topics in Microbiology and Immunology (2000) 242: 299-325; Lauer et al., Gastroenterology (2004) 127: 924-936; Lechner et al., Journal of Experimental Medicine (2000) 191: 1499-1512).

For a subject to be deemed a responder in the ELISpot assay, the following stringent criteria had to be met:

1) Overlapping (non-optimized) peptide ELISpot:
15 or more pools >25 spots at one visit;
Or at least 10 pools >25 spots at one visit with at least 2 pools positive (>25) on more than one on-treatment measurement;
Or at least 5 pools >25 spots at one visit with at least one pool >150 spots.

2) Non-overlapping, discrete (optimized) peptide ELISpot:
4 or more pools >75 spots at one visit;
Or at least 2 pools >75 spots with at least 1 pool positive (>75) for more than one on-treatment measurement;
Or at least 2 pools >75 spots with at least 1 pool >150 spots.

The IFNγ responses were evaluated by different treatment periods through the monotherapy run-in (GI-5005 treated subjects only), triple therapy or SOC, and the post treatment follow-up period.

For these ELISpot assays, a total of 76 subjects were analyzed with representation of the IL28B genotypes as follows:

| | C/C | C/T | T/T |
|---|---|---|---|
| Triple Therapy | 14 | 18 | 6 |
| SOC | 15 | 19 | 4 |

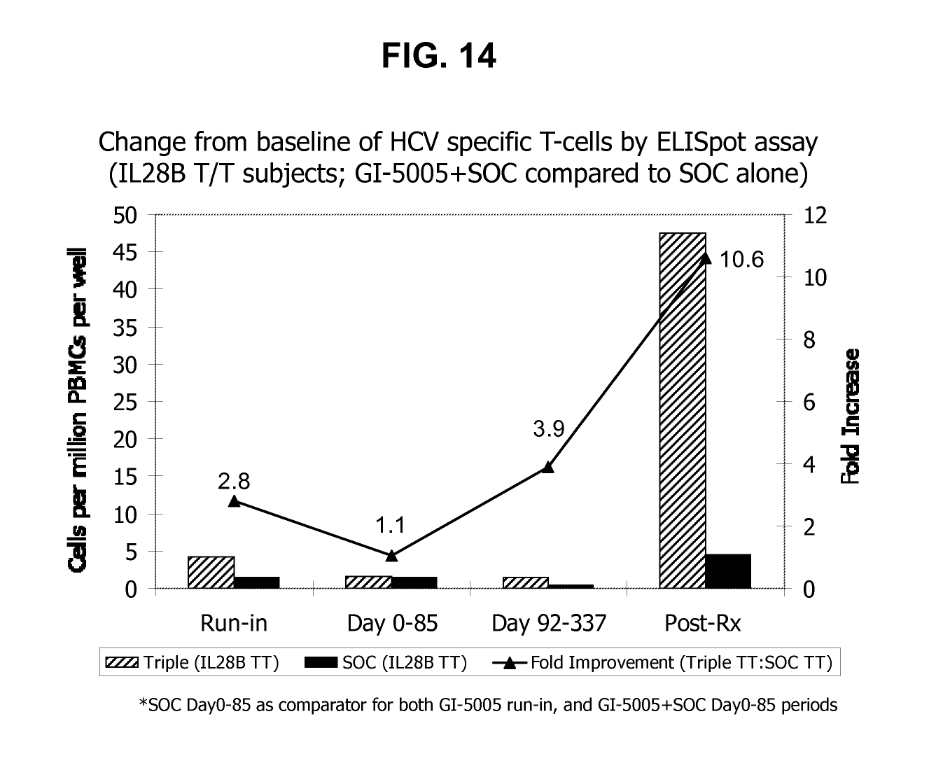

As discussed above, analysis of ELISpot results used pre-specified parameters established to evaluate the IFNγ T cell response in terms of breadth, duration and magnitude. The results demonstrated that in subjects receiving SOC alone, HCV-specific cellular immune responses were as much as 17-fold lower in IL28B T/T subjects compared to other IL28B subgroups (0.4 vs 6.6 T-cells/106 PBMCs/well). In subjects receiving triple therapy (GI-5005+SOC), HCV-specific cellular immune responses were up to 5-fold higher in IL28B T/T subjects as compared to other IL28B subgroups (47.5 vs 8.9 T-cells/106 PBMCs/well). Moreover, HCV-specific T-cell responses were increased by up to 10-fold in IL28B T/T subjects receiving triple therapy compared to IL28B T/T subjects receiving SOC alone (47.5 vs 4.5 T-cells/106 PBMCs/well; see FIG. 14). Improved HCV-specific immunity in triple therapy IL28B T/T subjects correlated with the improvement in SVR (60% vs. 0%) compared to IL28B T/T subjects treated with SOC alone, described in Example 2 above.

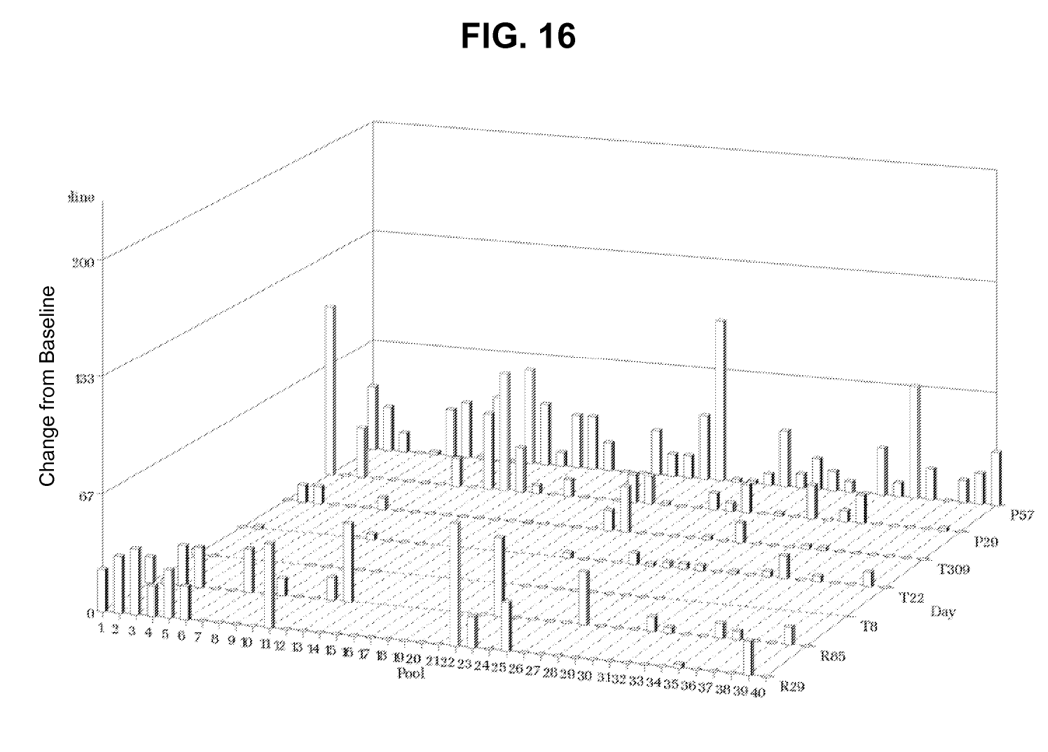

Using the categorical analysis, GI-5005 combined with SOC generated enhanced immune responses in IL28B T/T naïve subjects compared to SOC (see FIG. 15). Four of 6 T/T subjects (67%) demonstrated T cell responses in the Triple therapy arm compared to none (0 out of 5) in SOC (see FIG. 15). In addition, cellular immune responses were demonstrated at 18% increased absolute frequency in subjects across all IL28B genotypes receiving Triple therapy compared to subjects receiving SOC (47% vs. 29%). FIG. 16 shows a representative example of the response to the overlapping (non-optimized) peptide pools for one IL-28B T/T subject in the triple therapy arm. The increase from baseline at multiple timepoints in IFN-γ producing cells per million PBMCs (y-axis) is plotted against the peptide pools that were used for stimulation (x-axis). As shown in this figure, over time, the immune response increases both in magnitude and with respect to the HCV peptides against which a cellular immune response is elicited.

In summary, important differences were noted for the different IL28B genotypes related to the timing and magnitude of HCV specific cellular immunity as measured by IFNγ ELISpot assay. GI-5005 triple therapy improved HCV specific cellular immunity as measured by IFNγ ELISpot assay in all IL28B subgroups (C/C; 43% vs 33%, C/T; 44% vs 32%, T/T; 67% vs 0%) as well as end of treatment viral clearance (C/C; 84% vs 76%, C/T; 69% vs 54%, T/T; 60% vs 20%) and SVR in C/C (74% vs 65%) and T/T groups (60% vs 0%). The greatest favorable treatment effect for GI-5005 was observed in the T/T group (ETR +40% and SVR +60%). The low levels of HCV specific cellular immune responses measured in the SOC IL28B T/T group suggest that poor cellular immunity may be the most significant deficit in these patients, and point to new models of pathogenesis and response to antiviral therapy as described herein.

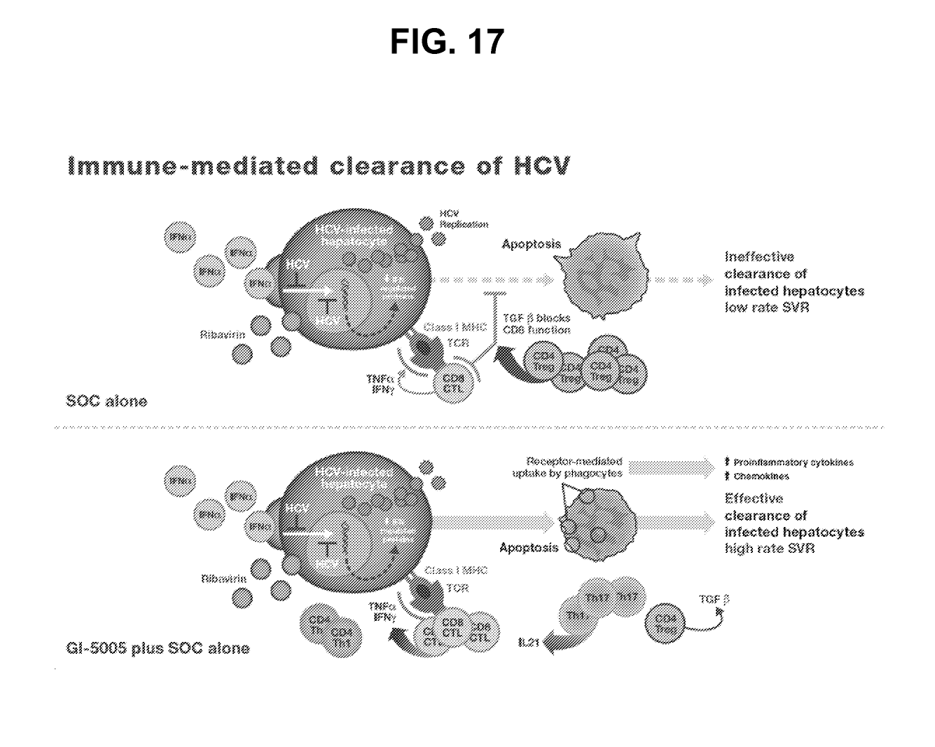

FIG. 17 shows a hypothetical model of yeast-based immunotherapy effects on immune-mediated hepatic clearance. Without being bound by theory, it is proposed that when SOC alone is administered to subjects chronically infected with HCV, SOC inhibits viral replication, but hepatic clearance is poor due to low numbers of HCV-specific CD4+ and CD8+ T cells in the liver and suppression of these effector T cells by suppressive regulatory T cells (Tregs). In contrast, when triple therapy (immunotherapy and SOC) is administered to chronically infected subjects, the number and functionality of effector CD4+ and CD8+ T cells in the liver are increased/activated. In addition, yeast-based immunotherapy reduces the number and functionality of Tregs via induction of the Th17 pathway, further unbridling the favorable CD4/CD8 effects. Th17 cells also produce IL21, which increases the longevity of CD8+ T cells, all of which contributes to an effective hepatic clearance and higher SVR rates.

These examples demonstrate that triple therapy subjects with IL28B T/T genotype had the greatest advantage in SVR as well as IFN-γ ELISpot assay, indicating that the immunotherapeutic element, GI-5005, is compensating for a deficit in T cell immunity in these subjects. SOC IL28B T/T subjects had notably poorer virologic and IFN-γ ELISpot responses than SOC C/C and C/T patients, indicating that the fundamental deficit in these patients is one of cellular immune response. The relationship of GI-5005 immune and virologic response to IL28B status suggests an alternate model of pathogenesis for chronic HCV where differences in HCV-specific cellular immunity play a major role in driving sustained response to antiviral therapy. The consistency of the clinical immunology and virologic data provides further confidence in the SVR advantage observed in this phase 2 clinical trial.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

1. A method to treat chronic hepatitis C virus (HCV) infection, and/or to prevent, ameliorate or treat at least one symptom of chronic HCV infection, in an individual having an IL28B genotype of C/T or T/T, the method comprising administering to the individual a yeast-based immunotherapeutic composition comprising at least one HCV antigen or immunogenic domain thereof and one or both of at least one interferon and at least one anti-viral compound, wherein the immunotherapeutic composition and the interferon and/or anti-viral compound are administered concurrently for a period of time that is longer than the period of time established as effective for the interferon and/or anti-viral compound in the absence of the yeast-based immunotherapy.

2. A method to treat chronic hepatitis C virus (HCV) infection, and/or to prevent, ameliorate or treat at least one symptom of chronic HCV infection, in an individual having an IL28B genotype of C/T or T/T, the method comprising administering to the individual a therapeutic protocol comprising yeast-based immunotherapeutic composition comprising at least one HCV antigen or immunogenic domain thereof and one or both of at least one interferon and at least one anti-viral compound, wherein the virus level is monitored in the individual, and, when the individual first achieves viral negativity, the individual is treated for an additional 4 to 48 weeks with the therapeutic protocol.

3. (canceled)

4. The method of claim 1, wherein the immunotherapeutic composition and the interferon and/or anti-viral compound are administered concurrently for at least several weeks longer than the period of time established as effective for the interferon and/or anti-viral compound in the absence of the yeast-based immunotherapy.

5. The method of claim 1, wherein the immunotherapeutic composition and the interferon and/or anti-viral compound are administered concurrently for at least 4 to 48 weeks longer than the period of time established as effective for the interferon and/or anti-viral compound in the absence of the yeast-based immunotherapy.

6. The method of claim 1, wherein the interferon is pegylated interferon-α.

7. (canceled)

8. The method of claim 1, wherein the anti-viral compound is ribavirin.

9. The method of claim 1, wherein the anti-viral compound includes ribavirin and an HCV protease inhibitor.

10. (canceled)

11. (canceled)

12. A method to treat chronic hepatitis C virus (HCV) infection, and/or to prevent, ameliorate or treat at least one symptom of chronic HCV infection, in an individual comprising administering to an individual:
a) a yeast-based immunotherapeutic composition comprising at least one HCV antigen or immunogenic domain thereof, wherein the immunotherapeutic composition elicits a T cell-mediated immune response against one or more HCV antigens;
b) pegylated interferon-α; and
c) ribavirin;
wherein the immunotherapeutic composition, the pegylated interferon-α, and the ribavirin are administered concurrently over a period of 48 weeks to interferon-naïve individuals having an IL28B genotype of C/C, and over a period of 72 weeks to non-responder individuals having an IL28B genotype of C/C, except that the agents of (b) and/or (c) may optionally be administered in reduced dose, reduced frequency, or for a shorter period of time than the protocol established as effective for the agents of (b) and/or (c), respectively, in the absence of immunotherapy;
wherein the immunotherapeutic composition, the pegylated interferon-α, and the ribavirin are administered concurrently over a period of 48 weeks to interferon-naïve individuals having an IL28B genotype of C/T or T/T, and over a period of 72 weeks to non-responder individuals having an IL28B genotype of C/T or T/T, except that, if the individual having an IL28B genotype of C/T or T/T does not reach viral negativity within the first 12 weeks of the period, then the immunotherapeutic composition, the pegylated interferon-α, and the ribavirin are administered for a period greater than 48 weeks for interferon-naïve individuals and for a period greater than 72 weeks for non-responder individuals.

13. (canceled)

14. (canceled)

15. The method of claim 1, wherein the fusion protein comprises SEQ ID NO:2.

16. A method to treat hepatitis virus infection in an individual, comprising treating the individual with a therapeutic protocol comprising administration of:
a) a yeast-based immunotherapeutic composition comprising at least one hepatitis virus antigen or immunogenic domain thereof, wherein the immunotherapeutic composition elicits a T cell-mediated immune response against one or more hepatitis virus antigens; and
b) one or more agent selected from: an interferon, an anti-viral compound, a host enzyme inhibitor, and/or an immunotherapeutic composition other than the yeast-based immunotherapeutic composition of (a);
wherein the therapeutic protocol is modified for individuals having an IL28B genotype of C/C by reducing the dose and/or frequency and/or period of time of administration of one or more of the agents of (b) as compared to the dose and/or frequency and/or period of time of administration established as effective for the agents of (b) in the absence of yeast-based immunotherapy;
wherein the therapeutic protocol is modified for individuals having an IL28B genotype of C/T or T/T by monitoring the responsiveness of these individuals to the protocol and extending the period of time of administration of the protocol for those individuals who are slow responders to the protocol.

17. The method of claim 16, wherein the hepatitis virus is hepatitis C virus (HCV) or hepatitis B virus (HBV).

18. (canceled)

19. A method to treat chronic hepatitis B virus (HBV) infection, and/or to prevent, ameliorate or treat at least one symptom of chronic HBV infection, in an individual comprising administering to an individual:
a) a yeast-based immunotherapeutic composition comprising at least one HBV antigen or immunogenic domain thereof, wherein the yeast-based immunotherapeutic composition elicits a T cell-mediated immune response against one or more HBV antigens;
b) one or more agents selected from interferon, lamivudine, adefovir, tenofovir, telbivudine, and entecavir;
wherein the yeast-based immunotherapeutic composition and the one or more agents are administered concurrently to individuals having an IL28B genotype of C/C until the individual reaches seroconversion, except that the agents of (b) may optionally be administered in reduced dose, reduced frequency, or for a shorter period of time than the protocol established as effective for the agents of (b) in the absence of yeast-based immunotherapy, followed optionally, by an additional period of administration of the agents of (a) and/or (b) for 1 to 12 months;
wherein the yeast-based immunotherapeutic composition and the one or more agents are administered concurrently to individuals having an IL28B genotype of C/T or T/T until the individual reaches seroconversion, followed by an additional period of administration of the agents of (a) and/or (b) for 1 to 12 months.

20. (canceled)

21. A method to treat an infectious disease in an individual comprising treating the individual with a therapeutic protocol comprising administration of a yeast-based immunotherapeutic composition, wherein the IL28B genotype of the individual is determined prior to administering the protocol;
wherein the time of administration of the therapeutic protocol is lengthened for individuals having a genotype of IL28B C/T or T/T who first respond to the therapeutic protocol later than the average time period for response for all individuals or for individuals having an IL28B genotype of C/C; and/or
wherein the therapeutic protocol is modified for individuals having an IL28B genotype of C/C by reducing the dosage, duration of administration, or the frequency of administration of one or more agents in the therapeutic protocol other than the yeast-based immunotherapeutic composition.

22. (canceled)

23. The method of claim 21, wherein the infectious disease is a viral disease.

24. The method of claim 21, wherein the infectious disease is hepatitis virus infection.

25. (canceled)

26. (canceled)

27. (canceled)

28. (canceled)

29. (canceled)

30. The method of claim 1, wherein the yeast vehicle is a whole yeast.

31. The method of claim 1, wherein the yeast vehicle is a heat-inactivated yeast.

32. The method of claim 1, wherein the yeast vehicle is from Saccharomyces cerevisiae.

33. (canceled)

34. (canceled)
